(19) United States
(12) Patent Application Publication
(10) Pub. No.: US 2025/0206824 A1
(43) Pub. Date: Jun. 26, 2025

(54) GLYCOENGINEERED Fc VARIANT POLYPEPTIDES WITH ENHANCED EFFECTOR FUNCTION

(71) Applicant: ABLYNX N.V., Zwijnaarde (BE)

(72) Inventors: Julie A. Jaworski, Cambridge, MA (US); Sagar V. Kathuria, Cambridge, MA (US); Sunghae Park, Cambridge, MA (US); Qun Zhou, Cambridge, MA (US)

(21) Appl. No.: 18/970,395

(22) Filed: Dec. 5, 2024

Related U.S. Application Data
(63) Continuation of application No. 18/493,991, filed on Oct. 25, 2023.
(60) Provisional application No. 63/419,188, filed on Oct. 25, 2022.

(51) Int. Cl. C07K 16/28; C12N 15/63

(52) U.S. Cl. CPC C07K 16/283; C12N 15/63; C07K 2317/14; C07K 2317/24; C07K 2317/31; C07K 2317/52; C07K 2317/569; C07K 2317/622; C07K 2317/732; C12N 2501/999

(57) ABSTRACT

The present disclosure provides glycoengineered Fc domain variants comprising one or more oligomannose-type N-glycans and an Fc domain mutation. The present disclosure also provides nucleic acids encoding Fc domain variants and host cells for making Fc domain variants. Methods for increasing the yield of Fc domain variants, and methods of using Fc domain variants to treat disease, are also provided.

### RELATED APPLICATIONS

This application is a continuation of U.S. patent application Ser. No. 18/493,991, filed Oct. 25, 2023, which claims priority to U.S. Provisional Patent Application Ser. No. 63/419,188, filed Oct. 25, 2022, the disclosures of which are hereby incorporated by reference in their entirety.

### BACKGROUND

Therapeutic antibodies have been used extensively in clinics for treating patients with many challenging diseases, including cancer. See Redman J M et al. (2015), Mechanisms of action of therapeutic antibodies for cancer. Mol Immunol. vol. 67 (2 Pt A): 28-45. It has been demonstrated that many of them act through effector functions, with antibody-dependent cellular cytotoxicity (ADCC) as a major mechanism. See Bournazos S et al. (2017), Signaling by Antibodies: Recent Progress. Annual Review of Immunology, vol. 35:285-311.

In ADCC, the antibody-antigen interaction results in increased affinity of IgG-Fc domains for FcγRIIIa expressed on natural killer (NK) cells, leading to signaling transduction and cellular degranulation, and subsequent killing of the target cells. Besides the cytotoxicity for pathogens and cancer cells, ADCC was recently demonstrated to be involved in the immune modulation of several checkpoint inhibitors. See Ingram J R et al. (2018),), Proc. Natl. Acad. USA, vol. 115 (15): 3912-7 and Goletz C et al. (2018), Frontiers in immunology, vol. 9:1614. ADCC is also required for high efficacy of antibodies against certain autoimmune diseases. See Bloemendaal F M et al. (2017), Gastroenterology, vol. 153 (5): 1351-62.e4.

Many efforts have been made to enhance ADCC for increasing the efficacy in the treatment against diseases. For example, protein engineering of the Fc-CH2 domain using site-directed mutagenesis can significantly increase FcγRIIIa binding and ADCC activity. See Lazar G A et al. (2006), Proc. Natl. Acad. USA, vol. 103 (11): 4005-10. Further modifications of the Fc domain may lead to further enhancements in FcγRIIIa binding and ADCC activity. Nevertheless, despite these efforts, there remains a need to create new Fc variant antibodies and other binding polypeptides with enhanced efficacy and improved manufacturability for use in treating various diseases.

### SUMMARY

The present disclosure is directed in part to the discovery that certain glycoengineered Fc domain variants (e.g., oligomannose-modified Fc variants) have improved manufacturability and effector function as compared to conventional Fc variant domains. Accordingly, the present disclosure is further directed in part to the glycoengineering (e.g., using kifunensine and related glycosylation inhibitors) of Fc variant polypeptides to improve their manufacturability and utility as therapeutic agents.

In one aspect, a composition comprising a population of isolated glycosylated binding polypeptides each comprising an Fc domain comprising an N-glycan, wherein the Fc domain further comprises at least one of the following mutations: (i) to (ix) according to EU numbering:
(i) an aspartic acid (D) at amino acid position 239,
(ii) an aspartic acid (D) at amino acid position 267,
(iii) an aspartic acid (D) or glutamic acid (E) at amino acid position 268,
(iv) an alanine (A) or a cysteine (C) at amino acid position 298,
(v) an isoleucine (I), a methionine (M), a glutamine (Q), or a tryptophan (W) at amino acid position 314,
(vi) a phenylalanine (F) or a methionine (M) at amino acid position 330,
(vii) a glutamic acid (E) at amino acid position 332,
(viii) an aspartic acid (D), an isoleucine (I), a proline (P), or a threonine (T) at amino acid position 339, or
(ix) a phenylalanine (F) or a tryptophan (W) at amino acid position 373, and wherein the composition comprises at least 50% Man₅₋₉(GlcNAc)₂ N-glycans by molar ratio, relative to all N-glycans, is provided.

In certain exemplary embodiments, Man₈ and Man₉ together are the major species of Man₅₋₉ (GlcNAc)₂ N-glycans in the composition.

In some exemplary embodiments, the composition comprises greater than 70%, 75%, 80%, 85%, 90%, or 95% Man₉(GlcNAc)₂ N-glycans by molar ratio, relative to all N-glycans.

In still other exemplary embodiments, the composition comprises at least 97% Man₉(GlcNAc)₂ N-glycans by molar ratio, relative to all N-glycans.

In certain exemplary embodiments, at least 80% of the N-glycans by molar ratio, relative to all N-glycans in the composition are afucosylated.

In other exemplary embodiments, the binding polypeptides of the composition are produced by culturing cells that express the binding polypeptides in the presence of a mannosidase inhibitor. In some embodiments, the mannosidase inhibitor is kifunensine. In certain embodiments, the concentration of kifunensine is from about 60 ng/ml to about 2500 ng/ml. In one exemplary embodiment, the concentration of kifunensine is about 2000 ng/ml.

In certain exemplary embodiments, the binding polypeptides of the composition comprising Man₅₋₉(GlcNAc)₂ N-glycans have increased affinity for binding to an Fcγ receptor compared to a reference polypeptide that does not comprise Man₅₋₉ (GlcNAc)₂ N-glycans but is otherwise identical. In some exemplary embodiments, the Fcγ receptor is human FcγRIIIa. In particular exemplary embodiments, the binding polypeptides of the composition comprising Man₅₋₉ (GlcNAc)₂ N-glycans have increased affinity for binding to human FcγRIIIa of at least 2, 3, 4, 5, 10, 20, 30, 40, 50, 60, 70, 80, 90, or 100-fold higher compared to the reference polypeptide. In some exemplary embodiments, the binding polypeptides of the composition comprising Man₅₋₉ (GlcNAc)₂ N-glycans have increased antibody-dependent cellular cytotoxicity (ADCC) activity compared to the reference polypeptide. In certain exemplary embodiments, the ADCC activity of the binding polypeptides is at least 1, 2, 3, 4, or 5-fold higher compared to the reference polypeptide. In a certain exemplary embodiment, the reference polypeptide has a wildtype (WT) Fc domain. In another exemplary embodiment, the reference polypeptide has not been produced by culturing a cell that expresses the reference polypeptide in the presence of kifunensine.

In certain exemplary embodiments, the Fc domain of the binding polypeptides in the composition comprises an aspartic acid (D) at amino acid position 239.

In other exemplary embodiments, the Fc domain of the binding polypeptides in the composition comprise a glutamic acid (E) at amino acid position 332.

In still other exemplary embodiments, the Fc domain of the binding polypeptides in the composition comprises an aspartic acid (D) at amino acid position 239 and a glutamic acid (E) at amino acid position 332.

In other exemplary embodiments, the Fc domain of the binding polypeptides in the composition comprises an aspartic acid (D) at amino acid position 267.

In still other exemplary embodiments, the Fc domain of the binding polypeptides in the composition comprises an aspartic acid (D) at amino acid position 268.

In certain exemplary embodiments, the Fc domain of the binding polypeptides in the composition comprises a glutamic acid (E) at amino acid position 268.

In some exemplary embodiments, the Fc domain of the binding polypeptides in the composition comprises an alanine (A) at amino acid position 298.

In still other exemplary embodiments, the Fc domain of the binding polypeptides in the composition comprises an aspartic acid (D) at amino acid position 239 and an alanine (A) at amino acid position 298.

In certain exemplary embodiments, the Fc domain of the binding polypeptides in the composition comprises a cysteine (C) at amino acid position 298.

In some exemplary embodiments, the Fc domain of the binding polypeptides in the composition comprises an isoleucine (I) at amino acid position 314.

In still other exemplary embodiments, the Fc domain of the binding polypeptides in the composition comprises a methionine (M) at amino acid position 314.

In further exemplary embodiments, the Fc domain of the binding polypeptides in the composition comprises a glutamine (Q) at amino acid position 314.

In other exemplary embodiments, the Fc domain of the binding polypeptides in the composition comprises a tryptophan (W) at amino acid position 314.

In still other exemplary embodiments, the Fc domain of the binding polypeptides in the composition comprises a phenylalanine (F) at amino acid position 330.

In further exemplary embodiments, the Fc domain of the binding polypeptides in the composition comprises a methionine (M) at amino acid position 330.

In certain exemplary embodiments, the Fc domain of the binding polypeptides in the composition comprises an aspartic acid (D) at amino acid position 339.

In other exemplary embodiments, the Fc domain of the binding polypeptides in the composition comprises an isoleucine (I) at amino acid position 339.

In still other exemplary embodiments, the Fc domain of the binding polypeptides in the composition comprises a proline (P) at amino acid position 339.

In certain exemplary embodiments, the Fc domain of the binding polypeptides in the composition comprises a threonine (T) at amino acid position 339.

In further exemplary embodiments, the Fc domain of the binding polypeptides in the composition comprises a phenylalanine (F) at amino acid position 373.

In additional exemplary embodiments, the Fc domain of the binding polypeptides in the composition comprises a tryptophan (W) at amino acid position 373.

In another aspect, a composition comprising a population of isolated glycosylated binding polypeptides each comprising an Fc domain comprising an N-glycan,
wherein the Fc domain further comprises a mutation that increases binding to an Fc receptor,
wherein the composition comprises at least 50% Man₅₋₉ (GlcNAc)₂ N-glycans by molar ratio, relative to all N-glycans, and
wherein the Fc domain further comprises a cysteine (C) at amino acid position 292 and a cysteine (C) at amino acid position 302, according to EU numbering, is provided.

In certain exemplary embodiments, Man₈ and Man₉ together are the major species of Man₅₋₉(GlcNAc)₂ N-glycans in the composition.

In some exemplary embodiments, the composition comprises greater than 70%, 75%, 80%, 85%, 90%, or 95% Man₉(GlcNAc)₂ N-glycans by molar ratio, relative to all N-glycans.

In still other exemplary embodiments, the composition comprises at least 97% Man₉(GlcNAc)₂ N-glycans by molar ratio, relative to all N-glycans.

In certain exemplary embodiments, at least 80% of the N-glycans by molar ratio, relative to all N-glycans in the composition are afucosylated.

In other exemplary embodiments, the binding polypeptides of the composition are produced by culturing cells that express the binding polypeptides in the presence of a mannosidase inhibitor. In some embodiments, the mannosidase inhibitor is kifunensine. In certain embodiments, the concentration of kifunensine is from about 60 ng/ml to about 2500 ng/ml. In one exemplary embodiment, the concentration of kifunensine is about 2000 ng/ml.

In certain exemplary embodiments, the binding polypeptides of the composition comprising Man₅₋₉(GlcNAc)₂ N-glycans have increased affinity for binding to an Fcγ receptor compared to a reference polypeptide that does not comprise Man₅₋₉(GlcNAc)₂ N-glycans but is otherwise identical. In exemplary embodiments, the Fcγ receptor is human FcγRIIIa. In particular exemplary embodiments, the binding polypeptides of the composition comprising Man₅₋₉ (GlcNAc)₂ N-glycans have increased affinity for binding to human FcγRIIIa of at least 2, 3, 4, 5, 10, 20, 30, 40, 50, 60, 70, 80, 90, or 100-fold higher compared to the reference polypeptide. In some exemplary embodiments, the binding polypeptides comprising Man₅₋₉(GlcNAc)₂ N-glycans of the composition have increased antibody-dependent cellular cytotoxicity (ADCC) activity compared to the reference polypeptide. In certain exemplary embodiments, the ADCC activity of the binding polypeptides is at least 1, 2, 3, 4, or 5-fold higher compared to the reference polypeptide. In certain exemplary embodiments, the reference polypeptide has a wildtype (WT) Fc domain.

In certain exemplary embodiments, the Fc domain of the binding polypeptides in the composition comprises an aspartic acid (D) at amino acid position 239.

In other exemplary embodiments, the Fc domain of the binding polypeptides in the composition comprises a glutamic acid (E) at amino acid position 332.

In still other exemplary embodiments, the Fc domain of the binding polypeptides in the composition comprises an aspartic acid (D) at amino acid position 239 and a glutamic acid (E) at amino acid position 332.

In other exemplary embodiments, the Fc domain of the binding polypeptides in the composition comprises an aspartic acid (D) at amino acid position 267.

In still other exemplary embodiments, the Fc domain of the binding polypeptides in the composition comprises an aspartic acid (D) at amino acid position 268.

In certain exemplary embodiments, the Fc domain of the binding polypeptides in the composition comprises a glutamic acid (E) at amino acid position 268.

In some exemplary embodiments, the Fc domain of the binding polypeptides in the composition comprises an alanine (A) at amino acid position 298.

In still other exemplary embodiments, the Fc domain of the binding polypeptides in the composition comprises an aspartic acid (D) at amino acid position 239 and an alanine (A) at amino acid position 298.

In certain exemplary embodiments, the Fc domain of the binding polypeptides comprises a cysteine (C) at amino acid position 298.

In some exemplary embodiments, the Fc domain of the binding polypeptides in the composition comprises an isoleucine (I) at amino acid position 314.

In still other exemplary embodiments, the Fc domain of the binding polypeptides in the composition comprises a methionine (M) at amino acid position 314.

In further exemplary embodiments, the Fc domain of the binding polypeptides in the composition comprises a glutamine (Q) at amino acid position 314.

In other exemplary embodiments, the Fc domain of the binding polypeptides in the composition comprises a tryptophan (W) at amino acid position 314.

In still other exemplary embodiments, the Fc domain of the binding polypeptides in the composition comprises a phenylalanine (F) at amino acid position 330.

In further exemplary embodiments, the Fc domain of the binding polypeptides in the composition comprises a methionine (M) at amino acid position 330.

In certain exemplary embodiments, the Fc domain of the binding polypeptides in the composition comprises an aspartic acid (D) at amino acid position 339.

In other exemplary embodiments, the Fc domain of the binding polypeptides in the composition comprises an isoleucine (I) at amino acid position 339.

In still other exemplary embodiments, the Fc domain of the binding polypeptides in the composition comprises a proline (P) at amino acid position 339.

In certain exemplary embodiments, the Fc domain of the binding polypeptides in the composition comprises a threonine (T) at amino acid position 339.

In further exemplary embodiments, the Fc domain of the binding polypeptides in the composition comprises a phenylalanine (F) at amino acid position 373.

In additional exemplary embodiments, the Fc domain of the binding polypeptides in the composition comprises a tryptophan (W) at amino acid position 373.

In certain exemplary embodiments, the Fc domain of the binding polypeptides further comprises an aspartic acid (D) at amino acid position 256 and a glutamine (Q) at amino acid position 307.

In additional exemplary embodiments, the binding polypeptides of the composition comprising Man₅₋₉(GlcNAc)₂ N-glycans have a Tm within 10 degrees Celsius of a reference polypeptide with a WT Fc domain. In some embodiments, the reference polypeptide with a WT Fc domain is expressed by a cell that is cultured in the absence of kifunensine and the binding polypeptides comprising Man₅₋₉(GlcNAc)₂ N-glycans are expressed by cells cultured in the presence of kifunensine. In some embodiments, the binding polypeptides comprising Man₅₋₉(GlcNAc)₂ N-glycans have a Tm within 5 degrees Celsius of a reference polypeptide with a WT Fc domain. In some embodiments, the reference polypeptide with a WT Fc domain is expressed by a cell that is cultured in the presence of kifunensine and the binding polypeptides comprising Man₅₋₉(GlcNAc)₂ N-glycans are expressed by cells cultured in the presence of a kifunensine.

In still another aspect, a composition comprising a population of isolated glycosylated binding polypeptides each comprising an Fc domain comprising an N-glycan,
wherein the Fc domain further comprises a mutation that increases binding to an Fc receptor,
wherein the composition comprises at least 50% Man₅₋₉(GlcNAc)₂ N-glycans by molar ratio, relative to all N-glycans,
and wherein the Fc domain further comprises an aspartic acid (D) at amino acid position 256 and a glutamine (Q) at amino acid position 307, according to EU numbering, is provided.

In certain exemplary embodiments, Man₈ and Man₉ together are the major species of Man₅₋₉(GlcNAc)₂ N-glycans in the composition.

In some exemplary embodiments, the composition comprises greater than 70%, 75%, 80%, 85%, 90%, or 95% Man₉(GlcNAc)₂ N-glycans by molar ratio, relative to all N-glycans.

In still other exemplary embodiments, the composition comprises at least 97% Man₉(GlcNAc)₂ N-glycans by molar ratio, relative to all N-glycans.

In certain exemplary embodiments, at least 80% of the N-glycans by molar ratio, relative to all N-glycans in the composition are afucosylated.

In other exemplary embodiments, the binding polypeptides of the composition are produced by culturing cells that express the binding polypeptides in the presence of a mannosidase inhibitor.

In certain exemplary embodiments, the binding polypeptides of the composition comprising Man₅₋₉(GlcNAc)₂ N-glycans have increased affinity for binding to an Fcγ receptor compared to a reference polypeptide that does not comprise Man₅₋₉(GlcNAc)₂ N-glycans but is otherwise identical. In exemplary embodiments, the Fcγ receptor is human FcγRIIIa. In particular exemplary embodiments, the binding polypeptides of the composition comprising Man₅₋₉ (GlcNAc)₂ N-glycans have increased affinity for binding to human FcγRIIIa of at least 2, 3, 4, 5, 10, 20, 30, 40, 50, 60, 70, 80, 90, or 100-fold higher compared to the reference polypeptide. In some exemplary embodiments, the binding polypeptides comprising Man₅₋₉(GlcNAc)₂ N-glycans of the composition have increased antibody-dependent cellular cytotoxicity (ADCC) activity compared to the reference polypeptide. In certain exemplary embodiments, the ADCC activity of the binding polypeptides is at least 1, 2, 3, 4, or 5-fold higher compared to the reference polypeptide. In certain exemplary embodiments, the reference polypeptide has a wildtype (WT) Fc domain.

In other exemplary embodiments, the binding polypeptides of the composition are produced by culturing cells that express the binding polypeptides in the presence of a mannosidase inhibitor. In some embodiments, the mannosidase inhibitor is kifunensine. In certain embodiments, the concentration of kifunensine is from about 60 ng/ml to about 2500 ng/ml. In one exemplary embodiment, the concentration of kifunensine is about 2000 ng/ml.

In certain exemplary embodiments, the Fc domain of the binding polypeptides in the composition comprises an aspartic acid (D) at amino acid position 239.

In other exemplary embodiments, the Fc domain of the binding polypeptides in the composition comprises a glutamic acid (E) at amino acid position 332.

In still other exemplary embodiments, the Fc domain of the binding polypeptides in the composition comprises an aspartic acid (D) at amino acid position 239 and a glutamic acid (E) at amino acid position 332.

In other exemplary embodiments, the Fc domain of the binding polypeptides in the composition comprises an aspartic acid (D) at amino acid position 267.

In still other exemplary embodiments, the Fc domain of the binding polypeptides in the composition comprises an aspartic acid (D) at amino acid position 268.

In certain exemplary embodiments, the Fc domain of the binding polypeptides in the composition comprises a glutamic acid (E) at amino acid position 268.

In some exemplary embodiments, the Fc domain of the binding polypeptides in the composition comprises an alanine (A) at amino acid position 298.

In still other exemplary embodiments, the Fc domain of the binding polypeptides in the composition comprises an aspartic acid (D) at amino acid position 239 and an alanine (A) at amino acid position 298.

In certain exemplary embodiments, the Fc domain of the binding polypeptides comprises a cysteine (C) at amino acid position 298.

In some exemplary embodiments, the Fc domain of the binding polypeptides in the composition comprises an isoleucine (I) at amino acid position 314.

In still other exemplary embodiments, the Fc domain of the binding polypeptides in the composition comprises a methionine (M) at amino acid position 314.

In further exemplary embodiments, the Fc domain of the binding polypeptides in the composition comprises a glutamine (Q) at amino acid position 314.

In other exemplary embodiments, the Fc domain of the binding polypeptides in the composition comprises a tryptophan (W) at amino acid position 314.

In still other exemplary embodiments, the Fc domain of the binding polypeptides in the composition comprises a phenylalanine (F) at amino acid position 330.

In further exemplary embodiments, the Fc domain of the binding polypeptides in the composition comprises a methionine (M) at amino acid position 330.

In certain exemplary embodiments, the Fc domain of the binding polypeptides in the composition comprises an aspartic acid (D) at amino acid position 339.

In other exemplary embodiments, the Fc domain of the binding polypeptides in the composition comprises an isoleucine (I) at amino acid position 339.

In still other exemplary embodiments, the Fc domain of the binding polypeptides in the composition comprises a proline (P) at amino acid position 339.

In certain exemplary embodiments, the Fc domain of the binding polypeptides in the composition comprises a threonine (T) at amino acid position 339.

In further exemplary embodiments, the Fc domain of the binding polypeptides in the composition comprises a phenylalanine (F) at amino acid position 373.

In additional exemplary embodiments, the Fc domain of the binding polypeptides in the composition comprises a tryptophan (W) at amino acid position 373.

In certain exemplary embodiments, the binding polypeptides in the composition comprising Man₅₋₉(GlcNAc)₂ N-glycans have a higher binding affinity to neonatal Fc receptor (FcRn) compared to a binding polypeptide with a WT Fc domain.

In other exemplary embodiments, the Fc domain of the binding polypeptides in the composition further comprises a cysteine (C) at amino acid position 292 and a cysteine (C) at amino acid position 302, according to EU numbering.

In certain exemplary embodiments, one or more of the binding polypeptides in the composition is an antibody. In some embodiments, the antibody is a monoclonal antibody. In some embodiments, the antibody is a chimeric, a humanized, or a human antibody. In some embodiments, the antibody is a multispecific antibody. In some embodiments, the multispecific antibody is of a format selected from the group consisting of: a DVD-Ig, a CODV based format that is optionally a CODV-Ig, a CrossMab, a CrossMab-Fab, and a Tandem Fabs. In some embodiments, the multispecific antibody is a T cell engager. In some embodiments, the multispecific antibody is a NK cell engager.

In some embodiments, one or more of the binding polypeptide comprise an immunoglobulin single variable domain (ISV).

In some embodiments, one or more of the binding polypeptides of the disclosure comprise one or more VHH.

In further exemplary embodiments, one or more of the binding polypeptides in the composition comprise an antigen binding fragment.

In still other exemplary embodiments, one or more of the binding polypeptides in the composition comprise a single chain variable region (ScFv) sequence.

In additional exemplary embodiments, one or more of the binding polypeptides in the composition comprise an IgG Fc domain. In some embodiments, the Fc domain is an IgG1 domain. In some embodiments, the Fc domain is a human Fc domain.

In still other exemplary embodiments, one or more of the binding polypeptides in the composition comprises a lysosome-targeting chimera (LYTAC).

In yet other embodiments, one or more of the binding polypeptides comprises an Fc domain that further comprises a mutation that increases binding to an Fc receptor, wherein the Fc receptor is a human FcγRIIIa receptor.

In certain embodiments, the composition is a pharmaceutical composition.

In an additional aspect, a method of making the binding polypeptides of the composition comprising culturing a cell that expresses the binding polypeptides in the presence of kifunensine, is provided. In some embodiments, the concentration of kifunensine in cell culture is about 60 ng/ml to about 2500 ng/ml. In some embodiments, the concentration of kifunensine in cell culture is about 2000 ng/ml.

In a further aspect, an isolated nucleic acid molecule comprising a nucleic acid capable of expressing one or more of the binding polypeptides of the composition, is provided. In some embodiments, a vector comprising the isolated nucleic acid molecule is provided. In some embodiments, the vector is an expression vector. In a further aspect, a host cell comprising the vector is provided.

In yet another aspect, a method of treating a disease or disorder in a subject in need thereof, comprising administering to the subject an effective amount of the pharmaceutical composition, is provided. In some embodiments, the disease or disorder is a cancer. In some embodiments, the disease or disorder is an inflammatory disease. In some embodiments, the disease or disorder is an autoimmune disease.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a schematic diagram that depicts the production of antibodies using inhibitors, such as kifunensine, that inhibit enzymes in the glycosylation pathway. This diagram shows that adding kifunensine arrests glycan processing, prevents the addition of fucose, and prevents the breakdown of the oligomannose structure.

FIG. 2A-B depicts SDS-PAGE analysis of wild-type (WT) and DE (S239D/I332E) antibodies specific for protein 1 with different glycan modifications. FIG. 2A shows the antibodies under non-reducing conditions, and FIG. 2B shows the antibodies under reducing conditions. In both FIG. 2A and FIG. 2B, the content of the lanes is as follows: lane 1: protein molecular weight marker, lane 2: WT, lane 3: hypergalactosylated WT, lane 4: WT containing oligomannose, lane 5: afucosylated WT, lane 6: DE, lane 7: hypergalactosylated DE, lane 8: DE containing oligomannose, and lane 9: afucosylated DE.

FIG. 3A-H depict the structure and MALDI spectra of N-linked glycans from antibodies specific for protein 1 with different glycan modifications. The N-linked glycans were released from antibodies with PNGase F, and analyzed using MALDI-TOF MS. The MALDI spectra are presented for WT (FIG. 3A) and DE (FIG. 3B) antibodies; hypergalactosylated WT (FIG. 3C) and DE (FIG. 3D) antibodies; oligomannose containing WT (FIG. 3E) and DE (FIG. 3F) antibodies; and afucosylated WT (FIG. 3G) and DE (FIG. 3H) antibodies.

FIG. 4A-B graphically depict FcγRIIIa binding of antibodies specific for protein 1 with different glycan modifications. The interactions of FcγRIIIa with the antibodies was measured by SPR using Biacore™. FIG. 4A depicts the receptor binding of WT and DE antibodies containing hypergalactose or oligomannose. FIG. 4B depicts the receptor binding of a fucose WT and DE antibodies.

FIG. 5A-B graphically depict ADCC activity of antibodies specific for protein 1 with different glycan modifications. The effector function of the antibodies was determined using an ADCC reporter gene assay. FIG. 5A depicts the ADCC activity of WT and DE antibodies containing hypergalactose or oligomannose. FIG. 5B depicts the ADCC activity of a fucose WT and DE antibodies.

FIG. 6A-B graphically depicts the correlation of various percentages of afucosylated glycans in DE antibodies specific for protein 1 with FcγRIIIa binding and ADCC. FIG. 6A depicts FcγRIIIa binding of the DE antibody with different percentages of afucosylated glycans. FIG. 6B depicts ADCC of the DE antibody with different percentages of afucosylated glycans.

FIG. 7A-B depicts SDS-PAGE of Fc variants of antibodies specific for protein 2 with and without kifunensine treatment. FIG. 7A depicts non-reducing conditions. FIG. 7B depicts reducing conditions.

FIG. 8A-F graphically depicts MALDI-TOF glycan analysis of variants of antibodies specific for protein 2 with or without kifunensine treatment. FIG. 8A illustrates that the wildtype antibody has predominately G0F-Gn, G0F, and G1F glycans without kifunensine treatment and Man8 and Man9 glycans with kifunensine treatment. FIG. 8B illustrates the S298A antibody has predominately G0F and G1F glycans without kifunensine treatment and Man9 glycans with kifunensine treatment.

FIG. 8C illustrates the S239D antibody has predominately G0F-Gn, G0F, and G1F glycans without kifunensine treatment and Man8 and Man9 glycans with kifunensine treatment. FIG. 8D illustrates the S239D/S298A antibody has predominately G0F-Gn, G0F, and G1F glycans without kifunensine treatment and Man9 glycans with kifunensine treatment. FIG. 8E illustrates the I332E antibody has predominately G0F-Gn, G0F, and G1F glycans without kifunensine treatment and Man8 and Man9 glycans with kifunensine treatment. FIG. 8F illustrates the S239D/I332E antibody has predominately G0F-Gn, G0F, and G1F glycans without kifunensine treatment and Man8 and Man9 glycans with kifunensine treatment.

FIG. 9A-G depicts human FcγRIIIa binding affinity results for various Fc variants of antibodies specific for protein 2 with and without kifunensine treatment. FIG. 9A is a table that includes the measured binding affinity metrics. These results show that kifunensine treatment increases affinity to hFcγRIIIa 2.4- to 7-fold for all Fc variants. FIG. 9B depicts sensorgams of binding for the WT antibody with and without kifunensine treatment. FIG. 9C depicts sensorgams of binding for the S239D (D) antibody with and without kifunensine treatment. FIG. 9D depicts sensorgams of binding for the S239D/S298A (DA) antibody with and without kifunensine treatment. FIG. 9E depicts sensorgams of binding for the S298A (A) antibody with and without kifunensine treatment. FIG. 9F depicts sensorgams of binding for the I332E antibody with and without kifunensine treatment. FIG. 9G depicts sensorgams of binding for the S239D/I332E (DE) antibody with and without kifunensine treatment.

FIG. 10 graphically depicts nanoDSF analysis of the tm of antibodies specific for protein 2, including WT without kifunensine, WT with kifunensine, S239D/I332E without kifunensine, and S239D/S298A with kifunensine. All variants had a lower Tm when compared to WT without kifunensine.

FIG. 11A-F graphically depicts mass spectrometry glycan analysis of various antibodies specific for protein 3 with or without kifunensine treatment. FIG. 11A depicts WT antibodies specific for protein 3 with or without kifunensine treatment.

FIG. 11B depicts LS antibodies specific for protein 3 with or without kifunensine treatment. FIG. 11C depicts YTE antibodies specific for protein 3 with or without kifunensine treatment. FIG. 11D depicts YD antibodies specific for protein 3 with or without kifunensine treatment. FIG. 11E depicts DQ antibodies specific for protein 3 with or without kifunensine treatment. FIG. 11F depicts DW antibodies specific for protein 3 with or without kifunensine treatment. All kifunesine treated antibodies have an oligomannose content of >97% Man₉(GlcNAc)₂. All untreated antibodies are >80% afucosylated.

FIG. 12 graphically depicts FcγRIIIa binding affinity response for WT, DQ, DW, LS, YD, and YTE antibodies specific for protein 3 with and without kifunensine treatment. Enhanced FcγRIIIa binding was observed for all variants expressed with kifunensine.

FIG. 13A-C graphically depicts human FcRn binding at pH 6.0 (FIG. 13A) and pH 7.4 (FIG. 13B) for various antibodies specific for protein 3. Both FIG. 13A (pH 6.0) and FIG. 13B (pH 7.4) depict binding results for WT, LS, YTE, DQ, DW, and YD antibodies specific for protein 3 with and without kifunensine treatment. FIG. 13C depicts a scatter plot of the results at pH 6.0. At pH 6.0 no significant changes in the on or off binding rates were observed. DQ, DW and YD all have faster on and off binding rates compared to LS. At pH 7.0, slightly reduced human FcRn binding response was observed in the kifunesine treated samples.

FIG. 14 graphically depicts thermal stability as determined by DSF for WT, LS, YTE, DQ, DW, and YD antibodies specific for protein 3 with and without kifunensine treatment. The curves shown in solid black are without kifunensine treatment and the curves shown in the dotted line are with kifunensine treatment. Kifunensine treatment destabilizes every antibody variant a further 4-8° C. (versus the Fc mutation alone.) DW with kifunensine treatment shows a 16° C. decrease in thermal stability compared to the WT.

FIG. 15 graphically depicts FcRn affinity determined by chromatography for WT, LS, YTE, DQ, DW, and YD antibodies specific for protein 3 with and without kifunensine treatment. The curves shown in solid black are without kifunensine treatment and the curves shown in the dotted line are with kifunensine treatment. The kifunensine treated samples show a similar pH elution profile as the untreated samples. This data supports the FcRn binding results showing little effect on overall binding affinity upon treatment with kifunensine.

FIG. 16 is a table providing metrics related to human FcγRIIIa binding affinity of human IgG1 antibodies specific for protein 4 including various combinations of S239D (D), S239D/S298A (DA), S239D/I332E (DE), R292C/V302C (SEFL2.2), T256D/T307Q (DQ) and M428L/N434S (LS) with and without kifunensine treatment. These results show kifunensine treatment increases affinity to hFcγRIIIa 1.6- to 7.7-fold for all Fc variants tested. DE has the highest affinity with kifunensine treatment. R292C/V302C, DQ, DQ+R292C/V302C, and LS retained binding affinity similar to the WT antibody.

FIG. 17A-E depict sensorgrams of human FcγRIIIa binding affinity of the following antibodies specific for protein 4: WT (FIG. 17A), DE (FIG. 17B), DA with kifunensine treatment (FIG. 17C), DQ+D+R292C/V302C (SEFL2.2) with kifunensine treatment (FIG. 17D), and DQ+DA+R292C/V302C (SEFL2.2) with kifunensine treatment (FIG. 17E). All variants tested showed stronger binding affinity than the WT antibody.

FIG. 18 depicts SDS-PAGE for antibodies specific for protein 5 under reducing and non-reducing conditions. For both the reducing and non-reducing gels, the lanes are as follows: lane 1: DA+kifunensine, lane 2: DE+R292C/V302C, lane 3: afucosylated, and lane 4: WT.

FIG. 19A-D graphically depicts MALDI-TOF glycan analysis of antibodies specific for protein 5. FIG. 19A depicts results for the WT antibody. The major glycans were determined to be G0F and G1F. The WT antibodies were also determined to be 95.1% fucosylated and 4.9% afucosylated. FIG. 19B depicts results for the DE+R292C/V302C antibody. The major glycans were determined to be G0F and G1F. FIG. 19C depicts results for the DA+kifunensine antibody. The major glycans were determined to be Man₉(GlcNAc)₂ and Man₈(GlcNAc)₂. FIG. 19D depicts results for the afucosylated antibody. The major glycans were determined to be G0.

FIG. 20A-D depicts sensorgrams of binding analysis of various antibodies to protein 5. FIG. 20A depicts the WT antibody. FIG. 20B depicts the DA+kifunensine antibody. FIG. 20C depicts the DE+R292C/V302C (disulfide) antibody. FIG. 20D depicts the afucosylated antibody. All antibodies had similar binding affinity to protein 5.

FIG. 21A-D depict sensorgams of binding affinity to human FcγRIIIa for various antibodies specific for protein 5. FIG. 21A depicts the WT antibody. FIG. 21B depicts the DA+kifunensine antibody. FIG. 21C depicts the DE+R292C/V302C (disulfide) antibody. FIG. 21D depicts the afucosylated antibody. All variants showed a higher binding affinity for human FcγRIIIa over WT.

FIG. 22A-F depict sensorgrams of human FcγRIIIa binding affinity for the following antibodies: WT (FIG. 22A), WT with kifunensine treatment (FIG. 22B), S298A (FIG. 22C), S298A with kifunensine treatment (FIG. 22D), H268D (FIG. 22E), and H268D with kifunesine treatment (FIG. 22F). All variants showed a higher binding affinity for human FcγRIIIa over WT.

FIG. 23A-B present the numerical values for human FcγRIIIa binding affinity without kifunesine (FIG. 23A) and with kifunesine (FIG. 23B). Values for WT in FIG. 23A and FIG. 23B are shown in bold. These figures show that for some variants tested, binding affinity for human FcγRIIIa is increased versus WT.

FIG. 24 presents the numerical values for human FcγRIIIa binding affinity with and without kifunesine, as well as the Tm values for the Fc variants tested and WT. This figure shows that for some variants tested, binding affinity for human FcγRIIIa is increased versus WT.

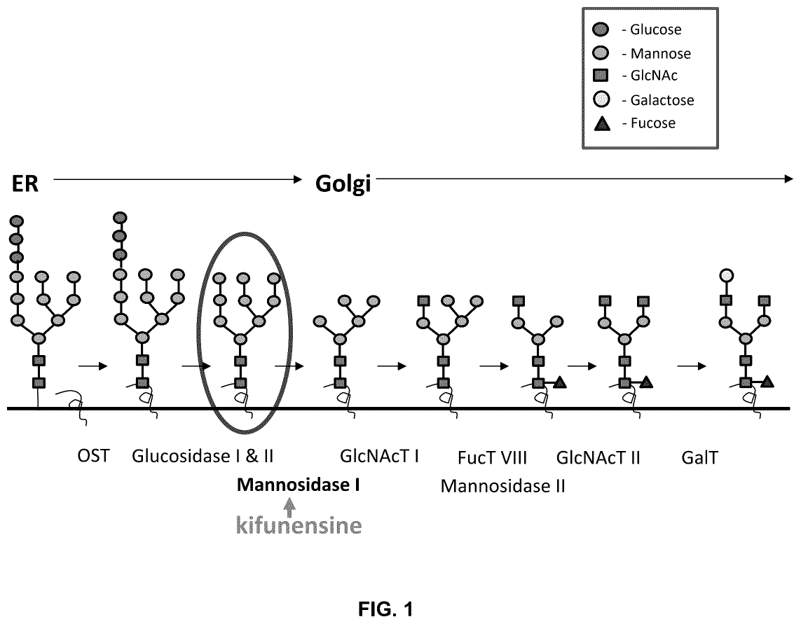

### DETAILED DESCRIPTION

The current disclosure provides novel glycoengineered Fc domain variants (e.g., binding polypeptides comprising Fc domain variants) comprising mannose-rich glycans (e.g., oligomannose-type N-glycans). In exemplary embodiments, the Fc domain variants contain both an Fc mutation that enhances binding to an Fc receptor as well as one or more oligomannose-type N-glycans conjugated to the Fc region. In some exemplary embodiments, the Fc variants are produced in host cells cultured in the presence of a mannosidase inhibitor (e.g., the α-mannosidase I inhibitor kifunensine). In exemplary embodiments, the invention provides a composition comprising a population of Fc variant polypeptides wherein at least 50% (e.g., at least 50%, at least 60%, at least 70%, at least 80%, at least 90%, at least 95%) of the Fc variant polypeptides comprise an oligomannose-type N-glycan. In exemplary embodiments, the predominant oligomannose-type N-glycan is Man₈(GlcNAc)₂ or Man₉(GlcNAc)₂.

In certain embodiments, the glycoengineered Fc domain variants of the disclosure have improved antibody-dependent cellular cytotoxicity (ADCC) activity as compared to an Fc domain variant that lacks an oligomannose-type N-glycan but is otherwise identical to the glycoengineered Fc domain variant. In some exemplary embodiments, the glycoengineered Fc domain variants comprise Fc mutations which confer improved thermal stability. The present disclosure also provides nucleic acids encoding glycoengineered Fc domain variants, recombinant expression vectors and host cells for making glycoengineered Fc domain variants, and pharmaceutical compositions comprising the isolated glycoengineered Fc domain variants. Methods of using the binding polypeptides comprising glycoengineered Fc domain variants disclosed herein to treat various diseases are also provided. The glycoengineered Fc domain variants disclosed herein are useful for various therapies in which Fc-directed killing of target cells is desirable.

### Definitions

It is to be understood that the methods described in this disclosure are not limited to particular methods and experimental conditions disclosed herein as such methods and conditions may vary. It is also to be understood that the terminology used herein is for the purpose of describing particular embodiments only and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2^{nd} edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The term “polypeptide” refers to any polymeric chain of amino acids and encompasses native or artificial proteins, polypeptide analogs or variants of a protein sequence, or fragments thereof, unless otherwise contradicted by context. A polypeptide may be monomeric or polymeric. For an antigenic polypeptide, a fragment of a polypeptide optionally contains at least one contiguous or nonlinear epitope of a polypeptide. The precise boundaries of the at least one epitope fragment can be confirmed using ordinary skill in the art. A polypeptide fragment comprises at least about 5 contiguous amino acids, at least about 10 contiguous amino acids, at least about 15 contiguous amino acids, or at least about 20 contiguous amino acids, for example.

The term “isolated protein” or “isolated polypeptide” refer to a protein or polypeptide that by virtue of its origin or source of derivation is not associated with naturally associated components that accompany it in its native state; is substantially free of other proteins from the same species; is expressed by a cell from a different species; or does not occur in nature. Thus, a protein or polypeptide that is chemically synthesized or synthesized in a cellular system different from the cell from which it naturally originates will be “isolated” from its naturally associated components. A protein or polypeptide may also be rendered substantially free of naturally associated components by isolation using protein purification techniques well known in the art.

As used herein, the term “binding protein” or “binding polypeptide” shall refer to a protein or polypeptide (e.g., an antibody or fragment thereof) that contains at least one binding site which is responsible for selectively binding to a target antigen of interest (e.g., a human antigen). Exemplary binding sites include an antibody variable domain, a ligand binding site of a receptor, or a receptor binding site of a ligand. In certain aspects, the binding proteins or binding polypeptides comprise multiple (e.g., two, three, four, or more) binding sites. In certain aspects, the binding protein or binding polypeptide is not a therapeutic enzyme.

As used herein, the term “native residue” shall refer to an amino acid residue that occurs naturally at a particular amino acid position of a binding polypeptide (e.g., an antibody or fragment thereof) and which has not been modified, introduced, or altered by the hand of man. As used herein, the term “altered binding protein,” “altered binding polypeptide,” “modified binding protein” or “modified binding polypeptide” shall refer to binding polypeptides and/or binding proteins (e.g., an antibody or fragment thereof) comprising at least one amino acid substitution, deletion and/or addition relative to the native (i.e., wild-type) amino acid sequence, and/or a mutation that results in altered glycosylation (e.g., hyperglycosylation, hypoglycosylation and/or aglycosylation) at one or more amino acid positions relative to the native (i.e., wild-type) amino acid sequence.

The term “ligand” refers to any substance capable of binding, or of being bound, to another substance. Similarly, the term “antigen” refers to any substance to which an antibody may be generated. Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein.

Antigens/ligands may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

The term “specifically binds” as used herein, refers to the ability of an antibody or an antigen-binding fragment thereof to bind to an antigen with a dissociation constant (K_{D}) of at most about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or less, and/or to bind to an antigen with an affinity that is at least two-fold greater than its affinity for a nonspecific antigen. Specific binding of an antibody can be to a target antigen through the CDR sequences. An antibody can also specifically bind to FcRs, such as FcRn or FcγRIIIa through the Fc region.

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the Biacore system (Cytiva Life Sciences, Marlborough, MA) or Carterra LSA platform (Carterra, Salt Lake City, UT). Surface plasmon analysis can also be performed by immobilizing the analyte (binding protein on a biosensor matrix) and presenting the ligand (target antigen). The term “K_{D}” as used herein refers to the dissociation constant of the interaction between a particular binding protein and a target antigen.

The term “immunoglobulin domain” as used herein can refer to an immunoglobulin A, an immunoglobulin D, an immunoglobulin E, an immunoglobulin G, or an immunoglobulin M. The immunoglobulin domain can be an immunoglobulin heavy chain region or fragment thereof. In some instances, the immunoglobulin domain is from an antibody (e.g., a mammalian antibody, a recombinant antibody, a chimeric antibody, an engineered antibody, a human antibody, a humanized antibody) or an antigen binding fragment thereof.

As used herein, the term “antibody” refers to such assemblies (e.g., intact antibody molecules, antibody fragments, or variants thereof) which have significant known specific immunoreactive activity to an antigen of interest (e.g., a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs”, as defined herein.

The regions of an immunoglobulin heavy or light chain may be defined as “constant” (C) region or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are well known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains”. Constant domains on the heavy chain (e.g., hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “VL region domains or “VL domains”. Variable domains on the heavy chain are referred to interchangeably as “heavy chain variable region domains”, “VH region domains” or “VH domains”.

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy chain constant region is via the EU index as set forth in Kabat (Kabat, Sequences of Proteins of Immunological Interest, National Institutes of Health, Bethesda, MD, 1987 and 1991).

The term “EU index” refers to the EU numbering convention for the constant regions of an antibody, as described in Edelman G M et al. (1969), Proc. Natl. Acad. USA, 63, 78-85 and Kabat et al., Sequences of Proteins of Immunological Interest, U.S. Dept. Health and Human Services, 5th edition, 1991, each of which is herein incorporated by reference in its entirety. Unless otherwise stated, all antibody Fc region numbering employed herein corresponds to the EU numbering scheme, as described in Edelman G M et al. (1969), Proc. Natl. Acad. USA, vol. 63 (1): 78-85.

As used herein, the term “VH domain” includes the amino terminal variable domain of an immunoglobulin heavy chain, and the term “VL domain” includes the amino terminal variable domain of an immunoglobulin light chain.

As used herein, the term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the VH domain and amino terminal to the hinge region of an immunoglobulin heavy chain molecule and does not form a part of the Fc region of an immunoglobulin heavy chain.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K H et al. (1998), J. Immunol., vol. 161:4083-90).

As used herein, the term “CH2 domain” includes the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule. In one embodiment, a binding polypeptide of the current disclosure comprises a CH2 domain derived from an IgG1 molecule (e.g., a human IgG1 molecule).

As used herein, the term “CH3 domain” includes the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from CH3 domain to form the C-terminal portion of the molecule (e.g., the CH4 domain in the μ chain of IgM and the e chain of IgE). In one embodiment, a binding polypeptide of the current disclosure comprises a CH3 domain derived from an IgG1 molecule (e.g., a human IgG1 molecule).

As used herein, the term “CL domain” includes the constant region domain of an immunoglobulin light chain that extends, e.g., from about Kabat position 107A-216. The CL domain is adjacent to the VL domain. In one embodiment, a binding polypeptide of the current disclosure comprises a CL domain derived from a kappa light chain (e.g., a human kappa light chain).

As indicated above, the variable regions of an antibody allow it to selectively recognize and specifically bind epitopes on antigens. That is, the VL domain and VH domain of an antibody combine to form the variable region (Fv) that defines a three-dimensional antigen binding site. This quaternary antibody structure forms the antigen binding site present at the end of each arm of the Y. More specifically, the antigen binding site is defined by three complementary determining regions (CDRs) on each of the heavy and light chain variable regions. As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one antibody to another. The altered antibodies of the current disclosure comprise at least one antigen binding site.

In certain embodiments, binding polypeptides of the current disclosure comprise at least two antigen binding domains that provide for the association of the binding polypeptide with the selected antigen. The antigen binding domains need not be derived from the same immunoglobulin molecule. In this regard, the variable region may or be derived from any type of animal that can be induced to mount a humoral response and generate immunoglobulins against the desired antigen. As such, the variable region of a binding polypeptide may be, for example, of mammalian origin e.g., may be human, murine, rat, goat, sheep, non-human primate (such as cynomolgus monkeys, macaques, etc.), lupine, or camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by interchain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

Exemplary binding polypeptides include antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen).

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen). The subject binding polypeptides typically has at least one binding site specific for a human antigen molecule.

The term “specificity” refers to the ability to specifically bind (e.g., immunoreact with) a given target antigen (e.g., a human target antigen). A binding polypeptide may be monospecific and contain one or more binding sites which specifically bind a target or a polypeptide may be multispecific and contain two or more binding sites which specifically bind the same or different targets. In certain embodiments, a binding polypeptide is specific for two different (e.g., non-overlapping) portions of the same target. In certain embodiments, a binding polypeptide is specific for more than one target. Exemplary binding polypeptides (e.g., antibodies) which comprise antigen binding sites that bind to antigens expressed on tumor cells are known in the art and one or more CDRs from such antibodies can be included in an antibody as described herein.

The term “antigen” or “target antigen” as used herein refers to a molecule or a portion of a molecule that is capable of being bound by the binding site of a binding polypeptide. A target antigen may have one or more epitopes.

“Effector function” as used herein is a biochemical event that results from the interaction of an antibody Fc region with an Fc receptor or ligand. A “functional Fc region” possesses an “effector function” of a native sequence Fc region. Exemplary “effector functions” include antibody-dependent cell-mediated cytotoxicity (ADCC) or antibody-dependent cell-mediated phagocytosis (ADCP).

“ADCC activity” as used herein refers to the ability of a binding polypeptide to elicit an ADCC reaction. ADCC is a cell-mediated reaction in which antigen-nonspecific cytotoxic cells that express FcRs (e.g., natural killer (NK) cells, neutrophils, and macrophages) recognize binding polypeptide bound to the surface of a target cell and subsequently cause lysis of (i.e., “kill”) the target cell. The primary mediator cells are natural killer (NK) cells. NK cells express FcγRIII only, with FcγRIIIb being an activating receptor and FcγRIIIb an inhibiting one; monocytes express FcγRI, FcγRII and FcγRIII (Ravetch et al. (1991), Annu. Rev. Immunol., 9:457-92).

The term “about” or “approximately” means within about 20%, such as within about 10%, within about 5%, or within about 1% or less of a given value or range.

As used herein, “administer” or “administration” refers to the act of injecting or otherwise physically delivering a substance as it exists outside the body (e.g., an isolated binding polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment,” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

As used herein, the terms “N-glycans” or “N-linked glycans” refer to glycans that are attached at an amide nitrogen of an asparagine or an arginine residue in a protein via an N-acetylglucosamine residue. In certain embodiments, the binding polypeptides featured in the present disclosure employ glycans that are “N-linked” via an asparagine residue to a glycosylation site in the polypeptide backbone of the binding polypeptide. The glycosylation site may be a native or engineered glycosylation site. These “N-linked glycosylation sites” occur in the peptide primary structure containing, for example, the amino acid sequence asparagine-X-serine/threonine, where X is any amino acid residue except proline and aspartic acid. Such N-Glycans are fully described in, for example, Drickamer K, Taylor M E (2006). Introduction to Glycobiology, 2nd ed., which is incorporated herein by reference in its entirety.

As used herein, the term “glycoengineering” refers to any art-recognized method for altering the glycoform profile of a binding protein composition to generate a “modified glycan.” In certain embodiments, glycoengineered binding proteins and/or binding polypeptides and methods of making glycoengineered binding proteins and/or binding polypeptides are provided.

As used herein the terms “G0 glycoform” or “G0,” “G1 glycoform” or “G1,” and “G2 glycoform” or “G2” refer to N-Glycan glycoforms that have zero, one or two terminal galactose residues respectively. These terms include G0, G1, and G2 glycoforms that are fucosylated or comprise a bisecting N-acetylglucosamine residue.

As used herein the terms “G0F glycoform,” “G1F glycoform,” “G2F glycoform,” refer to “G0 glycoform,” “G1 glycoform,” and “G2 glycoform” that are fucosylated, respectively.

As used herein, the term “oligomannose-Type N-glycans” or “oligomannose glycans” refers to any one or a combination of the following mannose-rich structures Man₅(GlcNAc)₂, Man₆(GlcNAc)₂, Man₇(GlcNAc)₂, Man₈(GlcNAc)₂, and Man₉(GlcNAc)₂. See Schachter et al. “Mannose Oligosaccharide”, 4.06.3.3.1, Comprehensive Glycoscience, 2007. As used herein, Man₅ refers to the structure Man₅(GlcNAc)₂; Man₆ refers to the structure Man₆(GlcNAc)₂; Man₇ refers to the structure Man₇(GlcNAc)₂; Man₈ refers to the structure Man₈(GlcNAc)₂; and Man₉ refers to the structure Man₉(GlcNAc)₂. In certain exemplary embodiments, the oligomannose glycan is Man₈(GlcNAc)₂ or Man₉(GlcNAc)₂.

As defined herein, the phrase “lysosome-targeting chimera” or “LYTAC” refers to a bifunctional molecule that comprises a region capable of binding a cell surface lysosome targeting receptor and a region capable of binding an extracellular domain of a target protein, including, but not limited to, secreted extracellular proteins and the extracellular domain of a membrane-bound protein. Accordingly, LYTACs are useful alternatives to PROTACs described above when the target protein of interest is not intracellular. Additional LYTAC disclosure and exemplary LYTACs are described in Banik S M et al. (2019), ChemRxiv., Banik S M et al. (2020), Nature, vol. 584 (7820): 291-297, WO2015/143091, and WO2020/132100, each of which is incorporated herein by reference. As used herein, the portion of the LYTAC that is capable of binding an extracellular domain of a target protein corresponds to the antigen-binding protein or fragment thereof of the disclosure.

### Oligomannose-Type N-Glycans

In exemplary embodiments, disclosed herein are binding polypeptides that contain oligomannose-type N-glycans. In other exemplary embodiments, the oligomannose-type N-glycans are the result of culturing cells engineered to express binding polypeptides in the presence of a mannosidase inhibitor (e.g., the α-mannosidase I inhibitor kifunensine or its derivatives or functional homologs). In these embodiments, the treatment of cells with the mannosidase inhibitor results in the production of binding polypeptides carrying oligomannose-type N-glycans while the formation of complex-type N-glycans is prevented.

In other embodiments, a cell engineered to express a binding polypeptide may be deficient in one or more glycosidases required for early-stage processing of N-glycans. In some embodiments, the culture conditions may be such that the activity of one or more of these glycosidases is inhibited. As a result of one or both of these conditions, oligosaccharide synthesis is shifted toward oligomannose-type species. For example, the cell may be deficient in one or more glycosidases selected from the group consisting of α-glucosidase I, α-glucosidase II, and α-mannosidase I. Cells deficient in a glycosidase of interest can be engineered using methods as described in e.g., Tymms et al., Gene Knockout Protocols (Methods in Molecular Biology), Humana Press, 1st ed., 2001; and in Joyner, Gene Targeting: A Practical Approach, Oxford University Press, 2nd ed., 2000. For instance, glycosidase-deficient cells can be engineered using lectin selection. See Stanley P et al. (1975), Proc. Natl. Acad. USA, vol. 72 (9): 3323-3327.

In some embodiments, binding polypeptides comprising oligomannose-type N-glycans may be produced by chemical linking of an unglycosylated antibody or Fc fusion protein and a separately synthesized oligosaccharide moiety.

In some embodiments, cells are engineered to not express one or more glycosidases selected from the group consisting of α-glucosidase I, α-glucosidase II, and α-mannosidase I. In one embodiment, the glycosidase gene can be disrupted by targeted mutagenesis. In some embodiments, targeted mutagenesis can be achieved by, for example, targeting a CRISPR (clustered regularly interspaced short palindromic repeats) site in the glycosidase gene. In some embodiments, one or more expression vectors encoding at least a targeting RNA and a polynucleotide sequence encoding a CRISPR-associated nuclease, such as Cas9 is used to engineer a cell to not express a glycosidase gene.

In still other embodiments, a cell engineered to express a binding polypeptide may be contacted with an inhibitor of one or more glycosidases selected from the group consisting of α-glucosidase I, α-glucosidase II, and α-mannosidase I. In some embodiments, inhibitors of these enzymes may be, for example, small molecules or small interfering RNAs (siRNAs). siRNAs are short (20-25 nt) double stranded RNAs that inhibit a glycosidase of interest via post-transcriptional gene silencing. A glycosidase-specific siRNA may be prepared and used as described in U.S. Pat. No. 6,506,559 and/or using other suitable methods. See Appasani, RNA Interference Technology From Basic Science to Drug Development, Cambridge University Press, 1st ed., 2005; and Uei-Ti K et al. (2004), Nucleic Acids Res., vol. 32 (3): 936-948. Examples of small molecule α-glucosidase I inhibitors include castanospermine (see Pan Y T et al. (1983), Biochemistry, vol. 22 (16): 3975-3984), deoxynojirimycin (known as “DNJ”; Hettkamp H et al. (1984), Eur. J. Biochem., vol. 142:85-90) and N-alkyl and N-alkenyl derivatives thereof (e.g., N-butyl-DNJ); 2,5-dihydromethil-3,4-dihydroxypyrrolidine (known as “DMDP”; see Elbein A D et al. (1984), J. Biol. Chem., vol. 259 (2): 12409-12413); and australine (see Molyneux R J et al. (1988), J. Nat. Prod., vol. 51:1198-1206). Examples of small molecule α-glucosidase II inhibitors include DNJ and N-alkyl and N-alkenyl derivatives thereof as well as MDL 25637. See Hettkamp H et al. (1984), Eur. J. Biochem., vol. 142:85-90 and Kaushal G P, et al. (1988), J. Biol. Chem., vol. 263 (33): 17278-17283. Examples of small molecule α-mannosidase I inhibitors include deoxymannojirimycin (DMJ) (see Legler G and Julick E (1984), Carbohydr. Res., vol. 128 (1): 61-72) and derivatives thereof (e.g., N-methyl derivative as described in Bosch J V et al. (1985), Virology, vol. 143 (1): 342-346), 1,4-dideoxy-1,4-imino-D-mannitol (DIM) (see Fleet et al. (1984), J. Chem. Soc. Chem. Commun., vol. 1240-1241 and Palmarzyk G et al. (1985), Arch. Biochem. Biophys., vol. 243:35-45), and kifunensine (see Elbein A D et al. (1990), J. Biol. Chem., vol. 265:15599-15605.)

In an exemplary embodiment, cells engineered to express a binding polypeptide are cultured in the presence of the α-mannosidase I inhibitor kifunensine. In certain embodiments, kifunensine may be used at a concentration of 0.01 to 100 μg/ml, 0.01 to 75 μg/ml, 0.01 to 50 μg/ml 0.01 to 40 μg/ml, 0.01 to 30 μg/ml, 0.01 to 20 μg/ml, 0.1 to 10 μg/ml, 0.1 to 2.0 μg/ml, or 1 to 0.5 μg/ml for a period of at least 12, 24, 48, 72 hours or 4, 7, 10, 20 days or longer, or continuously. In an exemplary embodiment, CHO or hybridoma cells are incubated with about 0.5-10 μg/ml kifunensine for over 10 days. In an exemplary embodiment, the kifunensine is used at a concentration of 60 ng/ml to about 2500 ng/ml. In a further exemplary embodiment, the kifunensine is used at a concentration of 2000 ng/ml.

In still other embodiments, the oligomannose-type N-glycans on the binding polypeptides disclosed herein comprise one or more oligomannose-type oligosaccharides selected from the group consisting of Man₉(GlcNAc)₂, Man₈(GlcNAc)₂, Man₇(GlcNAc)₂, Man₆(GlcNAc)₂, and Man₅(GlcNAc)₂.

In other exemplary embodiments, compositions produced by the methods disclosed herein contain at least 20%, 25%, 30%, 35%, 40%, 45%, 50%, 55%, 60%, 65%, 70%, 75%, 80%, 85%, 90%, 95%, or 99% or more (by molar ratio relative to all N-glycans) oligomannose-type glycans Man₅₋₉ (GlcNAc)₂. In some embodiments, a composition comprising a population of isolated glycosylated binding polypeptides each comprising an Fc domain comprising an N-glycan is provided, wherein the composition comprises at least 50% Man₅₋₉(GlcNAc)₂ N-glycans by molar ratio, relative to all N-glycans.

In another exemplary embodiment, the binding polypeptides disclosed herein comprise Man₈ and Man₉ N-glycans as the major species of N-glycans. In still another exemplary embodiment, a composition comprising a population of isolated glycosylated binding polypeptides each comprising an Fc domain comprising an N-glycan is provided, wherein the composition comprises at least 50% Man₅₋₉(GlcNAc)₂ N-glycans by molar ratio, relative to all N-glycans, and Man₈ and Man₉ containing N-glycans together are the major species.

In an embodiment, the binding polypeptides disclosed herein contain predominantly Man₉(GlcNAc)₂ N-glycans. In some embodiments, a composition comprising a population of isolated glycosylated binding polypeptides each comprising an Fc domain comprising an N-glycan is provided, wherein the composition comprises at least 40%, 45%, 50%, 55%, 60%, 65%, 70%, 75%, 80%, 85%, 90%, 95%, 96%, 97%, or 99% Man₅₋₉(GlcNAc)₂ N-glycans by molar ratio, relative to all N-glycans. In an exemplary embodiment, a composition comprising a population of isolated glycosylated binding polypeptides each comprising an Fc domain comprising an N-glycan is provided, wherein the composition comprises at least 97% Man₅₋₉(GlcNAc)₂ N-glycans by molar ratio, relative to all N-glycans.

In some embodiments, the binding polypeptides of the disclosed herein contain diminishing or undetectable amounts of the oligomannose-type N-glycans Man₈(GlcNAc)₂, Man₇ (GlcNAc)₂, Man₆ (GlcNAc)₂, and Man₅ (GlcNAc)₂, while containing minor (e.g., less than 10% relative to all N-glycans) or undetectable amounts of complex type N-glycans (such as, e.g., G0, C1, G2, G0F, G1F, G2F, and G0F-Gn).

In some embodiments, the Man₅₋₉(GlcNAc)₂ in the compositions of the disclosure are substantially afucosylated (i.e., afucosylated or nonfucosylated), i.e., they contain less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (by molar ratio, relative to all N-glycans) or less fucose. In some embodiments, the compositions contain less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (by molar ratio, relative to all N-glycans) or less Man₅(GlcNAc)₂ and/or Man₆(GlcNAc)₂ N-glycans. In some embodiments, the compositions contain minor (i.e., less than 10% by molar ratio relative to all N-glycans) or undetectable amounts of Man₄ (GlcNAc)₂. In some embodiments, the compositions contain less than 80%, 75%, 70%, 65%, 60%, 55%, 50%, 45%, 40%, 35% 30%, 25%, 20%, 15%, 10%, 9%, 8%, 7%, 6%, 5%, 4%, 3%, 2%, or 1% (by molar ratio, relative to all N-glycans) or less complex-type glycans.

Glycan composition can be assessed using, e.g., lectin blotting, HPLC and/or mass spectrometry analysis, such as MALDI-TOF (See e.g., Townsend et al. (1997), Techniques in Glybiology, CRC Press.)

In some embodiments, the binding polypeptides carrying oligomannose-type glycans exhibit enhanced ADCC activity as compared to the same binding polypeptides produced without the mannosidase inhibitor (e.g., kifunensine) treatment. In other embodiments, the binding polypeptides carrying oligomannose-type glycans exhibited enhanced binding to an Fc receptor. In exemplary embodiments, the binding polypeptides carrying oligomannose-type glycans exhibited enhanced binding to an Fcγ receptor. In further exemplary embodiments, the binding polypeptides carrying oligomannose-type glycans exhibited enhanced binding to FcγRIIIa.

In still other embodiments, the binding polypeptides carrying oligomannose-type glycans exhibit substantially same or better binding specificity for the target. In some embodiments, the binding polypeptides carrying oligomannose-type glycans exhibit substantially the same or higher binding affinity for the target. In some embodiments, the binding polypeptides carrying oligomannose-type glycans exhibit substantially same or lower binding affinity for mannose receptor.

### Determining Binding Polypeptide Binding and Specificity

The binding affinity of an antibody or Fc fusion protein to its target as well as to Fc receptors and mannose receptors may be assessed using surface plasmon resonance, ELISA, or other suitable method (see Shields R L et al. (2001), J. Biol. Chem., vol. 276:6591-6604). In some embodiments, the binding constant K_{D} of a binding polypeptide for an Fc receptor may be above that of the wild-type control by 1, 2, 3, 4, 5, 6, 7, 8, 9, 10, 11, 12, 13, 14, 15, 16, 17, 18, 19, or 20-fold or higher. The binding constant K_{D} of a binding polypeptide for its target (e.g., antigen) may be substantially the same (i.e., ±50%) as the wild-type control or above it. In some embodiments, the binding constant K_{D} of an antibody or Fc fusion protein of the invention for mannose receptors may be substantially the same (i.e., ±50%) as the wild-type control or below it.

The binding specificity of an antibody or Fc fusion protein can be determined by, e.g., flow cytometry, Western blotting, or another suitable method. In some embodiments, a binding polypeptide is directed against a human target protein (e.g. a human antigen) expressed on the surface of a target cell. In some embodiments, it may be directed against a soluble antigen. In some other embodiments, a binding polypeptide is directed against a pathogenic target (e.g., viral or bacterial protein). The binding polypeptide may be either specific to a human target or may cross-react with corresponding targets from other species.

In some embodiments, certain pharmacokinetic parameters of a binding polypeptide of the invention are same or better that those of wild-type control. For example, in some embodiments, elimination half-life (t_{1/2}) and/or the area under the concentration curve (AUC) may be substantially the same (i.e., ±50%) as the wild-type control or above it. Pharmacokinetic parameters can be measured in humans or using an appropriate animal model. See, e.g., Shargel L and Yu A (1995), Applied Biopharmaceutics and Pharmacokinetics, 4th ed., McGraw-Hill/Appleton.

### Fc Domain and Fc Modifications

In certain aspects of the disclosure, Fc domains, e.g., Fc domain variants, are provided. As used herein, the term “Fc region” or “Fc domain” refers to the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc region comprises at least a hinge domain, a CH2 domain, and a CH3 domain.

The Fc region of an antibody is involved in non-antigen binding and can mediate effector function by binding to an Fc receptor. There are several different types of Fc receptors, which are classified based on the type of antibody that they recognize. For example, Fc-gamma receptors (FcγR) bind to IgG class antibodies, Fc-alpha receptors (FcαR) bind to IgA class antibodies, and Fc-epsilon receptors (FcεR) bind to IgE class antibodies. The neonatal Fc receptor (FcRn) interacts with the Fc region of an antibody to promote antibody recycling through rescue of normal lysosomal degradation. The FcγRs belong to a family that includes several members, e.g., FcγRI, FcγRIIa, FcγRIIb, FcγRIIIa, and FcγRIIIb.

The term “native Fc” or “wild-type Fc,” as used herein, refers to a molecule corresponding to the sequence of a non-antigen-binding fragment resulting from digestion of an antibody or produced by other means, whether in monomeric or multimeric form, and can contain the hinge region. The original immunoglobulin source of the native Fc is typically of human origin and can be any of the immunoglobulins, such as IgG1 and IgG2. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc,” as used herein, is generic to the monomeric, dimeric, and multimeric forms.

The term “Fc domain variant,” “Fc variant” or “modified Fc,” as used herein, refers to a molecule or sequence that is modified from a native/wild-type Fc but still comprises a binding site for an FcR. Thus, the term “Fc variant” can comprise a molecule or sequence that is humanized from a non-human native Fc. Furthermore, a native Fc comprises regions that can be removed because they provide structural features or biological activities that are not required for the antibody-like binding polypeptides described herein. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

In certain exemplary embodiments, an Fc variant featured herein has one or more of increased serum half-life, enhanced FcRn binding affinity, enhanced FcRn binding affinity at acidic pH, enhanced FcγRIIIa binding affinity, and/or similar thermal stability, as compared to a wild-type Fc.

FcγRIIIa V158, or human CD16a-V receptor, or CD16aV, refers to a polypeptide construct comprising a fragment of the CD16 human receptor binding to a Fc region of a natural antibody, mediating antibody-dependent cellular cytotoxicity and bearing a Valine (V) on position 158, which is also reported in the literature as allotype CD16a V158. FcγRIIIa F158, or human CD16a-F receptor, or CD16aF, refers to a polypeptide construct comprising a fragment of the CD16 human receptor binding to a Fc region of a natural antibody, mediating antibody-dependent cellular cytotoxicity and bearing a Phenylalanine (F) on position 158, which is also reported in the literature as allotype CD16a F158.

The term “Fc domain” as used herein encompasses native/wild-type Fc and Fc variants and sequences as defined herein. As with Fc variants and native Fc molecules, the term “Fc domain” includes molecules in monomeric or multimeric form, whether digested from whole antibody or produced by other means. In certain exemplary embodiments, an Fc domain as described herein is thermally stabilized.

In certain exemplary embodiments, an Fc domain as described herein is glycosylated (e.g., via N-linked glycosylation). In some embodiments, an Fc domain comprises N-linked glycosylation, e.g., at an N-linked glycosylation motif that contains the amino acid sequence NXT or NXS (X being any amino acid residue except proline). In some embodiments, an Fc domain is glycosylated at the native glycosylation site corresponding to amino acid position 297 of the Fc region, according to EU numbering.

In certain exemplary embodiments, the Fc domain is glycosylated with oligomannose-type N-glycans. In other exemplary embodiments, the glycosylated Fc domain contains oligomannose-type N-glycans selected from the group consisting of Man₉(GlcNAc)₂, Man₈(GlcNAc)₂, Man₇ (GlcNAc)₂, Man₆ (GlcNAc)₂, and Man₅ (GlcNAc)₂. In still other exemplary embodiments, the Fc domain contains oligomannose-type N-glycans that are predominately Man₉ (GlcNAc)₂ and Man₈ (GlcNAc)₂. In some exemplary embodiments, the Fc domain contains oligomannose-type N-glycans that are predominately Man₉ (GlcNAc)₂. In certain exemplary embodiments, the Fc domain is glycosylated with the oligomannose-type N-glycan at the native Fc glycosylation site corresponding to the EU position 297.

In other exemplary embodiment, the Fc domain is glycosylated with an oligomannose-type N-glycan at an engineered (non-native) Fc glycosylation site. Exemplary non-native Fc glycosylation sites comprise an asparagine residue at EU position 298, a serine or threonine residue at amino acid position 300, and optionally an alanine residue at EU position 299 and/or a glutamine residue at EU position 297. Exemplary non-native Fc glycosylation sites include the “NNAS” glycosylation motif described in U.S. Pat. No. 9,790,268 which is incorporated by reference herein.

In certain exemplary embodiments, an Fc domain as described herein is any combination of thermally stabilized, contains oligomannose-type N-glycans, and an Fc variant.

In one aspect, the present disclosure provides an Fc domain variant comprising effector-enhancing amino acid substitutions.

In one embodiment, an Fc domain variant with altered FcγRIIIa binding comprising one or more amino acid substitutions as disclosed herein. In one embodiment, an Fc domain variant with enhanced FcγRIIIa binding affinity having one or more amino acid substitutions as disclosed herein. In one embodiment, an Fc domain variant with enhanced FcγRIIIa binding affinity comprises two or more amino acid substitutions as disclosed herein. In one embodiment, an Fc domain variant with enhanced FcγRIIIa binding affinity comprises three or more amino acid substitutions as disclosed herein. In one embodiment, an Fc domain variant with enhanced FcγRIIIa binding affinity comprises four or more amino acid substitutions as disclosed herein.

In an exemplary embodiment, a binding polypeptide or Fc domain variant disclosed herein has an increased affinity for binding to human FcγRIIIa of at least 2, 3, 4, 5, 10, 20, 30, 40, 50, 60, 70, 80, 90, or 100-fold higher compared to a WT binding polypeptide.

In one embodiment, an Fc domain variant with altered FcRn binding comprises an Fc domain having one or more amino acid substitutions as disclosed herein. In one embodiment, an Fc domain variant with enhanced FcRn binding affinity comprises an Fc domain having one or more amino acid substitutions as disclosed herein. In one embodiment, an Fc domain variant with enhanced FcRn binding affinity comprises an Fc domain having two or more amino acid substitutions as disclosed herein. In one embodiment, an Fc domain variant with enhanced FcRn binding affinity comprises an Fc domain having three or more amino acid substitutions as disclosed herein.

In some embodiments, an Fc domain variant may exhibit a species-specific FcRn binding affinity. In one embodiment, an Fc domain variant may exhibit human FcRn binding affinity. In one embodiment, an Fc domain variant may exhibit cyno FcRn binding affinity. In some embodiments, an Fc domain variant may exhibit cross-species FcRn binding affinity. Such Fc domain variants are said to be cross-reactive across one or more different species. In one embodiment, an Fc domain variant may exhibit both human and cyno FcRn binding affinity.

The neonatal Fc receptor (FcRn) interacts with the Fc region of antibodies to promote recycling through rescue of normal lysosomal degradation. This process is a pH-dependent process that occurs in the endosomes at acidic pH (e.g., a pH less than 6.5) but not under the physiological pH conditions of the bloodstream (e.g., a non-acidic pH). In some embodiments, an Fc domain variant has enhanced FcRn binding affinity at an acidic pH compared to a wild-type Fc domain. In some embodiments, an Fc domain variant has enhanced FcRn binding affinity at pH less than 7, e.g., at about pH 6.5, at about pH 6.0, at about pH 5.5, at about pH 5.0, compared to a wild-type Fc domain. In some embodiments, an Fc domain variant has enhanced FcRn binding affinity at pH less than 7, e.g., at about pH 6.5, at about pH 6.0, at about pH 5.5, at about pH 5.0, compared to the FcRn binding affinity of a wild-type Fc domain at an elevated non-acidic pH. An elevated non-acidic pH can be, e.g., pH greater than 7, about pH 7, about pH 7.4, about pH 7.6, about pH 7.8, about pH 8.0, about pH 8.5, about pH 9.0.

In certain embodiments, it may be desired for an Fc domain variant to exhibit approximately the same FcRn binding affinity at non-acidic pH as a wild-type Fc domain. In some embodiments, it may be desired for an Fc domain variant to exhibit less FcRn binding affinity at non-acidic pH than a binding polypeptide comprising a modified Fc domain having the double amino acid substitution M428L/N434S, according to EU numbering. See U.S. Pat. No. 8,088,376. Accordingly, it may be desired an Fc domain variant to exhibit minimal perturbation to pH-dependent FcRn binding.

In some embodiments, an Fc domain variant having enhanced FcRn binding affinity at an acidic pH, has a reduced (i.e., slower) FcRn off-rate as compared to a wild-type Fc domain. In some embodiments, an Fc domain variant having enhanced FcRn binding affinity at an acidic pH compared to the FcRn binding affinity of the binding polypeptide at an elevated non-acidic pH, has a slower FcRn off-rate at the acidic pH compared to the FcRn off-rate of a wild-type Fc domain at the elevated non-acidic pH.

Certain embodiments include Fc domain variants in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, or increased serum half-life when compared with a whole, unaltered antibody of approximately the same immunogenicity.

In certain other embodiments, an Fc domain variant comprises constant regions derived from different antibody isotypes (e.g., constant regions from two or more of a human IgG1, IgG2, IgG3, or IgG4). In other embodiments, an Fc domain variant comprises a chimeric hinge (i.e., a hinge comprising hinge portions derived from hinge domains of different antibody isotypes, e.g., an upper hinge domain from an IgG4 molecule and an IgG1 middle hinge domain). In certain embodiments, the Fc domain may be mutated to increase or decrease effector function using techniques known in the art.

In some embodiments, an Fc domain variant has altered binding affinity to an Fc receptor. There are several different types of Fc receptors, which are classified based on the type of antibody that they recognize. For example, Fc-gamma receptors (FcγR) bind to IgG class antibodies, Fc-alpha receptors (FcαR) bind to IgA class antibodies, and Fc-epsilon receptors (FcεR) bind to IgE class antibodies. The FcγRs belong to a family that includes several members, e.g., FcγRI, FcγRIIa, FcγRIIb, FcγRIIIa, and FcγRIIIb. In some embodiments, an Fc domain variant has altered FcγRIIIa binding affinity, compared to a wild-type Fc domain. In some embodiments, an Fc domain variant has reduced FcγRIIIa binding affinity, compared to a wild-type Fc domain. In some embodiments, an Fc domain variant has enhanced FcγRIIIa binding affinity, compared to a wild-type Fc domain. In some embodiments, an Fc domain variant modified Fc domain has approximately the same FcγRIIIa binding affinity, compared to a wild-type Fc domain.

In some embodiments, an Fc domain variant has altered binding affinity to an Fc receptor (e.g., an increased affinity to an FcγRIIIa receptor) and thermal stability similar to a binding polypeptide with a wild-type Fc domain. In certain embodiments, the Fc variant has a Tm within 1, 2, 3, 4, 5, 6, 7, 8, 9, or 10 degrees Celsius of a binding polypeptide with a wild-type Fc domain. In an exemplary embodiment, the Fc variant has a melting temperature (Tm) within 10 degrees Celsius of a binding polypeptide with a wild-type Fc domain.

In some embodiments, an Fc domain variant has altered binding affinity to an Fc receptor (e.g., an increased affinity to an FcγRIIIa receptor) and thermal stability similar to a binding polypeptide with a wild-type Fc domain, wherein the binding polypeptide with the variant Fc domain is produced by culturing a cell that expresses the binding polypeptide in the presence of kifunensine and the binding polypeptide with the wild-type Fc domain is produced by culturing a cell in the absence of kifunensine. In certain embodiments, the Fc variant has a Tm within 1, 2, 3, 4, 5, 6, 7, 8, 9, or 10 degrees Celsius of a binding polypeptide with a wild-type Fc domain, wherein the binding polypeptide with the variant Fc domain is produced by culturing a cell that expresses the binding polypeptide in the presence of kifunensine and the binding polypeptide with the wild-type Fc domain is produced by culturing a cell in the absence of kifunensine. In an exemplary embodiment, the Fc variant has a Tm within 10 degrees Celsius of a binding polypeptide with a wild-type Fc domain, wherein the binding polypeptide with the variant Fc domain is produced by culturing a cell that expresses the binding polypeptide in the presence of kifunensine and the binding polypeptide with the wild-type Fc domain is produced by culturing a cell in the absence of kifunensine.

In some embodiments, an Fc domain variant has altered binding affinity to an Fc receptor (e.g., an increased affinity to an FcγRIIIa receptor) and thermal stability similar to a binding polypeptide with a wild-type Fc domain, wherein both the binding polypeptide with the variant Fc domain and the binding polypeptide with the wild-type Fc domain are produced by culturing a cell that expresses the binding polypeptide in the presence of kifunensine. In certain embodiments, the Fc variant has a Tm within 1, 2, 3, 4, or 5 degrees Celsius of a binding polypeptide with a wild-type Fc domain, wherein both the binding polypeptide with the variant Fc domain and the binding polypeptide with the wild-type Fc domain are produced by culturing a cell that expresses the binding polypeptide in the presence of kifunensine. In an exemplary embodiment, the Fc variant has a Tm within 5 degrees Celsius of a binding polypeptide with a wild-type Fc domain, wherein both the binding polypeptide with the variant Fc domain and the binding polypeptide with the wild-type Fc domain are produced by culturing a cell that expresses the binding polypeptide in the presence of kifunensine.

In certain embodiments, binding polypeptides may comprise an antibody constant region (e.g., an IgG constant region e.g., a human IgG constant region, e.g., a human IgG1 or IgG4 constant region) which mediates one or more effector functions. For example, binding of the C1-complex to an antibody constant region may activate the complement system. Activation of the complement system is important in the opsonisation and lysis of cell pathogens. The activation of the complement system also stimulates the inflammatory response and may also be involved in autoimmune hypersensitivity. Further, antibodies bind to receptors on various cells via the Fc region (Fc receptor binding sites on the antibody Fc region bind to Fc receptors (FcRs) on a cell). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In some embodiments, the binding polypeptides (e.g., antibodies or antigen binding fragments thereof) bind to an Fc-gamma receptor. In alternative embodiments, binding polypeptides may comprise a constant region which is devoid of one or more effector functions (e.g., ADCC activity) and/or is unable to bind Fcγ receptor.

Disclosed herein are binding polypeptides with enhanced ADCC activity. “ADCC activity” as used herein refers to the ability of a binding polypeptide to elicit an ADCC reaction. ADCC is a cell-mediated reaction in which antigen-nonspecific cytotoxic cells that express FcRs (e.g., natural killer (NK) cells, neutrophils, and macrophages) recognize binding polypeptide bound to the surface of a target cell and subsequently cause lysis of (i.e., “kill”) the target cell. The primary mediator cells are natural killer (NK) cells. NK cells express FcγRIII only, with FcγRIIIa being an activating receptor and FcγRIIIb an inhibiting one; monocytes express FcγRI, FcγRII and FcγRIII (Ravetch et al. (1991), Annu. Rev. Immunol., vol. 9:457-92). ADCC activity can be assessed directly using an in vitro assay, e.g., a release assay using peripheral blood mononuclear cells (PBMC) and/or NK effector cells, or a bioluminescent reporter bioassay as described in the examples (see also Shields R L et al. (2001), J. Biol. Chem., vol. 276 (9): 6591-6604). ADCC activity may be expressed as a concentration of binding polypeptide at which the lysis of target cells is half-maximal. Accordingly, in some embodiments, the concentration of binding polypeptide of the invention, at which the lysis level is the same as the half-maximal lysis level by the wild-type control, is at least 2, 3, 5, 6, 7, 8, 9, 10, 20, 30, 40, 50, 60, 70, 80, 90, or 100-fold lower than the concentration of the wild-type control itself. Additionally, in some embodiments, the binding polypeptide of the invention may exhibit a higher maximal target cell lysis as compared to the wild-type control. For example, the maximal target cell lysis of an antibody or Fc fusion protein of the invention may be 10%, 11%, 12%, 13%, 14%, 15%, 16%, 17%, 18%, 19%, 20%, 21%, 22%, 23%, 24%, 25% or higher than that of the wild-type control.

In an exemplary embodiment, a binding polypeptide or Fc domain variant disclosed herein has increased antibody-dependent cellular cytotoxicity (ADCC) activity compared to a WT binding polypeptide. In a further exemplary embodiment, the ADCC activity of the binding polypeptide or Fc domain variant is at least 1, 2, 3, 4, or 5-fold higher compared to the WT binding polypeptide.

Certain embodiments include antibodies in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain other embodiments, binding polypeptides comprise constant regions derived from different antibody isotypes (e.g., constant regions from two or more of a human IgG1, IgG2, IgG3, or IgG4). In other embodiments, binding polypeptides comprises a chimeric hinge (i.e., a hinge comprising hinge portions derived from hinge domains of different antibody isotypes, e.g., an upper hinge domain from an IgG4 molecule and an IgG1 middle hinge domain). In one embodiment, binding polypeptides comprise an Fc region or portion thereof from a human IgG4 molecule and a Ser228Pro mutation (EU numbering) in the core hinge region of the molecule.

The amino acid substitution(s) of an Fc variant may be located at any position (i.e., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The binding polypeptides may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated in its entirety by reference herein. In one exemplary embodiment, a binding polypeptide may comprise an Fc variant comprising an amino acid substitution at EU position 268 (e.g., H268D or H268E). In another exemplary embodiment, a binding polypeptide may comprise an amino acid substitution at EU position 239 (e.g., S239D or S239E) and/or EU position 332 (e.g., I332D or I332Q).

In certain embodiments, a binding polypeptide may comprise an Fc variant comprising an amino acid substitution which alters the antigen-independent effector functions of the antibody, in particular the circulating half-life of the binding polypeptide. Such binding polypeptides exhibit either increased or decreased binding to FcRn when compared to binding polypeptides lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include applications localized to the brain, kidney, and/or liver. In one exemplary embodiment, the altered binding polypeptides (e.g., antibodies or antigen binding fragments thereof) exhibit reduced transport across the epithelium of kidney glomeruli from the vasculature. In another embodiment, the altered binding polypeptides (e.g., antibodies or antigen binding fragments thereof) exhibit reduced transport across the blood brain barrier (BBB) from the brain into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions which alter FcRn binding activity are disclosed in International PCT Publication No. WO05/047327 which is incorporated in its entirety by reference herein. In certain exemplary embodiments, the binding polypeptides (e.g., antibodies or antigen binding fragments thereof) comprise an Fc domain having one or more of the following substitutions: V284E, H285E, N286D, K290E and S304D (EU numbering). In yet other exemplary embodiments, the binding molecules comprise a human Fc domain with the double mutation H433K/N434F (see, e.g., U.S. Pat. No. 8,163,881).

In other embodiments, binding polypeptides, for use in the diagnostic and treatment methods described herein have a constant region, e.g., an IgG1 or IgG4 heavy chain constant region, which is altered to reduce or eliminate glycosylation. For example, binding polypeptides (e.g., antibodies or antigen binding fragments thereof) may also comprise an Fc variant comprising an amino acid substitution which alters the glycosylation of the antibody Fc. For example, said Fc variant may have reduced glycosylation (e.g., N- or O-linked glycosylation). In exemplary embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In a particular embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are disclosed in International PCT Publication No. WO05/018572, which is incorporated in its entirety by reference herein. In some embodiments, the binding polypeptides are modified to eliminate glycosylation. Such binding polypeptides may be referred to as “agly” binding polypeptides (e.g., “agly” antibodies). While not being bound by theory, it is believed that “agly” binding polypeptides may have an improved safety and stability profile in vivo. Agly binding polypeptides can be of any isotype or subclass thereof, e.g., IgG1, IgG2, IgG3, or IgG4. In certain embodiments, agly binding polypeptides comprise an aglycosylated Fc region of an IgG4 antibody which is devoid of Fc-effector function, thereby eliminating the potential for Fc mediated toxicity to the normal vital organs that express IL-6. In yet other embodiments, binding polypeptides comprise an altered glycan. For example, the antibody may have a reduced number of fucose residues on an N-glycan at Asn297 of the Fc region, i.e., is afucosylated. Afucosylation increases FcγRII binding on the NK cells and potently increases ADCC. It has been shown that a diabody comprising an anti-IL-6 scFv and an anti-CD3 scFv induces killing of IL-6 expressing cells by ADCC. Accordingly, in one embodiment, an afucosylated anti-IL-6 antibody is used to target and kill IL-6-expressing cells. In another embodiment, the binding polypeptide may have an altered number of sialic acid residues on the N-glycan at Asn297 of the Fc region. Numerous art-recognized methods are available for making “agly” antibodies or antibodies with altered glycans. For example, genetically engineered host cells (e.g., modified yeast, e.g., Picchia, or CHO cells) with modified glycosylation pathways (e.g., glycosyl-transferase deletions) can be used to produce such antibodies.

In certain exemplary embodiments, an effector-enhancing Fc domain variant has one or more amino acid substitutions selected from the group consisting of: an aspartic acid (D) at amino acid position 221; a cysteine (C) at amino acid position 222; a tyrosine (Y) at amino acid position 234; an alanine (A) at amino acid position 236; a tryptophan (W) at amino acid position 236; an aspartic acid (D) at amino acid position 239; a leucine (L) at amino acid position 243; a tyrosine (Y) at amino acid position 252; a threonine (T) at amino acid position 254; an aspartic acid (D) at amino acid position 256; a glutamic acid (E) at amino acid position 256; a glutamic acid (E) at amino acid position 267; a phenylalanine (F) at amino acid position 268; a proline (P) at amino acid position 292; an alanine (A) at amino acid position 298; a leucine (L) at amino acid position 300; an isoleucine (I) at amino acid position 305; a tryptophan (W) at amino acid position 307; a glutamine (Q) at amino acid position 307; a threonine (T) at amino acid position 324; a tryptophan (W) at amino acid position 326; an alanine (A) at amino acid position 326; a leucine (L) at amino acid position 330; a glutamic acid (E) at amino acid position 332; an alanine (A) at amino acid position 333; a serine(S) at amino acid position 333; an alanine (A) at amino acid position 334; an alanine (A) at amino acid position 336; an arginine (R) at amino acid position 345; a leucine (L) at amino acid position 396; a leucine (L) at amino acid position 428; and a serine(S) at amino acid position 434, according to EU numbering. See Saunders K O (2009), Front. Immunol., vol. 10 (1296): 1-20; Mackness et al. (2019), MAbs, vol. 11:1276-88; and WO2019147973A1.

In some embodiments, an Fc domain variant may comprise an amino acid substitution at positions selected from amino acid positions 239, 267, 268, 298, 314, 330, 332, 339, and 373 according to EU numbering. In some embodiments, an Fc domain variant may comprise an aspartic acid (D) at amino acid position 239. In other embodiments, an Fc domain variant may comprise a glutamic acid (E) at amino acid position 332. In still other embodiments, an Fc domain variant may comprise an alanine (A) at amino acid position 298. In an exemplary embodiment, an Fc domain variant comprises an aspartic acid (D) at amino acid position 239 and a glutamic acid (E) at amino acid position 332. In an exemplary embodiment, an Fc domain variant comprises an aspartic acid (D) at amino acid position 239 and an alanine (A) at amino acid position 298.

In some exemplary embodiments, an Fc domain variant may comprise an aspartic acid (D) at amino acid position 267. In other embodiments, an Fc domain variant may comprise an aspartic acid (D) at amino acid position 268. In still other embodiments, an Fc domain variant may comprise a glutamic acid (E) at amino acid position 268. In some embodiments, an Fc domain variant may comprise a cysteine (C) at amino acid position 298. In an exemplary embodiment, an Fc domain variant comprises an isoleucine (I) at amino acid position 314. In some embodiments, an Fc domain variant may comprise a methionine (M) at amino acid position 314. In other embodiments, an Fc domain variant may comprise a glutamine (Q) at amino acid position 314. In still other embodiments, an Fc domain variant may comprise a tryptophan (W) at amino acid position 314. In an exemplary embodiment, an Fc domain variant comprises a phenylalanine (F) at amino acid position 330. In another exemplary embodiment, an Fc domain variant comprises a methionine (M) at amino acid position 330. In yet another exemplary embodiment, an Fc domain variant comprises an aspartic acid (D) at amino acid position 339. In yet another exemplary embodiment, an Fc domain variant comprises an isoleucine (I) at amino acid position 339. In yet another exemplary embodiment, an Fc domain variant comprises a proline (P) at amino acid position 339. In yet another exemplary embodiment, an Fc domain variant comprises a threonine (T) at amino acid position 339. In another exemplary embodiment, an Fc domain variant comprises a phenylalanine (F) at amino acid position 373. In yet another exemplary embodiment, an Fc domain variant comprises a tryptophan (W) at amino acid position 373.

In some embodiments, an Fc domain variant may comprise an amino acid substitution at positions selected from amino acid positions 252, 254, 256, 307, 428, and 434 according to EU numbering. In some embodiments, an Fc domain variant may comprise a leucine (L) at amino acid position 428; and a serine(S) at amino acid position 434. In other embodiments, an Fc domain variant may comprise a tyrosine (Y) at amino acid position 252, an aspartic acid (D) at amino acid position 256. In still other embodiments, an Fc domain variant may comprise an aspartic acid (D) at amino acid position 256, a tryptophan (W) at amino acid position 307. In some embodiments, an Fc domain variant may comprise a tyrosine (Y) at amino acid position 252, a threonine (T) at amino acid position 254, and a glutamic acid (E) at amino acid position 256.

In some embodiments, an Fc domain variant may further comprise an amino acid substitution at amino acid positions 256 and/or 307, according to EU numbering. In some embodiments, an Fc domain variant may comprise the combination of amino acid substitutions comprising an aspartic acid (D) at amino acid positions 256 and a glutamine (Q) at amino acid position 307. In an exemplary embodiment, an Fc domain variant comprises an aspartic acid (D) at amino acid positions 256, a glutamine (Q) at amino acid position 307, and an aspartic acid (D) at amino acid position 239. In another exemplary embodiment, an Fc domain variant comprises an aspartic acid (D) at amino acid positions 256, a glutamine (Q) at amino acid position 307, and an alanine (A) at amino acid position 298. In another exemplary embodiment, an Fc domain variant comprises an aspartic acid (D) at amino acid positions 256, a glutamine (Q) at amino acid position 307, and a glutamic acid (E) at amino acid position 332. In another exemplary embodiment, an Fc domain variant comprises an aspartic acid (D) at amino acid positions 256, a glutamine (Q) at amino acid position 307, an aspartic acid (D) at amino acid position 239, and a glutamic acid (E) at amino acid position 332. In still another exemplary embodiment, an Fc domain variant comprises an aspartic acid (D) at amino acid positions 256, a glutamine (Q) at amino acid position 307, an aspartic acid (D) at amino acid position 239 and an alanine (A) at amino acid position 298.

In one exemplary embodiment, an Fc domain variant comprises an aspartic acid (D) at amino acid positions 256, a glutamine (Q) at amino acid position 307, and an aspartic acid (D) at amino acid position 267. In another exemplary embodiment, an Fc domain variant comprises an aspartic acid (D) at amino acid positions 256, a glutamine (Q) at amino acid position 307, and an aspartic acid (D) at amino acid position 268. In still another exemplary embodiment, an Fc domain variant comprises an aspartic acid (D) at amino acid positions 256, a glutamine (Q) at amino acid position 307, and a glutamic acid (E) at amino acid position 268. In an exemplary embodiment, an Fc domain variant comprises an aspartic acid (D) at amino acid position 256, a glutamine (Q) at amino acid position 307, and a cysteine (C) at amino acid position 298. In an exemplary embodiment, an Fc domain variant comprises an aspartic acid (D) at amino acid positions 256, a glutamine (Q) at amino acid position 307, and an isoleucine (I) at amino acid position 314. In another exemplary embodiment, an Fc domain variant comprises an aspartic acid (D) at amino acid positions 256, a glutamine (Q) at amino acid position 307, and a methionine (M) at amino acid position 314. In an exemplary embodiment, an Fc domain variant comprises an aspartic acid (D) at amino acid positions 256, a glutamine (Q) at amino acid position 307, and a glutamine (Q) at amino acid position 314. In another exemplary embodiment, an Fc domain variant comprises an aspartic acid (D) at amino acid positions 256, a glutamine (Q) at amino acid position 307, and a tryptophan (W) at amino acid position 314. In still another exemplary embodiment, an Fc domain variant comprises an aspartic acid (D) at amino acid positions 256, a glutamine (Q) at amino acid position 307, and a phenylalanine (F) at amino acid position 330. In still another exemplary embodiment, an Fc domain variant comprises an aspartic acid (D) at amino acid positions 256, a glutamine (Q) at amino acid position 307, and a methionine (M) at amino acid position 330. In another exemplary embodiment, an Fc domain variant comprises an aspartic acid (D) at amino acid positions 256, a glutamine (Q) at amino acid position 307, and an aspartic acid (D) at amino acid position 339. In another exemplary embodiment, an Fc domain variant comprises an aspartic acid (D) at amino acid positions 256, a glutamine (Q) at amino acid position 307, and an isoleucine (I) at amino acid position 339. In another exemplary embodiment, an Fc domain variant comprises an aspartic acid (D) at amino acid positions 256, a glutamine (Q) at amino acid position 307, and a proline (P) at amino acid position 339. In another exemplary embodiment, an Fc domain variant comprises an aspartic acid (D) at amino acid positions 256, a glutamine (Q) at amino acid position 307, and a threonine (T) at amino acid position 339. In still another exemplary embodiment, an Fc domain variant comprises an aspartic acid (D) at amino acid positions 256, a glutamine (Q) at amino acid position 307, and a phenylalanine (F) at amino acid position 373. In another exemplary embodiment, an Fc domain variant comprises an aspartic acid (D) at amino acid positions 256, a glutamine (Q) at amino acid position 307, and a tryptophan (W) at amino acid position 373.

### Thermally-Stabilized Fc Domain Variants

The structure of constant antibody domains is similar to that of the variable domains consisting of β-strands connected with loops and short helices. The CH2 domain of the heavy constant regions exhibits weak carbohydrate-mediated interchain protein-protein interactions in contrast to the extensive interchain interactions exhibited in other domains. Isolated murine CH2 domains are relatively unstable at physiological temperature (Feige M J et al. (2004), J. Mol. Biol., vol. 344 (1): 107-118), but previous efforts demonstrate that the thermostability of CH2 domains may be enhanced with the addition of intrachain disulfide bonds, and that these could be used as scaffolds for binders (Gong R et al. (2009), J. Biol. Chem. vol. 284 (21): 14203-210).

Effector-enhancing Fc domain variants that exhibit increased thermal instability (i.e., decreased thermal stability) relative to a wild-type Fc domain are known. For example, S239D/I332E and S239D/I332E/A330L variants lead to decreased stability of the CH2 domain as indicated by the lowering of melting temperature (Tm) in differential scanning calorimetry (DSC) analysis. G236A/S239D/A330L/I332E has a reduced protein thermal shift measurement when compared to wild-type, as well as a considerably reduced half-life in hFcγR transgenic mice. See Liu Z et al. (2014), J. Biol. Chem., vol. 289 (6): 3571-90 and Liu R et al. (2020), Antibodies, vol. 9 (4): 64 for a review.

Effector-enhancing Fc domain variants having improved FcγR binding wherein stability is not significantly reduced as compared to wild-type are known. See, e.g., EP2940135B1 at Example 10.

It has been further discovered that thermally-stabilized Fc domain variants may be produced by introducing one or more disulfide bonds in the Fc domain. Accordingly, in one aspect, the present disclosure provides an Fc domain variant comprising one or more engineered (e.g., non-native) disulfide bonds, e.g., intrachain disulfide bonds mediated, e.g., by one or more pairs of cysteines.

In certain exemplary embodiments, a disulfide bond is an intrachain disulfide bond between the two CH2 regions of an Fc domain. In certain exemplary embodiments, a disulfide bond is an intrachain disulfide bond between the two CH3 regions of an Fc domain. In certain exemplary embodiments, two or more intrachain disulfide bonds are present in between the two CH2 regions of an Fc domain and/or between the two CH2 regions of an Fc domain.

Thermal stability, or the propensity of an Fc domain (e.g., an Fc domain with or without a binding polypeptide) to unfold, may be determined using a variety of methods known in the art. For example, the unfolding or denaturation temperature can be measured by nano-format differential scanning calorimetry (nanoDSC) or nano-format differential scanning fluorimetry (nanoDSF) (Wen J et al. (2020), Anal. Biochem., vol. 593:113581). The detectable temperature at which a protein begins to unfold is the Tonset.

In certain exemplary embodiments, the Tonset of a thermally-stabilized Fc domain variant (e.g., having one or more engineered disulfide bonds) is increased relative to an Fc domain variant that is not thermally stabilized. In certain exemplary embodiments, the Tonset of a thermally-stabilized Fc domain variant is increased by about 1.0° C., about 1.5° C., about 2.0° C., about 2.5° C., about 3.0° C., about 3.5° C., about 4.0° C., about 4.5° C., about 5.0° C., about 5.5° C., about 6.0° C., about 6.5° C., about 7.0° C., about 7.5° C., about 8.0° C., about 8.5° C., about 9.0° C., about 9.5° C., about 10.0° C., about 10.5° C., about 11.0° C., about 11.5° C., about 12.0° C., about 12.5° C., about 13.0° C., about 13.5° C., about 14.0° C., about 14.5° C., about 15.0° C., about 15.5° C., about 16.0° C., about 16.5° C., about 17.0° C., about 17.5° C., about 18.0° C., about 18.5° C., about 19.0° C., about 19.5° C., about 20.0° C., about 20.5° C., about 21.0° C., about 21.5° C., about 22.0° C., about 22.5° C., about 23.0° C., about 23.5° C., about 24.0° C., about 24.5° C. or about 25.0° C. relative to an Fc domain variant that is not thermally stabilized.

In certain exemplary embodiments, a thermally-stabilized Fc domain variant has one or more amino acid substitution pairs selected from the group consisting of cysteine substitutions at: amino acid positions 242 and 334; amino acid positions 240 and 334; amino acid positions 287 and 306; amino acid positions 292 and 302; amino acid positions 323 and 332; amino acid positions 259 and 306; amino acid positions 350 and 441; amino acid positions 343 and 431; amino acid positions 375 and 404; amino acid positions 375 and 396; and amino acid positions 348 and 439, according to EU numbering. See Wozniak-Knopp G et al. (2012), PLoS One, vol. 7 (1): e30083, Jacobsen F W et al. (2017), J. Biol. Chem. 292:1865-75, and WO2014153063 for a review.

In certain exemplary embodiments, a thermally-stabilized Fc domain variant comprises an engineered (e.g., a non-native) intrachain disulfide bond mediated by a pair of cysteines that substitute for (i) a leucine (L) at amino acid position 242 and a lysine (K) at amino acid position 334; (ii) an alanine (A) at amino acid position 287 and a leucine (L) at amino acid position 306; or (iii) an arginine (R) at amino acid position 292 and a valine (V) at amino acid position 302, according to EU numbering.

In some embodiments, a thermally-stabilized Fc domain variant comprises an engineered (e.g., a non-native) intrachain disulfide bond mediated by a pair of cysteines that substitute for a leucine (L) at amino acid position 242 and a lysine (K) at amino acid position 334. In certain embodiments, a thermally-stabilized Fc domain variant comprises an engineered (e.g., a non-native) intrachain disulfide bond mediated by a pair of cysteines that substitute an alanine (A) at amino acid position 287 and a leucine (L) at amino acid position 306. In certain exemplary embodiments, a thermally-stabilized Fc domain variant comprises an engineered (e.g., a non-native) intrachain disulfide bond mediated by a pair of cysteines that substitute for an arginine (R) at amino acid position 292 and a valine (V) at amino acid position 302. In certain exemplary embodiments, a thermally-stabilized Fc domain variant may comprise at least one engineered intrachain disulfide bond. In certain embodiments, a thermally-stabilized Fc domain variant may comprise more than one engineered intrachain disulfide bond.

In an exemplary embodiment, an Fc domain variant comprises an aspartic acid (D) at amino acid position 239, a cysteine (C) at amino acid position 292, and a cysteine (C) at amino acid position 302. In other another exemplary embodiment, an Fc domain variant may comprise a glutamic acid (E) at amino acid position 332, a cysteine (C) at amino acid position 292, and a cysteine (C) at amino acid position 302. In still another exemplary embodiment, an Fc domain variant comprises an alanine (A) at amino acid position 298, a cysteine (C) at amino acid position 292, and a cysteine (C) at amino acid position 302. In a further exemplary embodiment, an Fc domain variant comprises an aspartic acid (D) at amino acid position 239, a glutamic acid (E) at amino acid position 332, a cysteine (C) at amino acid position 292, and a cysteine (C) at amino acid position 302. In another exemplary embodiment, an Fc domain variant comprises an aspartic acid (D) at amino acid position 239, an alanine (A) at amino acid position 298, and a cysteine (C) at amino acid position 292, and a cysteine (C) at amino acid position 302.

In an exemplary embodiment, an Fc domain variant comprises an aspartic acid (D) at amino acid position 267, a cysteine (C) at amino acid position 292, and a cysteine (C) at amino acid position 302. In another exemplary embodiment, an Fc domain variant comprises an aspartic acid (D) at amino acid position 268, a cysteine (C) at amino acid position 292, and a cysteine (C) at amino acid position 302. In still another exemplary embodiment, an Fc domain variant comprises a glutamic acid (E) at amino acid position 268, a cysteine (C) at amino acid position 292, and a cysteine (C) at amino acid position 302. In another exemplary embodiment, an Fc domain variant comprises a cysteine (C) at amino acid position 298, a cysteine (C) at amino acid position 292, and a cysteine (C) at amino acid position 302. In an exemplary embodiment, an Fc domain variant comprises an isoleucine (I) at amino acid position 314, a cysteine (C) at amino acid position 292, and a cysteine (C) at amino acid position 302. In another exemplary embodiment, an Fc domain variant comprises a methionine (M) at amino acid position 314, a cysteine (C) at amino acid position 292, and a cysteine (C) at amino acid position 302. In an exemplary embodiment, an Fc domain variant comprises a glutamine (Q) at amino acid position 314, a cysteine (C) at amino acid position 292, and a cysteine (C) at amino acid position 302. In another exemplary embodiment, an Fc domain variant comprises a tryptophan (W) at amino acid position 314, a cysteine (C) at amino acid position 292, and a cysteine (C) at amino acid position 302. In still another exemplary embodiment, an Fc domain variant comprises a phenylalanine (F) at amino acid position 330, a cysteine (C) at amino acid position 292, and a cysteine (C) at amino acid position 302. In yet another exemplary embodiment, an Fc domain variant comprises a methionine (M) at amino acid position 330, a cysteine (C) at amino acid position 292, and a cysteine (C) at amino acid position 302. In another exemplary embodiment, an Fc domain variant comprises an aspartic acid (D) at amino acid position 339, a cysteine (C) at amino acid position 292, and a cysteine (C) at amino acid position 302. In another exemplary embodiment, an Fc domain variant comprises an isoleucine (I) at amino acid position 339, a cysteine (C) at amino acid position 292, and a cysteine (C) at amino acid position 302. In another exemplary embodiment, an Fc domain variant comprises a proline (P) at amino acid position 339, a cysteine (C) at amino acid position 292, and a cysteine (C) at amino acid position 302. In another exemplary embodiment, an Fc domain variant comprises a threonine (T) at amino acid position 339, a cysteine (C) at amino acid position 292, and a cysteine (C) at amino acid position 302. In another exemplary embodiment, an Fc domain variant comprises a phenylalanine (F) at amino acid position 373, a cysteine (C) at amino acid position 292, and a cysteine (C) at amino acid position 302. In another exemplary embodiment, an Fc domain variant comprises a tryptophan (W) at amino acid position 373, a cysteine (C) at amino acid position 292, and a cysteine (C) at amino acid position 302.

In other exemplary embodiments, an Fc domain variant comprises a cysteine (C) at amino acid position 292, and a cysteine (C) at amino acid position 302, an aspartic acid (D) at amino acid position 256, and a glutamine (Q) at amino acid position 307.

In another exemplary embodiment, an Fc domain variant comprises an aspartic acid (D) at amino acid position 239, a cysteine (C) at amino acid position 292, and a cysteine (C) at amino acid position 302, an aspartic acid (D) at amino acid position 256, and a glutamine (Q) at amino acid position 307. In yet another exemplary embodiment, an Fc domain variant comprises an alanine (A) at amino acid position 298, a cysteine (C) at amino acid position 292, and a cysteine (C) at amino acid position 302, an aspartic acid (D) at amino acid position 256, and a glutamine (Q) at amino acid position 307. In another exemplary embodiment, an Fc domain variant comprises an aspartic acid (D) at amino acid position 239, an alanine (A) at amino acid position 298, a cysteine (C) at amino acid position 292, and a cysteine (C) at amino acid position 302, an aspartic acid (D) at amino acid position 256, and a glutamine (Q) at amino acid position 307. In yet another exemplary embodiment, an Fc domain variant comprises a glutamic acid (E) at amino acid position 332, a cysteine (C) at amino acid position 292, and a cysteine (C) at amino acid position 302, an aspartic acid (D) at amino acid position 256, and a glutamine (Q) at amino acid position 307. In yet another exemplary embodiment, an Fc domain variant comprises an aspartic acid (D) at amino acid position 239, a glutamic acid (E) at amino acid position 332, a cysteine (C) at amino acid position 292, and a cysteine (C) at amino acid position 302, an aspartic acid (D) at amino acid position 256, and a glutamine (Q) at amino acid position 307. In an exemplary embodiment, an Fc domain variant comprises an aspartic acid (D) at amino acid position 267, a cysteine (C) at amino acid position 292, a cysteine (C) at amino acid position 302, an aspartic acid (D) at amino acid position 256, and a glutamine (Q) at amino acid position 307. In an exemplary embodiment, an Fc domain variant comprises a cysteine (C) at amino acid position 298, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises an aspartic acid (D) at amino acid position 268, a cysteine (C) at amino acid position 292, a cysteine (C) at amino acid position 302, an aspartic acid (D) at amino acid position 256, and a glutamine (Q) at amino acid position 307. In still another exemplary embodiment, an Fc domain variant comprises a glutamic acid (E) at amino acid position 268, a cysteine (C) at amino acid position 292, a cysteine (C) at amino acid position 302, an aspartic acid (D) at amino acid position 256, and a glutamine (Q) at amino acid position 307. In an exemplary embodiment, an Fc domain variant comprises an isoleucine (I) at amino acid position 314, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises a methionine (M) at amino acid position 314, a cysteine (C) at amino acid position 292, a cysteine (C) at amino acid position 302, an aspartic acid (D) at amino acid position 256, and a glutamine (Q) at amino acid position 307. In an exemplary embodiment, an Fc domain variant comprises a glutamine (Q) at amino acid position 314, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises a tryptophan (W) at amino acid position 314, a cysteine (C) at amino acid position 292, a cysteine (C) at amino acid position 302, an aspartic acid (D) at amino acid position 256, and a glutamine (Q) at amino acid position 307. In still another exemplary embodiment, an Fc domain variant comprises a phenylalanine (F) at amino acid position 330, a cysteine (C) at amino acid position 292, a cysteine (C) at amino acid position 302, an aspartic acid (D) at amino acid position 256, and a glutamine (Q) at amino acid position 307. In yet another exemplary embodiment, an Fc domain variant comprises a methionine (M) at amino acid position 330, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises an aspartic acid (D) at amino acid position 339, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises an isoleucine (I) at amino acid position 339, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises a proline (P) at amino acid position 339, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises a threonine (T) at amino acid position 339, a cysteine (C) at amino acid position 292, a cysteine (C) at amino acid position 302, an aspartic acid (D) at amino acid position 256, and a glutamine (Q) at amino acid position 307. In still another exemplary embodiment, an Fc domain variant comprises a phenylalanine (F) at amino acid position 373, a cysteine (C) at amino acid position 292, a cysteine (C) at amino acid position 302, an aspartic acid (D) at amino acid position 256, and a glutamine (Q) at amino acid position 307. In another exemplary embodiment, an Fc domain variant comprises a tryptophan (W) at amino acid position 373, a cysteine (C) at amino acid position 292, a cysteine (C) at amino acid position 302, an aspartic acid (D) at amino acid position 256, and a glutamine (Q) at amino acid position 307.

### Fc-Containing Binding Polypeptides

In one aspect, the current disclosure provides binding polypeptides (e.g., antibodies, antibody fragments, antibody variants, and fusion proteins) comprising an Fc domain (e.g., a variant Fc domain). In certain embodiments, the binding polypeptide is an antibody, or fragment or derivative thereof. Any antibody from any source or species can be employed in the binding polypeptides disclosed herein. Suitable antibodies include without limitation, human antibodies, humanized antibodies or chimeric antibodies.

Fc domains from any immunoglobulin class (e.g., IgM, IgG, IgD, IgA and IgE) and species can be used in the binding polypeptides disclosed herein. Chimeric Fc domains comprising portions of Fc domains from different species or Ig classes can also be employed. In certain embodiments, the Fc domain is a human IgG1 Fc domain.

In other embodiments, the current disclosure provides binding polypeptides (e.g., antibodies, antibody fragments, antibody variants, and fusion proteins) comprising at least one CH1 domain. CH1 domains from any immunoglobulin class (e.g., IgM, IgG, IgD, IgA and IgE) and species can be used in the binding polypeptides disclosed herein. Chimeric CH1 domains comprising portions of CH1 domains from different species or Ig classes can also be employed. In certain embodiments, the CH1 domain is a human IgG1 CH1 domain.

In one aspect, the present disclosure provides binding polypeptides comprising an isolated Fc domain variant comprising or complexed with (e.g., fused to) at least one binding domain (e.g., at least one binding polypeptide). In certain embodiments, the binding domain comprises one or more antigen binding domains. The antigen binding domains need not be derived from the same molecule as the parental Fc domain. In certain embodiments, the Fc domain variant is present in an antibody. In one embodiment, an Fc domain variant is present in an antibody or is complexed with an antibody. Any antibody from any source or species can be employed with an Fc domain variant disclosed herein. Suitable antibodies include without limitation, chimeric antibodies, humanized antibodies, or human antibodies. Suitable antibodies include without limitation, full-length antibodies, monoclonal antibodies, polyclonal antibodies, or immunoglobulin single variable domain antibodies, or VHHs.

The term “multispecific,” “multispecific antibody,” or “multispecific binding protein” can denote a binding protein that specifically binds two or more antigens. A multispecific binding protein that binds two antigens, and/or two different epitopes of different antigens, is also referred to herein as a “bispecific” binding protein. A multispecific binding protein that binds three antigens, and/or three different epitopes, is also referred to herein as a “trispecific” binding protein. Thus, the multispecific binding protein is able to bind two or more different targets simultaneously. Genetic engineering can be used to design, modify, and produce the multispecific binding protein, or a binding fragment or derivative thereof with a desired set of binding properties and effector functions.

In one aspect, a binding polypeptide composition described herein is an antibody. In some embodiments, the antibody is multispecific. In some embodiments, the multispecific antibodies are of a format selected from the group consisting of: a DVD-Ig, a CODV based format that is optionally a CODV-Ig, CrossMab, a CrossMab-Fab, and a Tandem Fabs. In some embodiments, the multispecific antibody is a T cell engager multispecific. In some embodiments, the multispecific antibody is a NK cell engager.

In certain embodiments, the binding polypeptide of the current disclosure may comprise an antigen binding fragment of an antibody. The term “antigen binding fragment” refers to a polypeptide fragment of an immunoglobulin or antibody which binds antigen or competes with intact antibody (i.e., with the intact antibody from which they were derived) for antigen binding (i.e., specific binding). Antigen binding fragments can be produced by recombinant or biochemical methods that are well known in the art.

Exemplary antigen binding fragments include a variable fragment (Fv), a Fab, a Fab′, a (Fab′)2, a minibody, a diabody, a triabody, a tetrabody, a tandem di-scFv, a tandem tri-scFv, an immunoglobulin single variable domain (ISV) such as, a VHH (including humanized VHH), a camelized VHH, a single domain antibody, a domain antibody, or a dAb.

In certain exemplary embodiments, a binding polypeptide of the current disclosure comprises at least one antigen binding fragment and an Fc domain variant. In certain exemplary embodiments, a binding polypeptide of the current disclosure comprises: (a) at least one antigen binding fragment selected from a group consisting of: a variable fragment (Fv), a Fab, a Fab′, a (Fab′)2, a minibody, a diabody, a triabody, a tetrabody, a tandem di-scFv, a tandem tri-scFv, an immunoglobulin single variable domain (ISV) such as, a VHH (including humanized VHH), a camelized VHH, a single domain antibody, a domain antibody, or a dAb; and (b) an Fc domain variant.

In exemplary embodiments, the binding polypeptide comprises a single chain variable region sequence (ScFv). Single chain variable region sequences comprise a single polypeptide having one or more antigen binding sites, e.g., a VL domain linked by a flexible linker to a VH domain. ScFv molecules can be constructed in a VH-linker-VL orientation or VL-linker-VH orientation. The flexible hinge that links the VL and VH domains that make up the antigen binding site includes from about 10 to about 50 amino acid residues. Connecting peptides are known in the art. Binding polypeptides may comprise at least one scFv and/or at least one constant region. In one embodiment, a binding polypeptide of the current disclosure may comprise at least one scFv linked or fused to an Fc domain variant.

In certain exemplary embodiments, a binding polypeptide of the current disclosure is a multivalent (e.g., tetravalent) antibody which is produced by fusing a DNA sequence encoding an antibody with a ScFv molecule (e.g., an altered ScFv molecule). For example, in one embodiment, these sequences are combined such that the ScFv molecule (e.g., an altered ScFv molecule) is linked at its N-terminus or C-terminus to an Fc domain variant via a flexible linker (e.g., a gly/ser linker). In another embodiment a tetravalent antibody of the current disclosure can be made by fusing an ScFv molecule to a connecting peptide, which is fused to an Fc domain variant to construct an ScFv-Fab tetravalent molecule.

In another embodiment, a binding polypeptide of the current disclosure is an altered minibody. An altered minibody of the current disclosure is a dimeric molecule made up of two polypeptide chains each comprising an ScFv molecule which is fused to an Fc domain variant via a connecting peptide. Minibodies can be made by constructing an ScFv component and connecting peptide components using methods described in the art (see, e.g., U.S. Pat. No. 5,837,821 or WO 94/09817AI). In another embodiment, a tetravalent minibody can be constructed. Tetravalent minibodies can be constructed in the same manner as minibodies, except that two ScFv molecules are linked using a flexible linker. The linked scFv-scFv construct is then joined to an Fc domain variant.

In another embodiment, a binding polypeptide of the current disclosure comprises a diabody. Diabodies are dimeric, tetravalent molecules each having a polypeptide similar to scFv molecules, but usually having a short (less than 10, e.g., about 1 to about 5) amino acid residue linker connecting both variable domains, such that the VL and VH domains on the same polypeptide chain cannot interact. Instead, the VL and VH domain of one polypeptide chain interact with the VH and VL domain (respectively) on a second polypeptide chain (see, for example, WO 02/02781). Diabodies of the current disclosure comprise an scFv-like molecule fused to an Fc domain variant.

In another embodiment, a binding polypeptide of the current disclosure comprises an immunoglobulin single variable domain (ISV), such as a domain antibody, a “dAb,” a VHH (including a humanized VHH), a camelized VHH, other single variable domains, or any suitable fragment of any one thereof, fused to an Fc domain variant.

The term “immunoglobulin single variable domain” (ISV or ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e., a total of 6 CDRs will be involved in antigen binding site formation. ISVs of the so-called “V_{H}3 class” (i.e., ISVs with a high degree of sequence homology to human germline sequences of the V_{H}3 class such as DP-47, DP-51 or DP-29) or ISVs belonging to the so-called “VH4 class” (i.e., ISVs with a high degree of sequence homology to human germline sequences of the V_{H}4 class such as DP-78), as for example described in WO 2007/118 670 A1, can be used herein.

The term “VHH” or “VHH antibody” is a type of single domain antibody that comprises variable heavy chain domains devoid of light chains. Similar to conventional VH domains, VHHs contain four FRs and three CDRs. VHHs have advantages over conventional antibodies. As they are about ten times smaller than lgG molecules, properly folded functional VHHs can be produced by in vitro expression while achieving high yield. Furthermore, VHHs are very stable, and resistant to the action of proteases. The properties and production of VHHs have been reviewed by Harmsen and De Haard H J (Appl. Microbiol. Biotechnol. 2007 November; 77 (1): 13-22).

In certain exemplary embodiments, a binding polypeptide of the disclosure comprises an Fc domain (e.g., an Fc domain variant) fused with one or more VHHs.

ISVs (in particular VHH sequences and partially humanized VHHs) can in particular be characterized by the presence of one or more “Hallmark residues” (as described herein in Table 1 and in subsequent paragraphs describing NANOBODY® immunoglobulin single variable domains) such that the ISV is a NANOBODY® ISV.

Thus, generally, a NANOBODY® ISV (in particular a VHH, including (partially or fully) humanized V_{HH} and camelized VH) can be defined as an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which one or more of the Hallmark residues are as further defined in Table 1. In particular, a NANOBODY® ISV (in particular a V_{HH}, including (partially) humanized VHH and camelized V_{H}) can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which the framework sequences are as further defined herein. More in particular, an ISV can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively.

The term “immunoglobulin single variable domain (ISV)” encompasses a NANOBODY® VHH as described in or WO 08/020079 or WO 09/138519, and thus in an aspect denotes a VHH, a humanized VHH or a camelized VH (such as a camelized human VH) or generally a sequence optimized VHH (such as e.g., optimized for chemical stability and/or solubility, maximum overlap with known human framework regions and maximum expression).

Generally, NANOBODY® immunoglobulin single variable domains (ISVs) (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISV can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in Nanobody ® ISVs.

| Position | Human V_{H}3 | Hallmark Residues |
|---|---|---|
| 11 | L, V; | L, S, V, M, W, F, T, Q, E, A, R, G, K, Y, N, P, I; |
| | predominantly L | preferably L |
| 37 | V, I, F; | F⁽¹⁾, Y, V, L, A, H, S, I, W, C, N, G, D, T, P, preferably |
| | usually V | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾;most preferably G⁽²⁾ or Q⁽³⁾. |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, |
| | | Q, N, D; preferably W⁽²⁾, L⁽¹⁾ or F⁽¹⁾ |
| 83 | R or K; | R, K⁽⁵⁾, T, E⁽⁵⁾, Q, N, S, I, V, G, M, L, A, D, Y, H; |
| | usually R | preferably K or R; most preferably K |
| 84 | A, T, D; | P⁽⁵⁾, S, H, L, A, V, I, T, F, D, R, Y, N, Q, G, E; preferably |
| | predominantly A | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g., as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

In other embodiments, the binding polypeptides comprise multispecific or multivalent antibodies comprising one or more variable domain in series on the same polypeptide chain, e.g., tandem variable domain (TVD) polypeptides. Exemplary TVD polypeptides include the “double head” or “Dual-Fv” configuration described in U.S. Pat. No. 5,989,830. In the Dual-Fv configuration, the variable domains of two different antibodies are expressed in a tandem orientation on two separate chains (one heavy chain and one light chain), wherein one polypeptide chain has two VH domains in series separated by a peptide linker (VH1-linker-VH2) and the other polypeptide chain consists of complementary VL domains connected in series by a peptide linker (VL1-linker-VL2). In the cross-over double head configuration, the variable domains of two different antibodies are expressed in a tandem orientation on two separate polypeptide chains (one heavy chain and one light chain), wherein one polypeptide chain has two VH domains in series separated by a peptide linker (VH1-linker-VH2) and the other polypeptide chain consists of complementary VL domains connected in series by a peptide linker in the opposite orientation (VL2-linker-VL1). Additional antibody variants based on the “Dual-Fv” format include the Dual-Variable-Domain IgG (DVD-IgG) bispecific antibody (see U.S. Pat. No. 7,612,181 and the TBTI format (see US 2010/0226923 A1). The addition of constant domains to respective chains of the Dual-Fv (CH1-Fc to the heavy chain and kappa or lambda constant domain to the light chain) leads to functional bispecific antibodies without any need for additional modifications (i.e., obvious addition of constant domains to enhance stability). In some embodiments, binding polypeptides comprise multi-specific or multivalent antibodies comprising one or more variable domain in series on the same polypeptide chain fused to an Fc domain variant.

In another exemplary embodiment, the binding polypeptide comprises a cross-over dual variable domain IgG (CODV-IgG) bispecific antibody based on a “double head” configuration (see US20120251541 A1, which is incorporated by reference herein in its entirety). CODV-IgG antibody variants have one polypeptide chain with VL domains connected in series to a CL domain (VL1-L1-VL2-L2-CL) and a second polypeptide chain with complementary VH domains connected in series in the opposite orientation to a CH1 domain (VH2-L3-VH1-L4-CH1), where the polypeptide chains form a cross-over light chain-heavy chain pair. In certain embodiment, the second polypeptide may be further connected to an Fc domain (VH2-L3-VH1-L4-CH1-Fc). In certain embodiments, linker L3 is at least twice the length of linker L1 and/or linker L4 is at least twice the length of linker L2. For example, L1 and L2 may be 1-3 amino acid residues in length, L3 may be 2 to 6 amino acid residues in length, and L4 may be 4 to 7 amino acid residues in length. Examples of suitable linkers include a single glycine (Gly) residue; a diglycine peptide (Gly-Gly); a tripeptide (Gly-Gly-Gly); a peptide with four glycine residues (Gly-Gly-Gly-Gly); a peptide with five glycine residues (Gly-Gly-Gly-Gly-Gly); a peptide with six glycine residues (Gly-Gly-Gly-Gly-Gly-Gly); a peptide with seven glycine residues (Gly-Gly-Gly-Gly-Gly-Gly-Gly); a peptide with eight glycine residues (Gly-Gly-Gly-Gly-Gly-Gly-Gly-Gly). Other combinations of amino acid residues may be used such as the peptide Gly-Gly-Gly-Gly-Ser and the peptide Gly-Gly-Gly-Gly-Ser-Gly-Gly-Gly-Gly-Ser.

In other embodiments, a binding polypeptide comprises a CrossMab or a CrossMab-Fab multispecific format. See WO2009080253 and Schaefer, et al., PNAS (2011), 108:11187-1191. Antibody variants based on the CrossMab format have a crossover of antibody domains within one arm of a bispecific IgG antibody enabling correct chain association. In some embodiments, a binding polypeptide comprises a Tandem Fab format. Tandem Fabs are a class of Fab-based bispecific antibody fragments. A tandem Fab comprises two Fabs targeting different epitopes.

In certain embodiments, the binding polypeptide comprises an immunoadhesin molecule comprising a non-antibody binding region (e.g., a receptor, ligand, or cell-adhesion molecule) fused to an antibody constant region (see e.g., Ashkenazi et al. (1995), Methods, vol. 8 (2), 104-115, which is incorporated by reference herein in its entirety).

In certain embodiments, the binding polypeptide comprises immunoglobulin-like domains. Suitable immunoglobulin-like domains include, without limitation, fibronectin domains (see, e.g., Koide A and Koide S (2007), Methods Mol. Biol. 352:95-109, which is incorporated by reference herein in its entirety), DARPin (see, e.g., Stumpp M T et al. (2008), Drug Discov. Today, vol. 13 (15-16): 695-701, which is incorporated by reference herein in its entirety), Z domains of protein A (see, e.g., Nygren P et al. (2008), FEBS J., vol. 275 (11): 2668-76, which is incorporated by reference herein in its entirety), Lipocalins (see, e.g., Skerra A (2008), FEBS J., vol. 275 (11): 2677-83, which is incorporated by reference herein in its entirety), Affilins (see, e.g., Ebersbach H et al. (2007), J. Mol. Biol., vol. 372 (1): 172-85, which is incorporated by reference herein in its entirety), Affitins (see, e.g., Krehenbrink M et al. (2008), J. Mol. Biol., vol. 383 (5): 1058-68, which is incorporated by reference herein in its entirety), Avimers (see, e.g., Silverman J et al. (2005), Nat. Biotechnol., vol. 23 (12): 1556-61, which is incorporated by reference herein in its entirety), Fynomers (see, e.g., Grabulovski D et al. (2007), J Biol Chem, vol. 282 (5): 3196-3204, which is incorporated by reference herein in its entirety), and Kunitz domain peptides (see, e.g., Nixon et al. (2006), Curr Opin Drug Discov Devel, vol. 9 (2): 261-8, which is incorporated by reference herein in its entirety).

In other embodiments, the binding polypeptide comprises a multispecific antibody in a T cell engager format. A “T cell engager” refers to binding proteins directed to a host's immune system, more specifically the T cells' cytotoxic activity as well as directed to a tumor target protein. In some embodiments, the isolated effector-competent polypeptide comprises a multispecific antibody in an NK cell engager format. An “NK cell engager” refers to binding proteins comprising monoclonal antibody fragments targeting activating NK cell receptors, antigen-specific targeting regions, and an Fc region (Gauthier L et al. (2019), Cell, vol. 177 (7): 1701-13).

A binding polypeptide of the present disclosure, comprising an Fc domain variant described herein, can include the CDR sequences or the variable domain sequences of a known “parent” antibody. In some embodiments, the parent antibody and the antibody of the disclosure can share similar or identical sequences except for modifications to the Fc domain as disclosed herein.

In another embodiment, the binding polypeptide comprises a therapeutic polypeptide. In some embodiments, the therapeutic polypeptide may be a receptor, a ligand, or an enzyme. In some embodiments, the therapeutic polypeptide may be a clotting factor. In some embodiments, the clotting factor is selected from the group consisting of FI, FII, FIII, FIV, FV, FVI, FVII, FVIII, FIX, FX, FXI, FXII, FXIII), VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In some embodiments, the therapeutic polypeptide may be a growth factor. The growth factor can be selected from any growth factor known in the art. In some embodiments, the growth factor is a hormone, in other embodiments, the growth factor is a cytokine. In some embodiments, the growth factor is a chemokine. In some embodiments, the binding polypeptide comprises a therapeutic molecule or therapeutic polypeptide linked to the N-terminus and/or the C-terminus of the Fc domain of the present invention. In some embodiments, the polypeptide is an Fc-fusion polypeptide.

In one embodiment, the binding polypeptides disclosed herein may be internalized by the cell. In another embodiment, the amount of the binding polypeptide internalized by the cell is greater than the amount of a reference binding polypeptide lacking a targeting moiety internalized by the cell.

In one embodiment, the targeting moiety binds to a receptor on the target cell. For example, the targeting moiety may comprise a mannose 6 phosphate moiety that binds to a mannose 6 phosphate receptor on the cell. In other exemplary embodiments, the targeting moiety binds to a Siglec on a target cell. Exemplary Siglecs include sialoadhesin (Siglec-1), CD22 (Siglec-2), CD33 (Siglec-3), MAG (Siglec-4), Siglec-5, Siglec-6, Siglec-7, Siglec-8, Siglec-9, Siglec-10, Siglec-11, Siglec-12, Siglec-14, or Siglec-15. In yet other embodiments, the targeting moiety comprises an α2,3-, α2,6-, or α2,8-linked sialic acid residue. In a further embodiment, the targeting moiety comprises an α2,3-siallylactose moiety or an α2,6-siallylactose moiety. Other exemplary receptors include lectin receptors, including but not limited to C-type lectin receptors, galectins, and L-type lectin receptors. Exemplary lectin receptors include: TDEC-205, macrophage mannose receptor (MMR), Dectin-1, Dectin-2, macrophage-inducible C-type lectin (Mincle), dendritic cell-specific ICAM3-grabbing nonintegrin (DC-SIGN, CD209), DC NK lectin group receptor-1 (DNGR-1), Langerin (CD207), CD 169, a lectican, an asialoglycoprotein receptor, DCIR, MGL, a DC receptor, a collectin, a selectin, an NK-cell receptor, a multi-CTLD endocytic receptor, a Reg group (type VII) lectin, chondrolectin, tetranectin, polycystin, attractin (ATRN), eosinophil major basic protein (EMBP), DGCR2, Thrombomodulin, Bimlec, SEEC, and CB CP/Frem 1/QBRICK.

In certain embodiments, the binding polypeptide of the disclosure comprises an engineered reactive amino acid residue that is conjugated to a LYTAC via a reactive moiety. In further embodiments, a linker conjugates the engineered reactive amino acid residue to the LYTAC.

In certain embodiments, the region of the LYTAC capable of binding a cell surface lysosome targeting receptor comprises mannose-6-phosphate (M6P) or derivatives thereof, GalNAc (e.g., trivalent GalNAc), and glycopeptides. In certain embodiments, the cell surface lysosome targeting receptor comprises an asialoglycoprotein receptor (ASGPR), a mannose-6-phosphate receptor (M6PR) (including, but not limited to, a cation-independent M6PR), and a sialic acid-binding immunoglobulin-type lectin (Siglec).

### Expression of Binding Polypeptides

In one aspect, polynucleotides encoding the Fc domain variants and/or the binding polypeptides disclosed herein are provided. Methods of making an Fc domain variant and/or a binding polypeptide comprising expressing these polynucleotides are also provided.

Polynucleotides encoding the Fc domain variants and/or the binding polypeptides disclosed herein are typically inserted in an expression vector for introduction into host cells that may be used to produce the desired quantity of the claimed antibodies, therapeutic polypeptides, and Fc-fusion proteins. Accordingly, in certain aspects, the invention provides expression vectors comprising polynucleotides disclosed herein and host cells comprising these vectors and polynucleotides.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes (such as human genes) synthesized as discussed above.

In other embodiments the binding polypeptides featured in the present disclosure may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is incorporated by reference herein. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for expression of an Fc domain variant and/or a binding polypeptide is of eukaryotic or prokaryotic origin. In one embodiment, the host cell line used for expression of an Fc domain variant and/or a binding polypeptide is of bacterial origin. In one embodiment, the host cell line used for expression of an Fc domain variant and/or a binding polypeptide is of mammalian origin. Those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT™ cells) (Biowa, Princeton, NJ)). In one embodiment NS0 cells may be used. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding binding polypeptides can also be expressed non-mammalian cells such as bacteria or yeast or plant cells. In this regard it will be appreciated that various unicellular non-mammalian microorganisms such as bacteria can be transformed and are capable of being grown in cultures or by fermentation. Bacteria, which are susceptible to transformation, include members of the Enterobacteriaceae, such as strains of Escherichia coli or Salmonella, and Bacillaceae, such as Bacillus subtilis, Pneumococcus, Streptococcus, and Haemophilus influenzae. It will further be appreciated that, when expressed in bacteria a binding polypeptide can become part of inclusion bodies. The binding polypeptides must be isolated, purified, and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, e.g., is commonly used (Stinchcomb D T et al. (1979), Nature, 282:39-43; Kingsman et al. (1979), Gene, vol. 7:141-52; and Tschemper et al. (1980), Gene, vol. 10:157-66). This plasmid already contains the TRP1 gene which provides a selection marker for a mutant strain of yeast lacking the ability to grow in tryptophan, for example, ATCC No. 44076 or PEP4-1 (Jones E W (1977), Genetics, vol. 85 (1): 23-33). The presence of the trpl lesion as a characteristic of the yeast host cell genome then provides an effective environment for detecting transformation by growth in the absence of tryptophan.

Methods of Treatment with Binding Polypeptides

In one aspect, the invention provides methods of treating a disease or disorder in a subject in need thereof comprising administering to the subject an effective amount of a binding polypeptide disclosed herein. In certain embodiments, the present disclosure provides kits and methods for the treatment of diseases and disorders, e.g., cancer in a mammalian subject in need of such treatment.

The binding polypeptides of the current disclosure are useful in a number of different applications. For example, in one embodiment, the subject binding polypeptides are useful for reducing or eliminating cells bearing an epitope recognized by the binding domain of the binding polypeptide. In another embodiment, the subject binding polypeptides are effective in reducing the concentration of or eliminating soluble antigen in the circulation. In one embodiment, the binding polypeptides may reduce tumor size, inhibit tumor growth and/or prolong the survival time of tumor-bearing animals. In another embodiment, the subject binding polypeptides are effective as T-cell engagers. Accordingly, this disclosure also relates to a method of treating tumors in a human or other animal by administering to such human or animal an effective, non-toxic amount of modified antibody.

In another embodiment, the subject binding polypeptides are useful for the treatment of other disorders or diseases, including, without limitation, infectious diseases, autoimmune disorders/diseases, inflammatory disorders/diseases, lung diseases, neuronal or neurodegenerative diseases, liver diseases, diseases of the spine, diseases of the uterus, depressive disorders and the like. Non-limiting examples of infectious diseases include those caused by RNA viruses, e.g., Orthomyxoviridae (e.g., influenza), Paramyxoviridae (e.g., respiratory syncytial virus, parainfluenza virus, metapneumovirus), Rhabdoviridae (e.g., rabies virus), Coronaviridae (e.g., SARS-COV), Togaviridae (e.g., chikungunya virus), Retroviridae (e.g., HIV) or DNA viruses. Examples of infectious diseases also include, without limitation, bacterial infectious diseases, caused by, e.g., Staphylococcus aureus, Staphylococcus epidermidis, Enterococcus, Streptococcus, Escherichia coli, and other infectious diseases caused by fungus (e.g., Candida albicans) or parasites (e.g., malaria). Other infectious diseases include, without limitation, SARS, yellow fever, Lyme borreliosis, leishmaniasis, anthrax and meningitis. Exemplary autoimmune disorders include, but are not limited to, psoriasis and lupus. Accordingly, this disclosure relates to a method of treating various conditions that would benefit from using a subject effector-competent polypeptide having, e.g., enhanced half-life.

One skilled in the art would be able, by routine experimentation, to determine what an effective, non-toxic amount of binding polypeptide would be for the purpose of treating malignancies. For example, a therapeutically active amount of a binding polypeptide of the present disclosure may vary according to factors such as the disease stage (e.g., stage I versus stage IV), age, sex, medical complications (e.g., immunosuppressed conditions or diseases) and weight of the subject, and the ability of the modified antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In general, the compositions provided in the current disclosure may be used to prophylactically or therapeutically treat any neoplasm comprising an antigenic marker that allows for the targeting of the cancerous cells by the modified antibody.

### Pharmaceutical Compositions and Administration Thereof

Methods of preparing and administering binding polypeptides of the current disclosure to a subject can be readily determined by those skilled in the art. The route of administration of the binding polypeptides of the current disclosure may be oral, parenteral, by inhalation, topical, or any other suitable method. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in U.S. Pat. Publ. US 2002-0102208 and U.S. Pat. No. 6,994,840, each of which is incorporated herein by reference. Such articles of manufacture will typically have labels or package inserts indicating that the associated compositions are useful for treating a subject suffering from or predisposed to autoimmune or neoplastic disorders.

Effective doses of the compositions of the present disclosure, for the treatment of the conditions described above vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

Binding polypeptides of the current disclosure can be administered on multiple occasions. Intervals between single dosages can be weekly, monthly or yearly. Intervals can also be irregular as indicated by measuring blood levels of Fc domain variant or antigen in the patient. In some methods, dosage is adjusted to achieve a plasma modified binding polypeptide concentration of about 1-1000 μg/ml and in some methods about 25-300 μg/ml. Alternatively, binding polypeptides can be administered as a sustained release formulation, in which case less frequent administration is required. For antibodies, dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present polypeptides or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.1 to about 25 mg per dose, especially about 0.5 to about 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from about 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

Binding polypeptides of the current disclosure can optionally be administered in combination with other agents that are effective in treating the disorder or condition in need of treatment (e.g., prophylactic or therapeutic). Effective single treatment dosages (i.e., therapeutically effective amounts) of 90Y-labeled modified antibodies of the current disclosure range from between about 5 and about 75 mCi, such as between about 10 and about 40 mCi. Effective single treatment non-marrow ablative dosages of 1311-modified antibodies range from between about 5 and about 70 mCi, or between about 5 and about 40 mCi. Effective single treatment ablative dosages (i.e., may require autologous bone marrow transplantation) of 1311-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment non-marrow ablative dosages of iodine-131 labeled chimeric antibodies range from between about 5 and about 40 mCi, such as less than about 30 mCi. Imaging criteria for, e.g., the 111 In label, are typically less than about 5 mCi.

While the binding polypeptides may be administered as described immediately above, it must be emphasized that in other embodiments the polypeptide may be administered to otherwise healthy patients as a first line therapy. In such embodiments, the binding polypeptides may be administered to patients having normal or average red marrow reserves and/or to patients that have not, and are not, undergoing treatment. As used herein, the administration of the polypeptides in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment.

As previously discussed, the binding polypeptides of the present disclosure, antibodies, therapeutic polypeptides, or Fc domain variant-fusion polypeptides thereof, may be administered in a pharmaceutically effective amount for the in vivo treatment of mammalian disorders. In this regard, it will be appreciated that the disclosed Fc domain variants will be formulated to facilitate administration and promote stability of the active agent.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of the binding polypeptides, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the polypeptide can interact with selected antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the binding polypeptides of the disclosure may be administered to a human or other animal in accordance with the aforementioned methods of treatment in an amount sufficient to produce a therapeutic or prophylactic effect. The binding polypeptides of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The present invention is further illustrated by the following examples which should not be construed as further limiting. The contents of the Sequence Listing, figures and all references, patents, and published patent applications cited throughout this application are expressly incorporated herein by reference.

### Example 1. The Impact of Glycosylation on Effector Function of a Monoclonal Antibody with Fc-Mutation

### Introduction

Many efforts have been made to enhance ADCC for increasing the efficacy in the treatment against diseases. Protein engineering of the Fc-CH2 domain using site-directed mutagenesis can significantly increase FcγRIIIa binding and ADCC activity (Lazar G A et al. (2006), Proc. Natl. Acad. USA, vol. 103 (11): 4005-10 and Liu Z et al. (2014), J Biol Chem, vol. 289 (6): 3571-90.) Similarly, the Fc-glycoengineering, especially through core fucose removal, also led to significant ADCC enhancement (Shields R L et al. (2002), J Biol Chem, vol. 277 (30): 26733-40 and Shinkawa T et al. (2003), J Biol Chem., vol. 278 (5): 3466-73.) Both Fc engineering strategies resulted in 10 to 100-fold enhanced potency (Masuda K et al. (2007), Mol Immunol, vol. 44 (12): 3122-31.) In this study, whether a double mutation, S239D/I332E (DE mutation), combined with different Fc-glycosylation, including afucosylation, mannosylation, and hypergalactosylation, would have any further impact on both FcγRIIIa binding and ADCC was studied. The results presented here demonstrate synergies in effector function of a recombinant human monoclonal IgG1 antibody, mAb A with combined Fc-mutation and glycan modification.

### Materials and Methods

Cell culturing, antibody expression/modification, and SDS-PAGE: The human monoclonal antibodies (IgG1) were produced from Chinese hamster ovary (CHO) cells with wild-type (WT) Fc sequence and DE mutation (S239D/I332E) (Lazar G A et al. (2006), Proc. Natl. Acad. USA, vol. 103 (11): 4005-10.) The CHO cells expressing antibody were cultured in suspension in CD-CHO containing 4 mM glutamine (ThermoFisher Scientific®). The afucosylated WT and DE antibodies were generated by transfecting the antibody expressing CHO cells with the genes coding for bacterial oxidoreductase GDP-6-deoxy-D-lyxo-4-hexulose reductase (RMD) and green fluorescent protein (GFP). The transfection was performed using Gene Pulser Xcell electroporation system (Bio-Rad®) before the transfectants were selected in G418. The RMD expressing cells were then sorted through the GFP fluorescence by using flow cytometry (von Horsten H H, et al. Production of non-fucosylated antibodies by co-expression of heterologous GDP-6-deoxy-D-lyxo-4-hexulose reductase Glycobiology 2010; 20 (12): 1607-18.) The afucosylated antibody was also generated by treating the antibody-expressing cells with 1,3,4-Tri-O-acetyl-2-deoxy-2-fluoro-L-fucose, peracetylated 2-fluoro 2-deoxy-L-fucose (2FF, 0 to 200 UM) (Okeley N M et al. (2013), Proc. Natl. Acad. USA, vol. 110 (14): 5404-9.) The antibody containing oligomannose was generated by treating cells with kifunensine, a potent α-mannosidase I inhibitor (Zhou Q et al. (2008), Biotechnol Bioeng, vol. 99 (3): 652-65.) Kifunensine (2 μg/ml) was added into the antibody expressing cell cultures at day 0, and the cells grew for 11 days before they were harvested for antibody purification. β-galactosyltransferase was used to prepare hypergalactosylated antibody in vitro (Houde D et al. (2010), Mol Cell Proteomics, vol. 9 (8): 1716-28.)

Antibody IgG1 was purified by using Protein A chromatography. Briefly, Protein A affinity media, MAbSelect (GE Healthcare®) was equilibrated with PBS (pH 7.2). The column was loaded with samples and washed with 10 column volumes of equilibration buffer, then eluted with 12.5 mM citric acid, and pH was adjusted immediately to ˜7.0 with 0.5 M HEPES buffer (pH 7.2). The antibodies were buffer exchanged for five times into PBS (pH 7.2). They were analyzed using SDS-PAGE under reducing and non-reducing condition with 4-12% NuPAGE (ThermoFisher Scientific®.)

N-linked glycan analysis: N-linked glycans were released from antibodies with PNGase F and purified using solid-phase extraction. Aliquots of samples were mixed with 2,5-dihydroxybenzoic acid matrix and were applied to a target. MALDI-TOF mass spectra were acquired using a Brukar Autoflex™ Speed MALDI-TOF (Bruker Daltonics, Billerica, MA) in the positive-ion reflectron mode. Some of the released N-linked glycans were also labeled with 2-aminobenzamide (2-AB) and analyzed using hydrophilic interaction liquid chromatography-ultra high-performance liquid chromatography (HILIC-UPLC) (Reusch D et al. (2015), MAbs, vol. 7 (1): 167-79.) The glycans were separated using a glycan BEH amide with glycan BEH amide column (2.1×150 mm) on an Acquity UPLC® H-class Bio System (Waters®). The column was equilibrated in 25% solvent A (50 mM ammonium formate, pH 4.4) and 75% solvent B (100% acetonitrile). The 2-AB labeled glycans were eluted at 60° C. using a linear gradient of 75-0% solvent B over 36.5 min at a flow rate of 0.4 to 0.2 mL/minute.

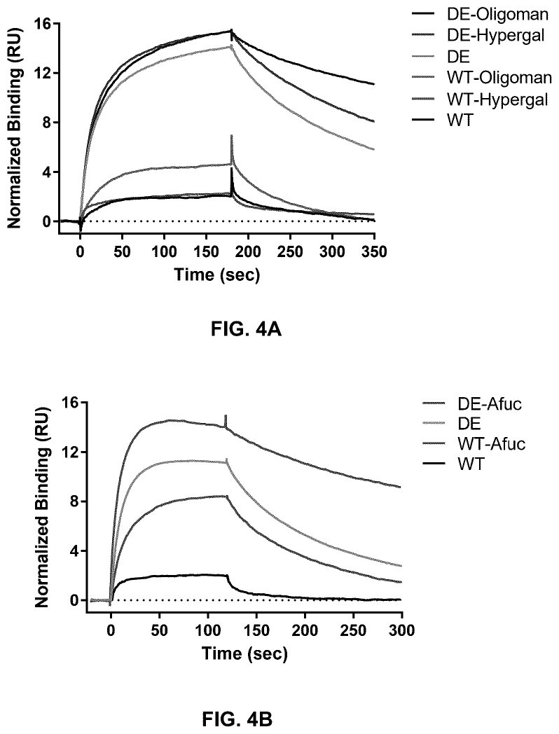

FcγRilla Binding Analysis: Analysis of antibody binding to recombinant human FcγRIIIa-V158 was performed using surface plasmon resonance (SPR) on a Biacore™ T200 instrument. Antibodies were diluted to 5 μg/mL in HBS-EP+ (10 mM HEPES pH 7.4, 150 mM NaCl, 3 mM EDTA, 0.05% surfactant P20) and injected over a sensor chip immobilized with Protein A (Cytiva) for 30 sec at 10 μL/min flowrate at 25° C. to obtain capture levels between. Recombinant human FcγRIIIa-V158 was serially diluted 3-fold from 900 to 3.7 nM (100 nM shown in FIG. 4A-B) in running buffer and injected over the captured antibodies for 2 min in duplicate followed by 3 min dissociation in buffer at 30 μL/min flowrate. The surface was regenerated with 10 mM glycine pH 1.5 for 30 sec. Sensorgrams were processed using the BiaEvaluation software (GE Healthcare®) and fit to a 1:1 binding model to calculate binding affinity (KD).

ADCC activity assay: The ADCC potency was measured using a bioluminescent reporter bioassay (Promega™). This method utilizes target cells which express the cell surface target antigen and effector cells, Jurkat cells, engineered to express the FcγRIIIa (V158) and a luciferase reporter. In the presence of target cells, antibody and the engineered effector cells, pathway activation leads to induction of a luciferase reporter in the effector cells. Luciferase production is proportional to the level of ADCC activity present. A similar assay has been reported as an alternative method to measure ADCC without the isolation of peripheral blood mononuclear cells (PBMCs) from fresh blood (Parekh B S et al. (2012), MAbs, vol. 4 (3): 310-8 and Kurogochi M et al. (2015), PloS One, vol. 10 (7): e0132848.) It has been shown to correlated well with standard approach using PBMC with good accuracy, precision and robustness (Parekh B S et al. (2012), MAbs, vol. 4 (3): 310-8.)

Multiple doses of the antibody are added to the target cells in duplicate. Effector cells were then added to the assay plate containing target cells and antibody. After a set incubation, luciferase substrate was added and the reaction is measured for luciferase production. The luciferase responses for each dose of the sample and reference material are fit to a four parameter model. Dose response curves for the sample are compared to those of the reference material. Due to the fact that various molecules were compared for these studies, not all curves were parallel to the reference material, or to each other. Therefore, an estimated fold increase in ADCC activity was determined for molecule comparison.

### Results

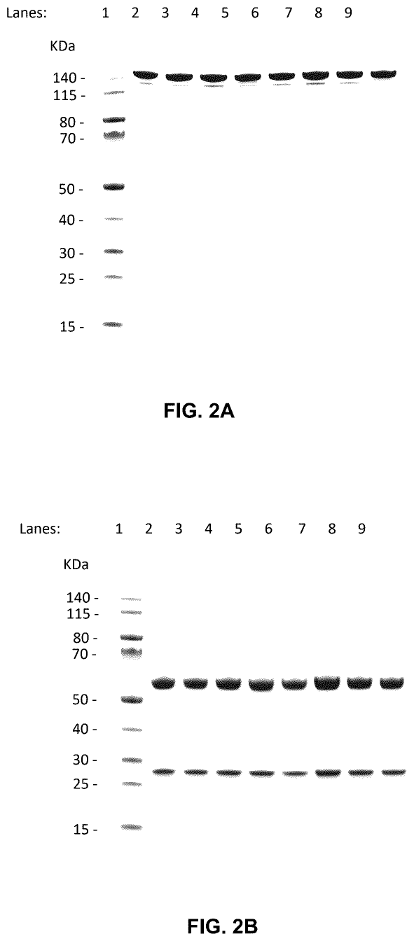

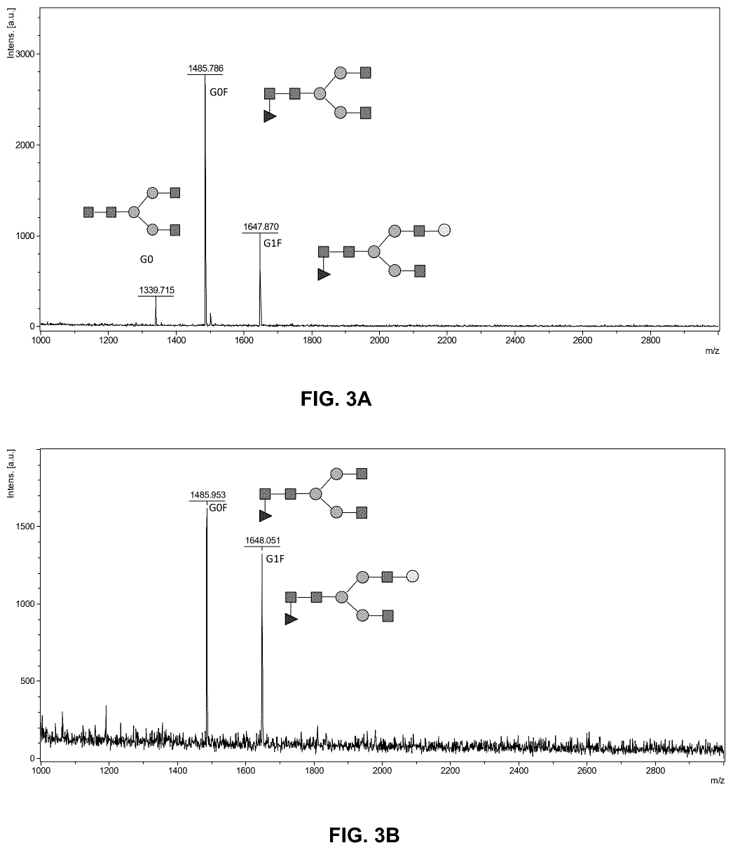

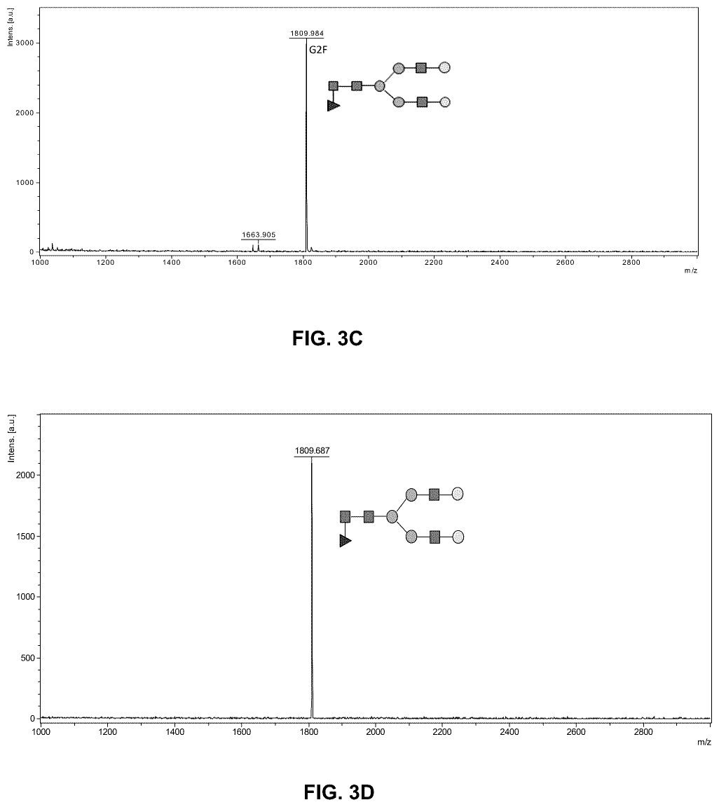

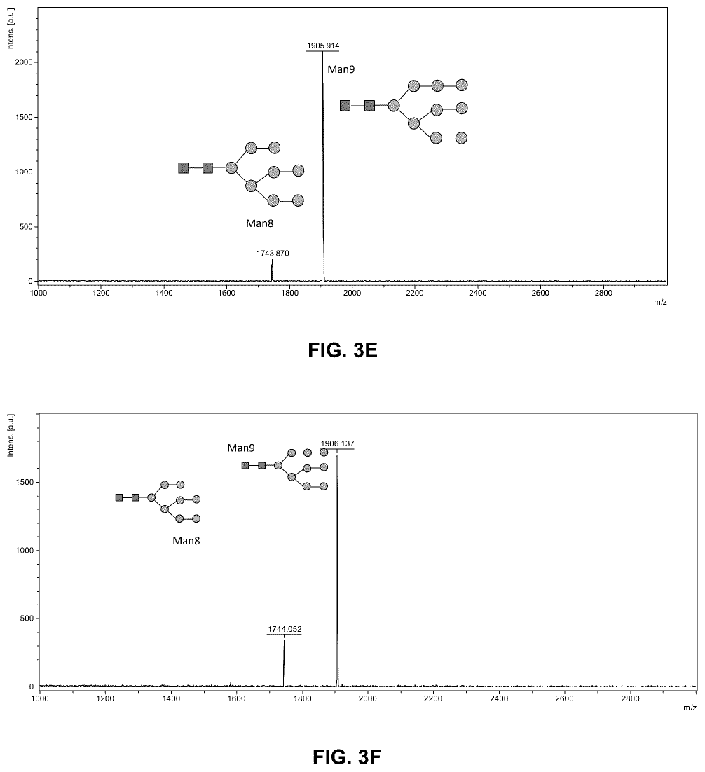

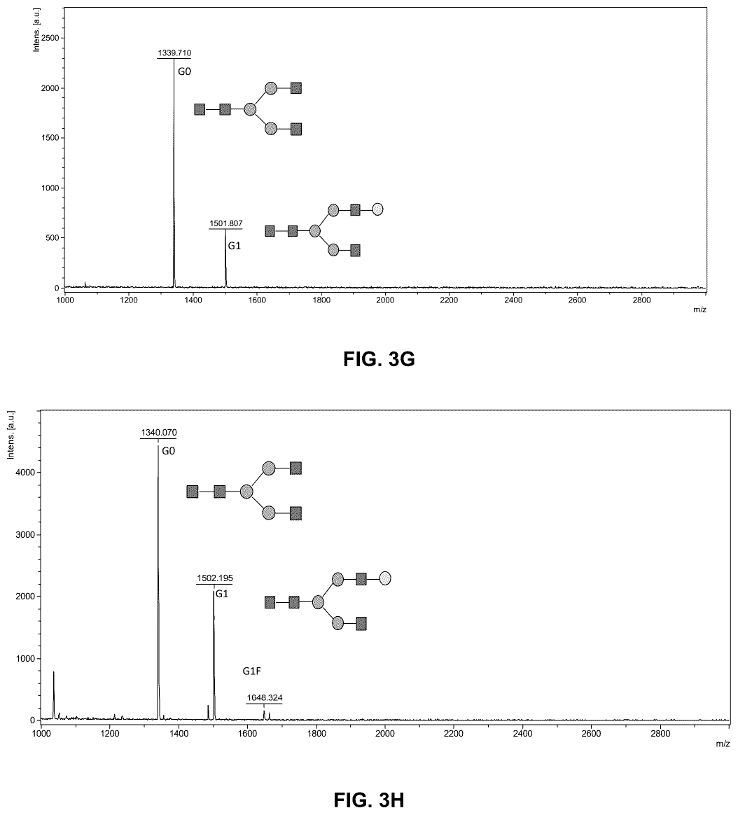

Antibody Fc engineering for enhanced effector functions: The N-linked glycans in wild-type (WT) and DE mutant antibodies specific for protein 1 were modified enzymatically, metabolically, or recombinantly to achieve hypergalactosylation, mannosylation, or afucosylation. As shown in FIG. 2A-B, the analysis of these antibodies using SDS-PAGE displayed expected antibody size and profiles with a minimal impurity or aggregate. Their N-linked glycans were released with PNGase F and analyzed using MALDI-TOF MS. As shown in FIG. 3A and FIG. 3B, the results demonstrate that both the WT and DE antibodies contain mainly G0F (GlcNAC₂Man₃GlcNAC₂Fuc₁) and G1F (Gal₁GlcNAC₂Man₃GlcNAc₂Fuc₁) glycans which are the major species found in most of recombinant human antibodies. As shown in FIG. 3C and FIG. 3D, the N-linked glycans on WT and DE mutant became hypergalactosylated species, G2F (Gal₂GlcNAC₂Man₃GlcNAC₂Fuc₁), when the antibody was modified in vitro with galactosyltransferase. As shown in FIG. 3E and FIG. 3F, the WT and DE antibodies had predominately oligomannose-type glycans, Man₉ (Man₉GlcNAC₂) and Man₈ (Man₈GlcNAC₂), when they were purified from kifunensine treated cell cultures. There were mainly afucosylated glycans, G0 (GlcNAC₂Man₃GlcNAc₂) and G1 (Gal₁GlcNAc₂Man₃GlcNAC₂), in the WT and DE antibodies purified from cells transfected with the gene coding for bacterial oxidoreductase GDP-6-deoxy-D-lyxo-4-hexulose reductase (RMD) (FIG. 3G and FIG. 3H.)

FcγRIIIa binding of Fc-engineered antibodies: The FcγRIIIa binding of WT and DE antibodies specific for protein 1 with different N-linked glycans were investigated using surface plasmon resonance (SPR). DE mutation resulted in the strongest interaction of the antibody with FcγRIIIa, when compared with the glycan-modified WT antibodies (Tables 1 and 2, shown below). The order of binding can be ranked as follows: DE>afucosylated WT>mannosylated WT>hypergalactosylated WT>WT. When the DE mutation was combined with glycan modification for hypergalactosylation or mannosylation, there was a synergistic effect on increasing FcγRIIIa binding (FIG. 4A). The antibodies with hypergalactose and oligomannose displayed 1.8 and 2.3-fold higher affinity than the unmodified DE antibody, respectively, mainly due to reduced dissociation rate constants (k_{d}) (Table 1). When the DE mutation was combined with afucosylation, there was a 3.5-fold increase in the receptor binding of afucosylated DE compared to DE antibody (FIG. 4B). The enhanced interaction also mainly results from reduced dissociation rate (Table 2).

TABLE 1
Affinity and kinetic analysis of FcγRIIIa* binding
to WT and DE antibodies specific for protein 1
containing hypergalactose and oligomannose.

| | kₐ | k_{d} | K_{D} |
|---|---|---|---|
| Antibody | (×10⁵ M⁻¹s⁻¹) | (×10⁻²s⁻¹) | (nM) |
| WT | 0.6 | 3.1 | 540 |
| WT hypergalactosylated | 0.6 | 2.5 | 409 |
| WT oligomannose | 0.7 | 1.7 | 256 |
| DE | 4 | 0.6 | 14 |
| DE hypergalactosylated | 4.8 | 0.4 | 8 |
| DE oligmannose | 4 | 0.2 | 6 |

*FcγRIIIa (V158) was used in SPR analysis.

TABLE 2
Affinity and kinetic analysis of FcγRIIIa* binding to
WT and DE antibodies specific for protein 1 without fucose.

| | kₐ | k_{d} | K_{D} |
|---|---|---|---|
| Antibody | (×10⁵ M⁻¹s⁻¹) | (×10⁻²s⁻¹) | (nM) |
| WT | 1.9 | 10.7 | 557 |
| WT afucosylated | 3.7 | 1.1 | 30 |
| DE | 7.3 | 1 | 14 |
| DE afucosylated | 7.5 | 0.3 | 4 |

*FcγRIIIa (V158) was used in SPR analysis.

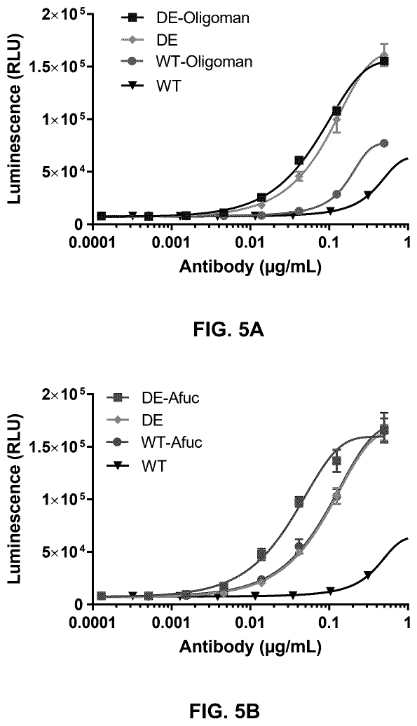

Synergistic effects of engineered antibodies on ADCC: The ADCC activity of the DE antibodies specific for protein 1 with glycan modifications was evaluated using an ADCC reporter gene assay. The DE mutation combined with mannosylation exhibits potential synergistic enhancement (FIG. 5A). The engineered DE antibody containing oligomannose showed slightly high ADCC activity (1.4-fold) compared to the DE antibody without modification. The ADCC activity for the DE antibody with or without hypergalactosylation was found to be similar.

Interestingly, although there is a higher FcγRIIIa affinity of DE mutant than afucosylated WT antibody (Table 2), their ADCC activities were found to be similar (FIG. 5B). Further, the DE mutation plus afucosylation resulted in synergistic enhancement in ADCC (FIG. 5B). There is approximately a 3.2-fold increase in ADCC activity as compared to the DE antibody without modification.

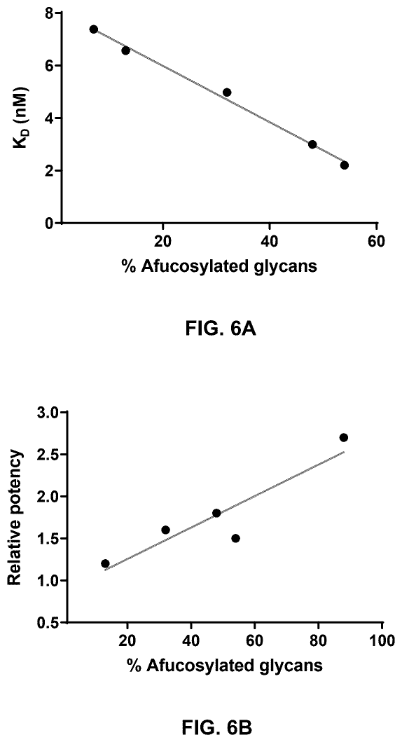

Correlation of effector function with afucosylation level in antibody with DE mutation: To better understand the impact of afucosylation with Fc-mutation on effector function, different amounts of afucosylation were generated by treating the DE antibody-expressing cells with a fucose analog, peracetylated 2-fluoro-fucose, for fucosyltransferase inhibition. For more accurate quantitation, N-linked glycans released from the antibodies were labeled with 2-AB, and analyzed using UPLC. As shown in Table 3 below, there was a reduction in α1,6-linked fucose in the glycans after the antibody purified from the cells treated with increasing amounts of the fucose analog. The effector functions of the antibodies were determined using FcγRIIIa binding and ADCC reporter gene assay. As shown in FIG. 6A, there was a linear correlation between the amounts of afucosylation and the receptor binding (r² =0.99). Furthermore, there was also a positive correlation of enhanced ADCC activity with increased amounts of afucosylation in the same antibody (r²=0.84) (FIG. 6B). The results suggest that lower fucose content leads to higher FcγRIIIa affinity and ADCC for the antibody in the context of DE mutation.

TABLE 3
UPLC analysis of N-linked glycans from antibodies generated from the
cells treated with peracetylated 2-fluoro 2-deoxy-L-fucose (2FF)

| μM | % | % | % | % | % | % | % |
|---|---|---|---|---|---|---|---|
| 2FF | G0 | G0F | G1 | G1F | G2 | G2F | Afucose |
| 0 | 3.8 | 41.2 | 1.8 | 43.1 | 1.3 | 8.8 | 7.0 |
| 12.5 | 7.3 | 38.9 | 4.6 | 40.1 | 1.4 | 7.7 | 13.3 |
| 25 | 18.0 | 29.6 | 11.8 | 32.1 | 1.7 | 6.8 | 31.5 |
| 50 | 28.0 | 22.2 | 17.6 | 24.6 | 2.4 | 5.2 | 48.0 |
| 100 | 30.9 | 19.2 | 20.0 | 22.2 | 2.8 | 4.9 | 53.7 |

### Conclusions

To better understand the impact of glycosylation on Fc-mutations, the effects of combined protein mutagenesis with glycan modifications were investigated. A glycan modification of a double mutation, DE mutation, was used. A human recombinant monoclonal antibody IgG1 with a double mutation (DE) was modified for afucosylation, mannosylation, and hypergalactosylation. Synergistic effects in FcγRIIIa binding and ADCC activity are demonstrated with the antibody containing both DE mutation and afucosylation. There are also synergies in FcγRIIIa binding of mannosylation and hypergalactosylation combined with the DE mutation. The increased interactions of FcγRIIIa with DE antibodies containing three different kinds of glycans are mainly due to the reduced dissociation rate. Different mechanisms likely contribute to the synergistic enhanced interaction of FcγRIIIa. Moreover, the enhanced effector function is correlated with the increased levels of afucosylation in the antibody. These results suggest that different glycosylation in antibody with Fc-mutation may have variable impact on receptor binding and ADCC activity.

Although there was minor effect on FcγRIIIa binding and no further enhanced ADCC with DE plus hypergalactosylation, the DE antibody containing oligomannose displayed greater receptor binding and slightly higher ADCC activity than the DE without modification.

In conclusion, these results show synergistic effects of glycosylation, including afucosylation, mannosylation and hypergalactosylation, on an antibody with effector function-enhancing Fc-mutations. Thus, these results provide the first evidence of a synergistic effect of n effector function-enhancing mutations in Fc domain plus Fc-glycoengineering.

### Example 2. Human FcγRIIIa Affinity of Variants of Antibodies Specific for Protein 2

### Introduction

In order to test for the synergistic effect on the enhanced effector function, Fc mutants of antibodies specific for protein 2, were produced with or without kifunensine treatment. Human FcγRIIIa binding affinity was measured after purification.

### Materials and Methods

FcγRilla Binding Analysis: FcγRIIIa binding analysis was performed using surface plasmon resonance (SPR) on a Biacore™ T200 instrument as described above in Example 1 with the following modifications. Antibodies were diluted to 0.5 μg/mL and recombinant human FcγRIIIa-V158 was serially diluted 3-fold from 3000 nM to 0.457 nM in HBS-EP+pH 7.4 running buffer and injected for 2 min at 50 μL/min. Sensorgrams were processed and fit using a 1:1 kinetic binding model.

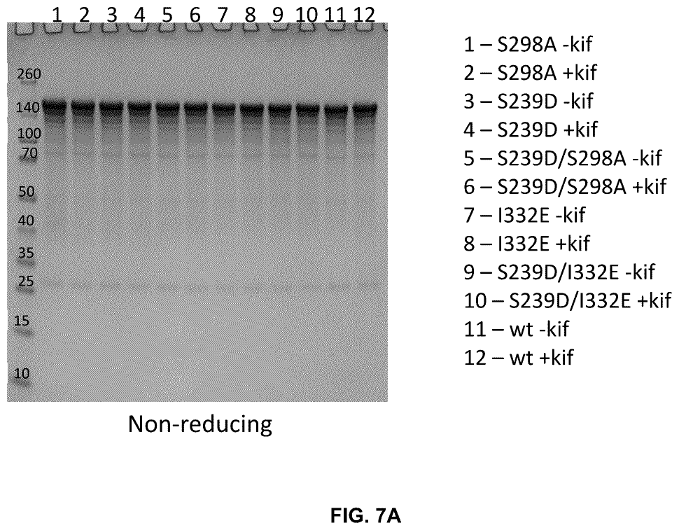

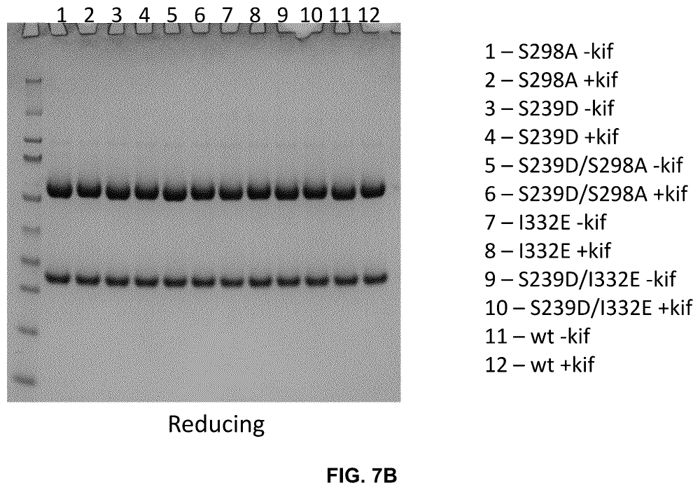

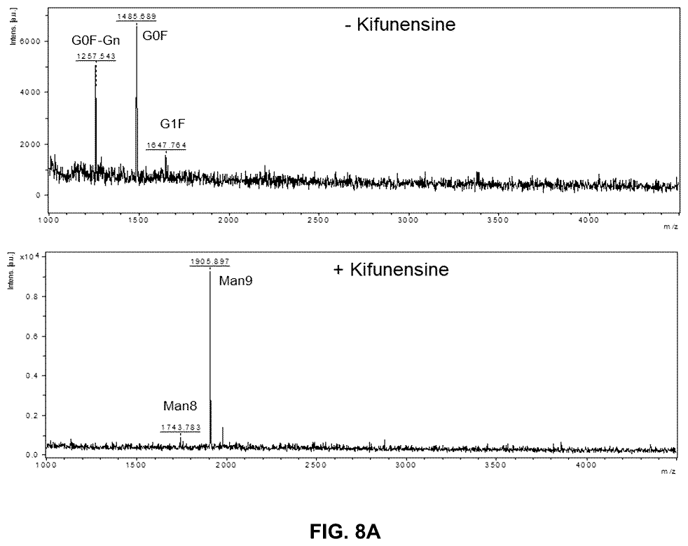

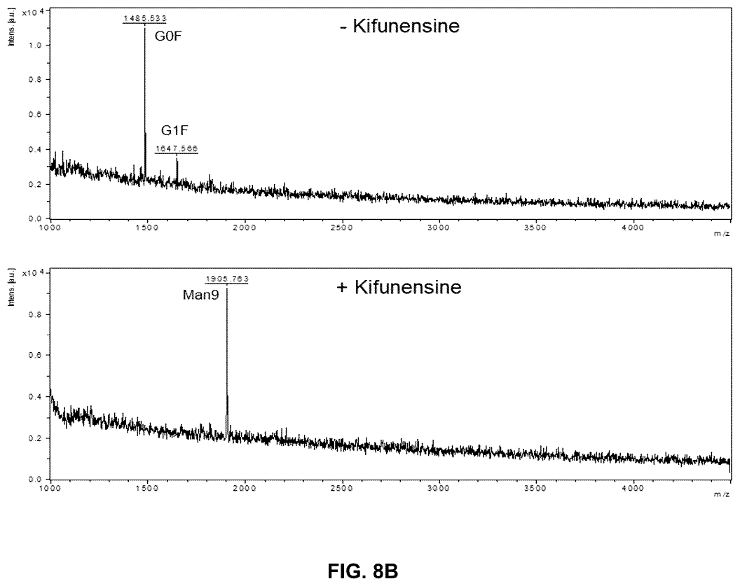

The expression and purification of variants of antibodies specific for protein 2 with or without kifunensine is shown in Table 4, below. The expression was done using Expi293™ expression medium (20 mL scale) with 2 μg/mL kifunensine. The purification was done using MabSelect Sure™ 1 mL columns with 5 column volumes (CV) single step elution and buffer-exchange was done on Amicon® units. FIG. 7A-B depicts SDS-PAGE of the Fc variants of antibodies specific for protein 2 with and without kifunensine treatment. FIG. 7A depicts non-reducing conditions. FIG. 7B depicts reducing conditions. 4-12% BT SDS-PAGE with MES buffer, 4 μg protein/lane was used.

TABLE 4
Expression and Purification of Variants of Antibodies
Specific for Protein 2 +/− Kifunensine

| | | Conc., | Volume, | Amount, | Titer, |
|---|---|---|---|---|---|
| | Variant | mg/mL | mL | mg | μg/mL |
| 1 | S298A (−kif) | 4.24 | 2 | 8.5 | 424 |
| 2 | S298A (+kif) | 3.73 | 2 | 7.5 | 373 |
| 3 | S239D (−kif) | 3.15 | 2.2 | 6.9 | 347 |
| 4 | S239D (+kif) | 3.13 | 2.3 | 7.2 | 360 |
| 5 | S239D/S298A (−kif) | 3.29 | 2 | 6.6 | 329 |
| 6 | S239D/S298A (+kif) | 3.24 | 2.4 | 7.8 | 389 |
| 7 | I332E (−kif) | 4.35 | 2 | 8.7 | 435 |
| 8 | I332E (+kif) | 3.62 | 2.3 | 8.3 | 416 |
| 9 | S239D/I332E (−kif) | 2.87 | 2.2 | 6.3 | 316 |
| 10 | S239D/I332E (+kif) | 2.96 | 2.3 | 6.8 | 340 |
| 11 | wt (+kif) | 2.63 | 2 | 5.3 | 263 |
| 12 | wt (−kif) | 1.11 | N/A | N/A | |

### Results

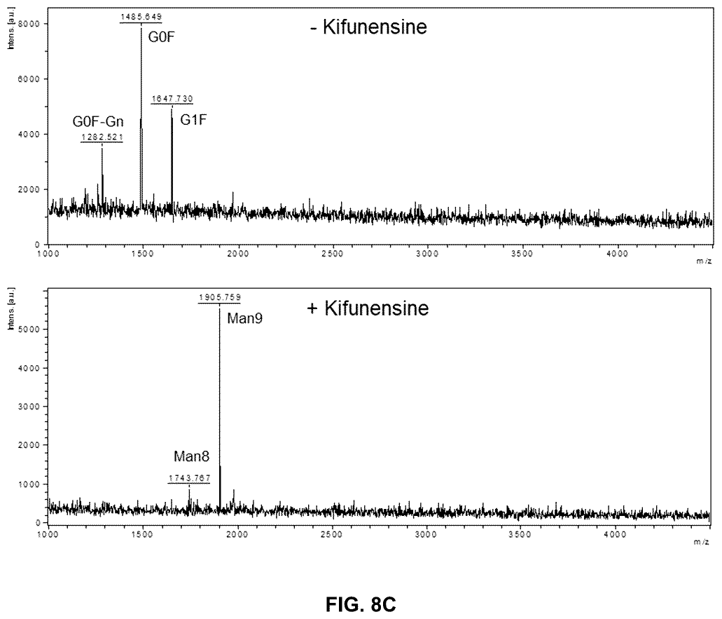

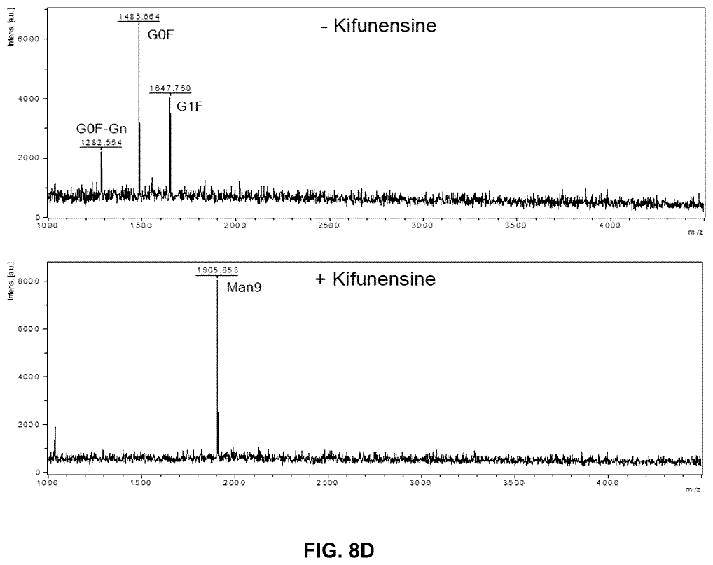

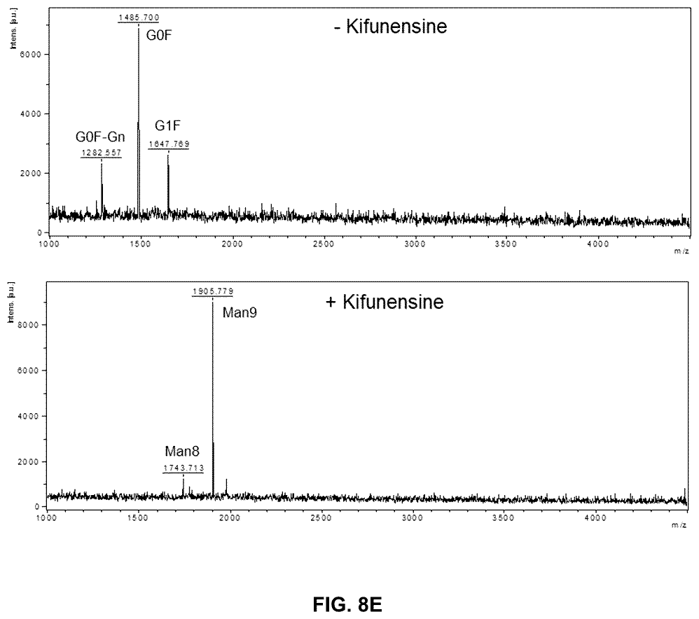

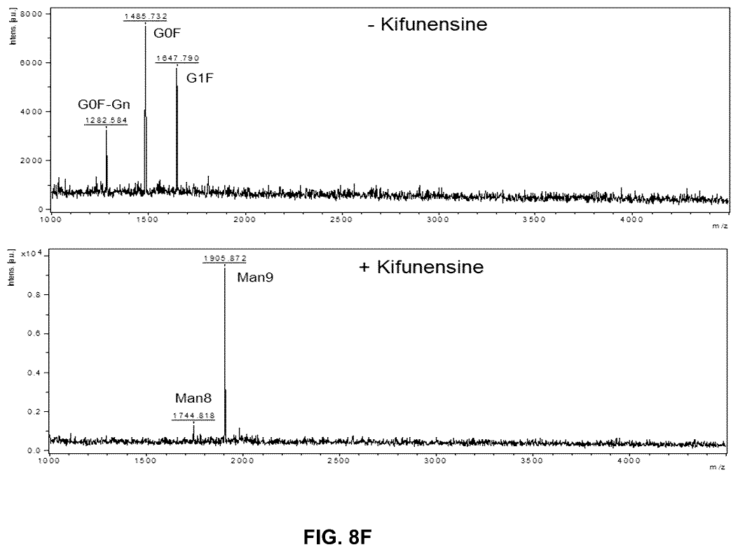

Glycan Analysis: An analysis of the glycan structures of the various Fc variant antibodies with and without kifunensine was done using MALDI-TOF and is presented in FIG. 8A-F. As shown in FIG. 8A, the wildtype antibody with predominately G0F-Gn, G0F, and G1F glycans without kifunensine treatment and Man8 and Man9 glycans with kifunensine treatment. As shown in FIG. 8B, the S298A antibody with predominately G0F and G1F glycans without kifunensine treatment and Man9 glycans with kifunensine treatment. As shown in FIG. 8C, the S239D antibody with predominately G0F-Gn, G0F, and G1F glycans without kifunensine treatment and Man8 and Man9 glycans with kifunensine treatment. As shown in FIG. 8D, the S239D/S298A antibody with predominately G0F-Gn, G0F, and G1F glycans without kifunensine treatment and Man9 glycans with kifunensine treatment. As shown in FIG. 8E, the I332E antibody with predominately G0F-Gn, G0F, and G1F glycans without kifunensine treatment and Man8 and Man9 glycans with kifunensine treatment. As shown in FIG. 8F, the S239D/I332E antibody with predominately G0F-Gn, G0F, and G1F glycans without kifunensine treatment and Man8 and Man9 glycans with kifunensine treatment.

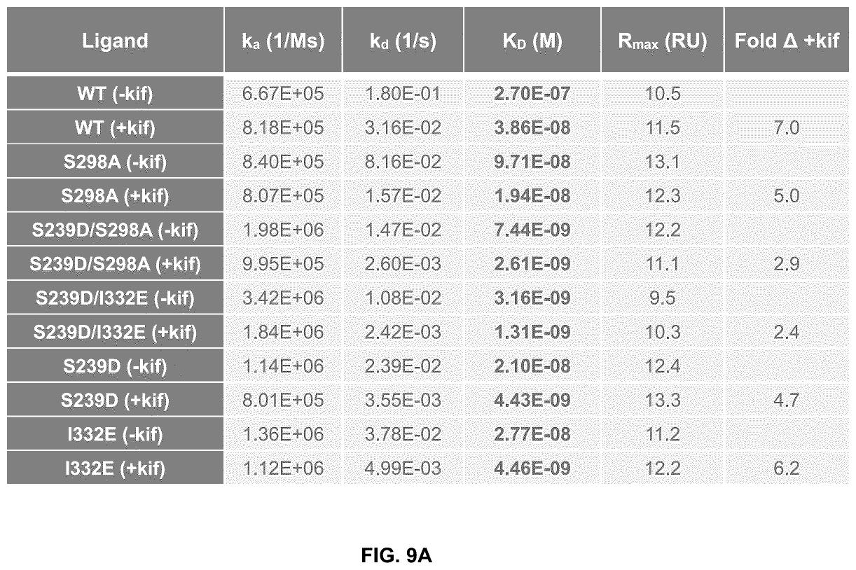

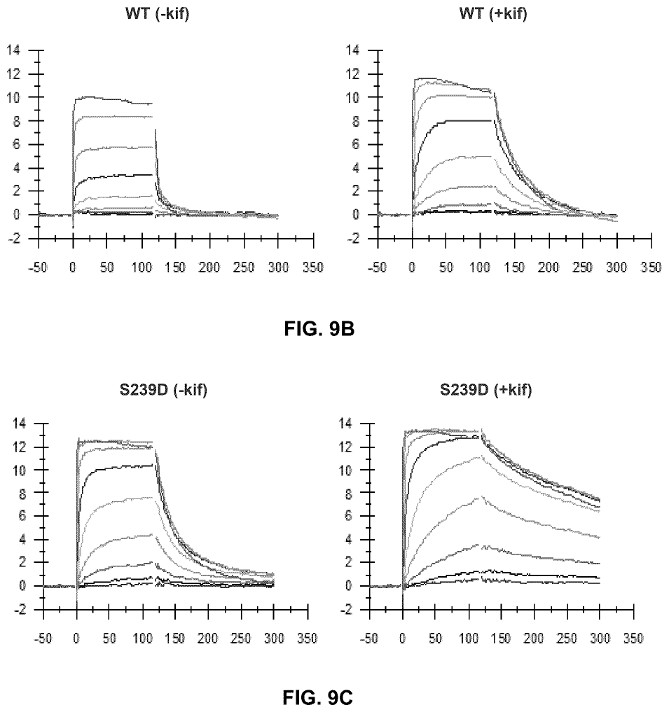

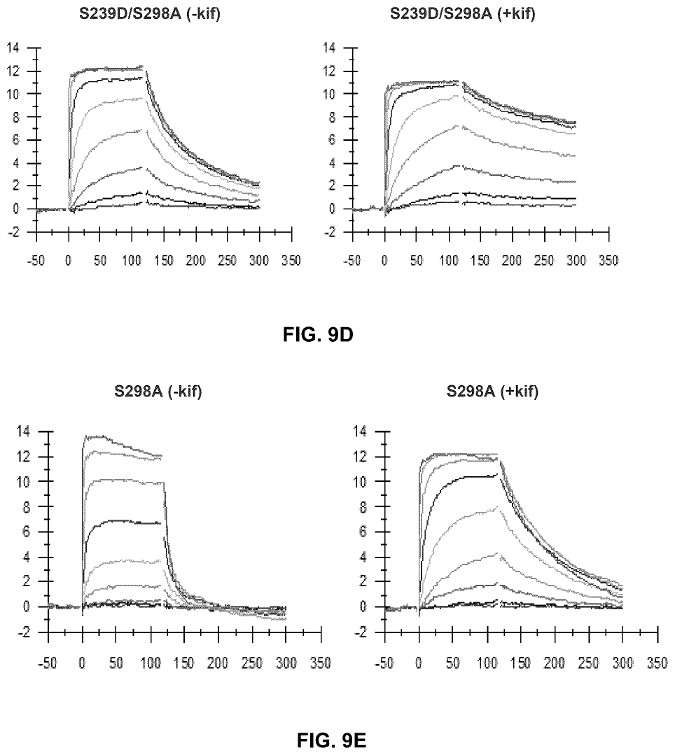

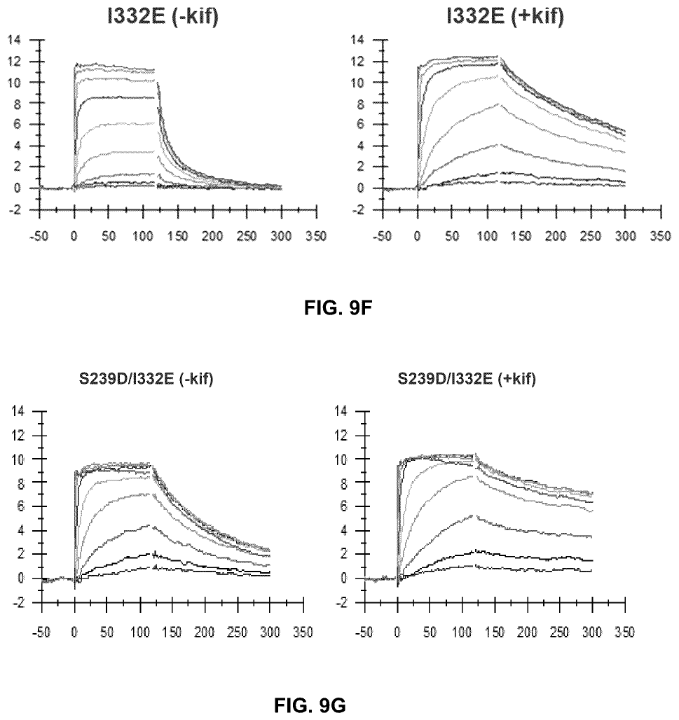

Human FcγRIIIa Binding Affinity Results: The binding affinity for human FcγRIIIa for various Fc variants with and without kifunensine treatment was measured and the sensorgams presented in FIG. 9B-G. FIG. 9A show the measured binding affinity metrics for the following antibodies: WT, S239D, S239D/S298A, S298A, I332E, and S239D/I332E. These results show that kifunensine treatment increases affinity to hFcγRIIIa 2.4- to 7-fold for all Fc variants tested and shows synergistic effects. For example, without kifunensine treatment, S239D/S298A has a lower affinity than S239D/I332E, but with kifunensine treatment, it shows a higher affinity than S239D/I332E without kifunensine treatment.

### Example 3. Rescuing the Loss of Thermal Stability Caused by Fc Mutation and Kifunensine Treatment

### Introduction

In this example, the thermal stability and Fcγ receptor binding of mAbs with Fc variants was tested, with and without kifunensine treatment. Since kifunensine treatment reduced the thermal stability of the antibodies, the ability of the R292C/V302C (disulfide) mutation to rescue the loss of thermal stability was tested.

### Materials and Methods

NanoDSF Thermal denaturation: mAbs (specific for protein 2 or protein 4) with Fc variants were diluted with buffer A (10 mM Histidine pH 6.0 at 1 mg/mL) and subjected to further buffer exchange into buffer A (4×), to remove traces of any salts from the purification process. The final concentration of all mAbs was then normalized to 0.5 mg/mL. Thermostability was determined by nano-format of Differential Scanning Fluorimetry (nanoDSF) with a Prometheus NT48 using “high sensitivity” capillaries and a linear gradient of temperature was applied from 20° to 95° C. at a heating rate of 1° C. per minute. NanoDSF uses the change in intrinsic fluorescence of proteins to monitor protein unfolding with increasing temperature. The protein solution was excited using a 266 nm wavelength light source and the fluorescence emission of the tyrosine and tryptophan residues at 330 nm and 350 nm was detected. The emission maxima and the intensity of tyrosine and tryptophan residues are highly dependent on their immediate environment and can change as the protein unfolds during thermal denaturation. Monitoring the change in ratio of fluorescence intensities at 330 nm and 350 nm as a function of temperature yields a sigmoid shaped curve that represents the unfolding transition of the protein. The midpoint of the sigmoid curve represents the melting temperature (Tm). The detectable temperature at which a protein begins to unfold is the Tonset. There are three inflexion points (IP) two correspond to the CH2 and CH3 of the Fc domain and the third one corresponds to Fab domain. Tagg is the temperature at which proteins exhibit a tendency to aggregate.

Fcγ Receptor Binding Analysis: Analysis of antibody binding to recombinant human FcγRIIIa-V158 using surface plasmon resonance (SPR) was performed on a Carterra LSA instrument using protein A capture. Antibodies specific for protein 4 were diluted to 0.5, 0.2, and 0.05 μg/mL in HBS-EP+ (10 mM HEPES pH 7.4, 150 mM NaCl, 3 mM EDTA, 0.05% surfactant P20) in duplicate wells of three 96-well plates. Each plate was printed to a sensor chip immobilized with protein A/G (Carterra PAGHC30M chip) in the capture array format in separate quadrants for 10 min. His-tagged recombinant human FcγRIIIa-V158 was serially diluted 2-fold in HBS-EP+pH 7.4 and injected over the capture antibodies for 2 min association followed by 3 min dissociation to measure the affinity. Each injection series contained a total of 12 concentrations of FcγRIIIa from 1.95 nM to 4000 nM. Buffer injections were evenly distributed between the receptor injections for proper blank subtraction. Sensorgrams were processed with the Carterra Kinetics analysis software and fit to a 1:1 binding model to obtain kinetic constants. Reported binding affinities were averaged from duplicates with plates and each antibody capture level.

### Results and Conclusions

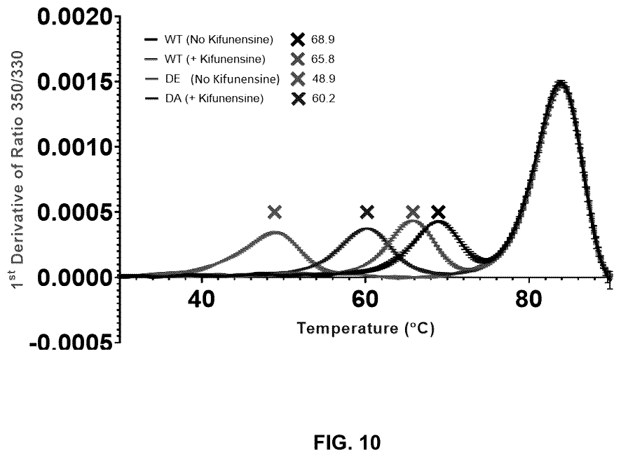

The Tm of the CH2 domains as determined by thermal denaturation for the antibodies specific for protein 2 with different Fc variants in the presence or absence of kifunensine treatment is shown in Table 5, below. Kifunensine treatment reduces the Tm of CH2 domain by 3 to 6° C. For the combination of the S239D/I332E Fc variant and kifunensine treatment the Tm is 42.8° C. versus 68.9° C. for the wildtype (WT) antibody without kifunensine treatment. Select antibodies of Table 5 (WT without kifunensine, WT with kifunensine, S239D/I332E without kifunensine, and S239D/S298A with kifunensine are presented in FIG. 10.

TABLE 5
Thermal denaturation by NanoDSF:
Antibodies Specific for Protein 2

| Antibody | | |
|---|---|---|
| IgG1 Fc Variant | +/−Kif | Tm (° C.) |
| WT | −Kif | 68.9 |
| | +Kif | 65.8 |
| +S239D | −Kif | 63.5 |
| | +Kif | 60.8 |
| +I332E | −Kif | 57.6 |
| | +Kif | 52.5 |
| +S298A | −Kif | 68.2 |
| | +Kif | 65 |
| +S239D/S298A | −Kif | 63 |
| | +Kif | 60.2 |
| +S239D/I332E | −Kif | 48.9 |
| | +Kif | 42.8 |

The Tm of the CH2 domains as determined by thermal denaturation for the antibodies specific for protein 4 with different Fc variants in the presence or absence of kifunensine treatment is shown in Table 6, shown below. Disulfide bond (DSB) between R292C/V302C, significantly rescues the loss of thermal stability caused by Fc variants and kifunensine treatment.

TABLE 6
Thermal denaturation by NanoDSF - Antibodies Specific for Protein 4

| IgG1 | | IgG1 | +R292C/ | +T256D/ | +T256D/T307Q + | |
|---|---|---|---|---|---|---|
| Fc | | WT | V302C | T307Q | R292C/V302C | +LS |
| Variant | +/−Kif | Tm (° C.) | Tm (° C.) | Tm (° C.) | Tm (° C.) | Tm (° C.) |
| IgG1 WT | −Kif | 71.4 | 73.1 | 65.3 | 73 | 66.7 |
| | +Kif | 65.5 | 73.1 | 61.1 | 73 | 64.7 |
| +S239D | −Kif | 64.1 | 73 | 59.5 | 72.3 | |
| | +Kif | 60.9 | 72.9 | 55.9 | 72 | |
| +S239D/ | −Kif | 63.2 | 72.9 | 59.2 | 72.1 | |
| S298A | +Kif | 60.3 | 72.7 | 55.3 | 72.2 | |
| +S239D/ | −Kif | 49.3 | 64.9 | 44.2 | 62.3 | |
| I332E | +Kif | 43 | 62.5 | 37.9 | 58.9 | |

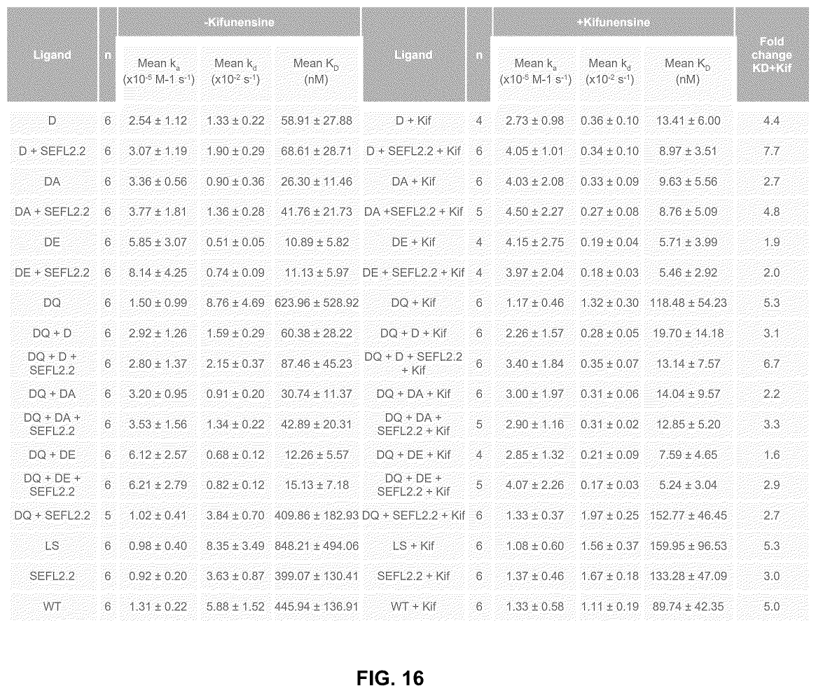

SPR results for binding to rhFcγRIIIa-V158 show WT-like binding affinities for R295C/V305C, T256D/T307Q, and T256D/T307Q+R295C/V305C. Kifunensine treatment increases affinity 1.6- to 7.7-fold for all Fc variants, as shown in Table 7 below and FIG. 16.

TABLE 7
Binding Affinity to rhFcγRIIIa-V158 of Antibodies Specific for Protein 4

| IgG1 | | | +R292C/ | +T256D/ | +T256D/T307Q + | |
|---|---|---|---|---|---|---|
| Fc | | IgG1 WT | V302C | T307Q | R292C/V302C | +LS |
| Variant | +/−Kif | KD (nM) | KD (nM) | KD (nM) | KD (nM) | KD (nM) |
| IgG1 WT | −Kif | 445.94 ± | 399.07 ± | 623.96 ± | 409.86 ± | 848.21 ± |
| | | 136.91 | 130.41 | 528.92 | 182.93 | 494.06 |
| | +Kif | 89.74 ± | 133.28 ± | 118.48 ± | 152.77 ± | 159.95 ± |
| | | 42.35 | 47.09 | 54.23 | 46.45 | 96.53 |
| +S239D | −Kif | 58.91 ± | 68.61 ± | 60.38 ± | 87.46 ± | |
| | | 27.88 | 28.71 | 28.22 | 45.23 | |
| | +Kif | 13.41 ± | 8.97 ± | 19.70 ± | 13.14 ± | |
| | | 6.00 | 3.51 | 14.18 | 7.57 | |
| +S239D/ | −Kif | 26.30 ± | 41.76 ± | 30.74 ± | 42.89 ± | |
| S298A | | 11.46 | 21.73 | 11.37 | 20.31 | |
| | +Kif | 9.63 ± | 8.76 ± | 14.04 ± | 12.85 ± | |
| | | 5.56 | 5.09 | 9.57 | 5.20 | |
| +S239D/ | −Kif | 10.89 ± | 11.13 ± | 12.26 ± | 15.13 ± | |
| I332E | | 5.82 | 5.97 | 5.57 | 7.18 | |
| | +Kif | 5.71 ± | 5.46 ± | 7.59 ± | 5.24 ± | |
| | | 3.99 | 2.92 | 4.65 | 3.04 | |

### Example 4. Effects of Kifunensine Treatment on the Characterization of the FcRn-Enhancing Variants

### Introduction

Experiments were done to characterize a series of variants of antibodies specific for protein 3 expressed in the presence and absence of kifunensine. As previous experiments had shown that kifunensine modifies the antibody's glycan structure to have high oligo-mannose and enhanced Fcγ receptor functionality, experiments were done to determine the effect on FcRn binding.

### Materials and Methods

N-linked glycan analysis: Glycan analysis was completed using mass spectrometry (as described above in Example 1).

Fcγ Receptor binding Analysis: FcγRIIIa binding was determined using SPR and measured on a Biacore™ T200 instrument. Recombinant human HPC4-tagged FcγRIIIa-V158 was diluted to 1.25 μg/mL in HBS-P+w/CaCl₂) (10 mM HEPES pH 7.4, 150 mM NaCl, 0.05% surfactant P20, 2 mM CaCl₂)) running buffer and injected to a CM5 chip was immobilized with anti-HPC4 tag antibody (Roche) for 30 sec at 5 μL/min flowrate. Antibodies were diluted to 500 nM in running buffer and injected in triplicate for 3 min over the captured receptor. Dissociation was measured for 3 min and the chip was regenerated with HBS-EP+buffer with 7 mM EDTA for 3 min at 20 μL/min flow rate. The steady state RU of 300 nM antibody was determined in triplicate and averaged. The fold change in response change relative to WT (reponse Fold Change) was determined for comparison between the variants in each backbone.

Human FcRn Binding Analysis: FcRn binding was determined using SPR and measured on a Biacore™ T200 instrument using the biotin CAPture kit (Cytiva). The running buffer was PBS with 0.05% surfactant P-20 (PBSP+, GE Healthcare) buffered with HCl for kinetics at pH 6.0 or binding at pH 7.4. CAPture reagent was captured on the CAP chip to a surface density of >2000 RU, followed by 0.2 μg/mL biotinylated recombinant human FcRn for 24 sec at 30 μL/min to a final surface density of ˜20 RU. Antibodies were serially diluted 3-fold from 1000 nM for a total of 5 concentrations in pH 6.0 running buffer and injected in duplicate for 3 min followed by 5 min dissociation in buffer. The surface was regenerated with 6 M guanidine hydrochloride, 250 mM NaOH for 2 min at 50 μL/min. Steady state RU measurements at pH 7.4 were obtained at 1000 nM in triplicate using the same conditions described above except for the capture level of FcRn was increased 10-fold. Kinetic parameters at pH 6.0 were fit to a bivalent model using the Biacore T200 Evaluation Software. Each concentration series was fit independently to obtain average rates and affinities. The apparent binding affinity was calculated for the first on and off rates from the bivalent model. Residual binding at pH 7.4 was measured using 1000 nM of each antibody at the same time for response comparison.

NanoDSF Thermal denaturation: Thermal stability was determined using DSF (as described above in Example 3) in triplicate of 0.2 mg/mL.

FcRn affinity Chromatograpy: pH dependence was determined using FcRn affinity chromatography with immobilized human FcRn and a MES-BTP pH gradient. The FcRn affinity column was adapted from Schlothauer et al. with biotinylated recombinant human FcRn on a 1 mL Streptavidin HP HiTrap column (GE Healthcare). The column was injected with 300 μg antibody in low pH buffer (20 mM 2-(N-morpholino) ethanesulfonic acid (MES, Sigma) pH 5.5; 150 mM NaCl) on an AKTA Pure System (AKTA). The antibodies were eluted by a pH gradient created with low and high pH buffer (20 mM 1,3-bis(tris(hydroxymethyl)methylamino) propane (Bis Tris Propane, Sigma) pH 9.5; 150 mM NaCl) over 30 column volumes (CV) at 0.5 mL/min and monitoring the absorbance and pH. The creation of a linear pH gradient (linear regression, R2>0.99) was achieved through the following stepwise format: 0-30% high pH buffer over 9 CV, 30-70% over 16.5 CV and 70-100% over 4.5 CV. The column was re-equilibrated with low pH buffer for subsequent runs. All variants were performed in triplicate. The FcRn affinity column elution profiles were fit to a single Gaussian distribution in Sigmaplot 11 (Systat Software, Inc.) to determine the elution volume, full width at half maximum (FWHM) and pH from at the UV280 maximum.

### Results

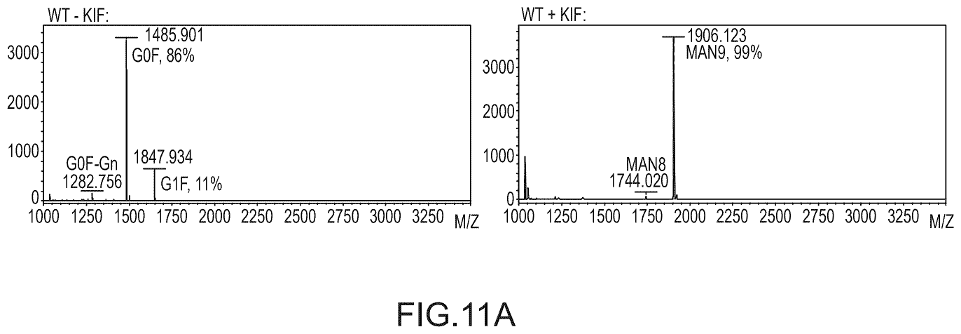

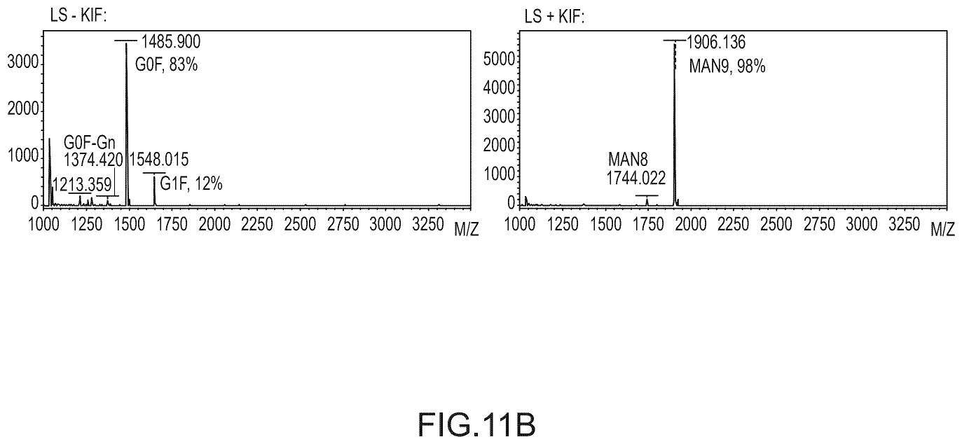

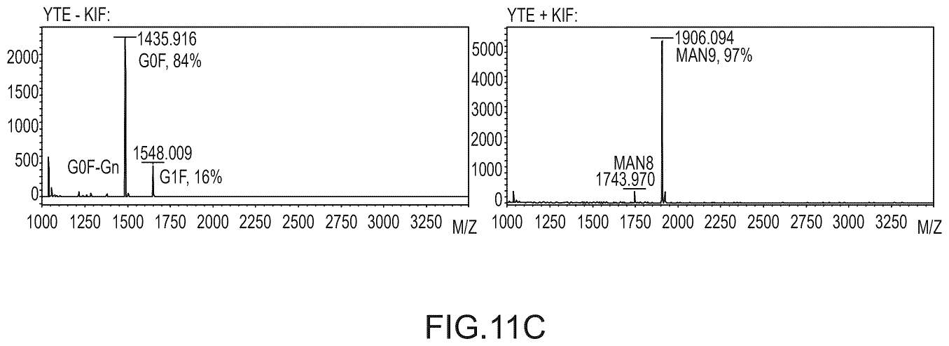

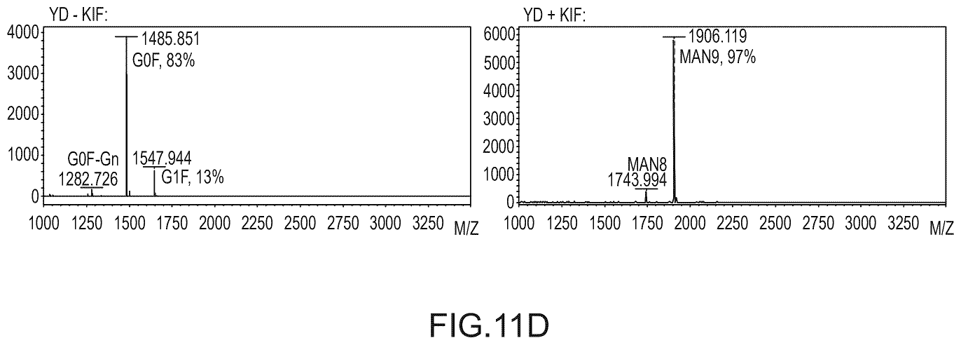

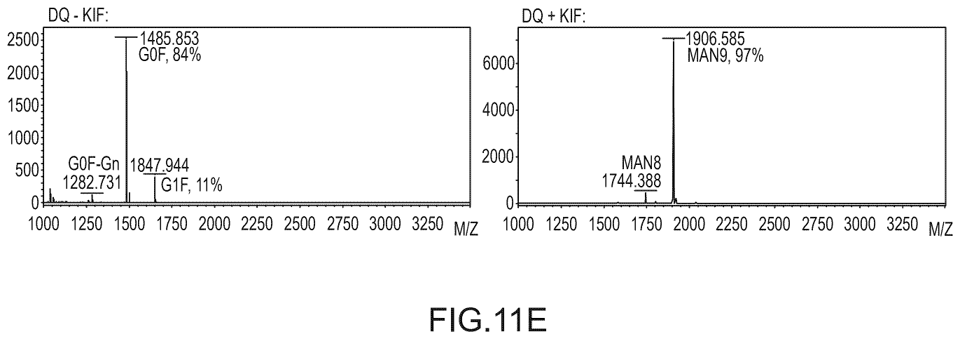

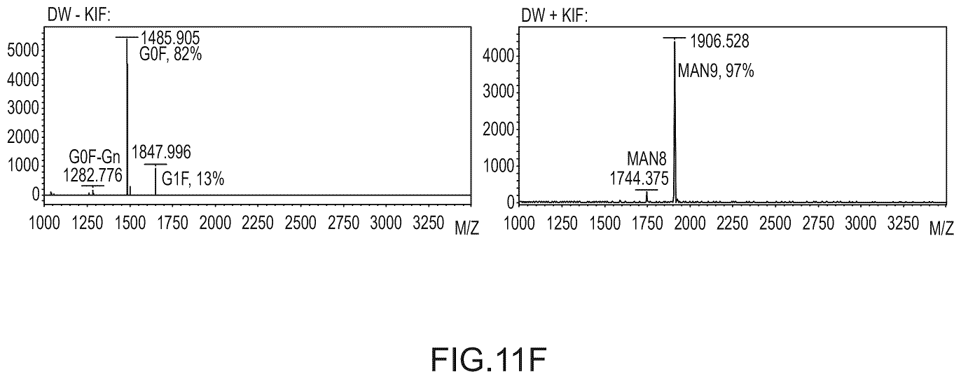

N-linked glycan analysis: FIG. 11A-F shows the mass spectrometry glycan analysis of WT, LS, YTE, YD, DQ, and DW antibodies specific for protein 3 with or without kifunensine treatment. All kifunesine treated antibodies have an oligomannose content of >97% Man₉(GlcNAc)₂. All untreated antibodies are >80% afucosylated.

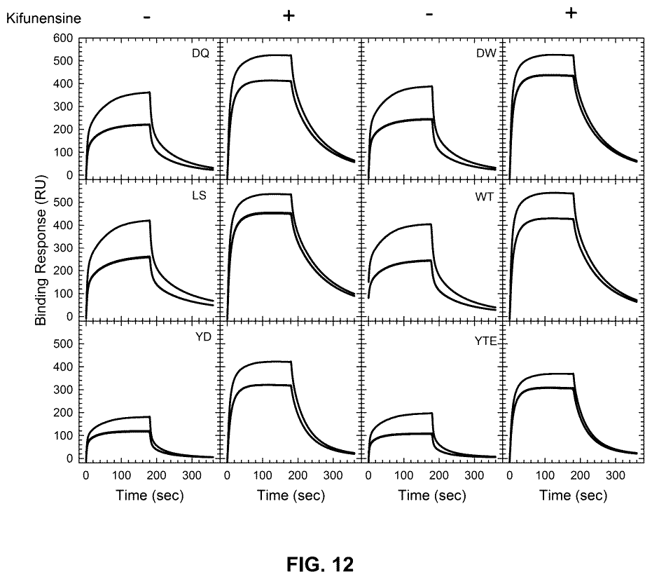

FcγRIIIa binding: FIG. 12 shows FcγRIIIa binding affinity response in triplicate for WT, DQ, DW, LS, YD, and YTE antibodies specific for protein 3 with and without kifunensine treatment. Table 8 below shows the fold change in FcγRIIIa binding affinity for antibodies specific for protein 3 with and without kifunensine. Table 9 shows the fold change in FcγRIIIa binding affinity for antibodies specific for protein 3 versus wildtype with kifunensine treatment. Enhanced FcγRIIIa binding was observed for all variants expressed with kifunensine, as well as the WT antibody.

TABLE 8
Fold Change (Kifunensine Treated vs Untreated)
in FcγRIIIa Binding Affinity

| | Fold |
|---|---|
| Variant | Change |
| DQ | 1.88 |
| DW | 1.79 |
| LS | 1.75 |
| WT | 1.76 |
| YD | 2.73 |
| YTE | 2.88 |

TABLE 9
Fold Change versus WT in FcγRIIIa Binding
Affinity with Kifunensine Treatment

| | Fold |
|---|---|
| Variant | Change |
| DQ + K | 1.69 |
| DW + K | 1.79 |
| LS + K | 1.85 |
| WT + K | 1.76 |
| YD + K | 1.31 |
| YTE + K | 1.26 |

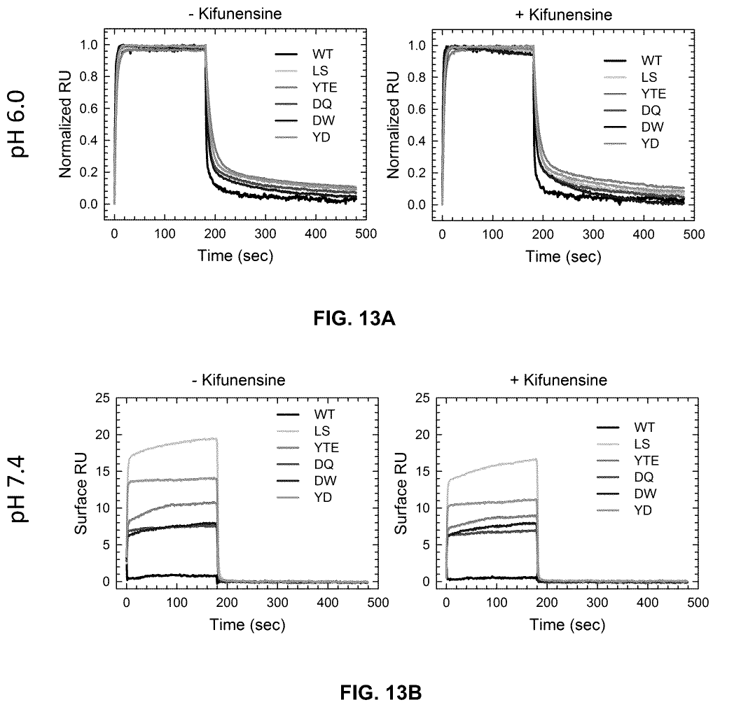

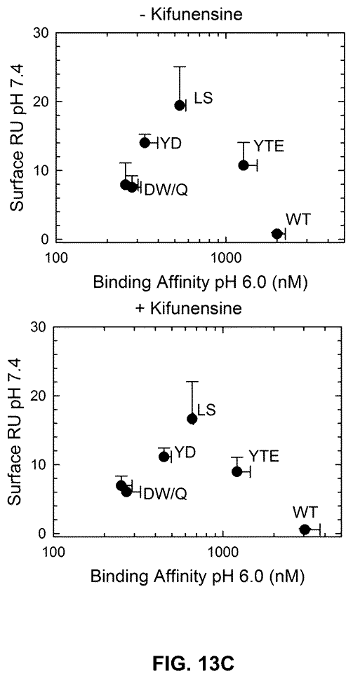

Human FcRn Binding: Human FcRn binding at pH 6.0 and pH 7.4 are shown in FIG. 13A and FIG. 13B, respectively. A scatter plot of the results at pH 6.0 is shown in FIG. 13C. FcRn binding assays were completed for WT, LS, YTE, DQ, DW, and YD antibodies specific for protein 3 with and without kifunensine treatment. At pH 6.0 no significant changes in the on or off binding rates were observed. DQ, DW and YD all have faster on and off binding rates compared to LS. At pH 7.0, slightly reduced human FcRn binding response was observed in the kifunesine treated samples. Table 10 below shows the binding data for the antibody variants in K_{D} or resonance units (RU) and SD at pH 6.0 and pH 7.4. The binding affinities and steady state RU for the treated and untreated antibodies are within the margin of error. No improvement in FcRn binding was observed.

TABLE 10
Comparison of FcRn binding at pH 6.0 and pH 7.4

| | |
|---|---|
| pH 6.0 | pH 7.4 |

| Variant | KD (nM) | SD (nM) | RU | SD RU |
|---|---|---|---|---|
| DQ | 280 | 36 | 7.5 | 1.7 |
| DQ + K | 270 | 56 | 6 | 1 |
| DW | 256 | 48 | 7.9 | 3.2 |
| DW + K | 251 | 40 | 6.9 | 1.4 |
| LS | 534 | 47 | 19.4 | 5.6 |
| LS + K | 658 | 10 | 16.6 | 5.4 |
| WT | 2000 | 240 | 0.8 | 0.2 |
| WT + K | 3040 | 700 | 0.5 | 0.2 |
| YD | 333 | 63 | 14 | 1.2 |
| YD + K | 449 | 47 | 11.1 | 1.3 |
| YTE | 1270 | 260 | 10.7 | 3.3 |
| YTE + K | 1210 | 240 | 9 | 2.1 |

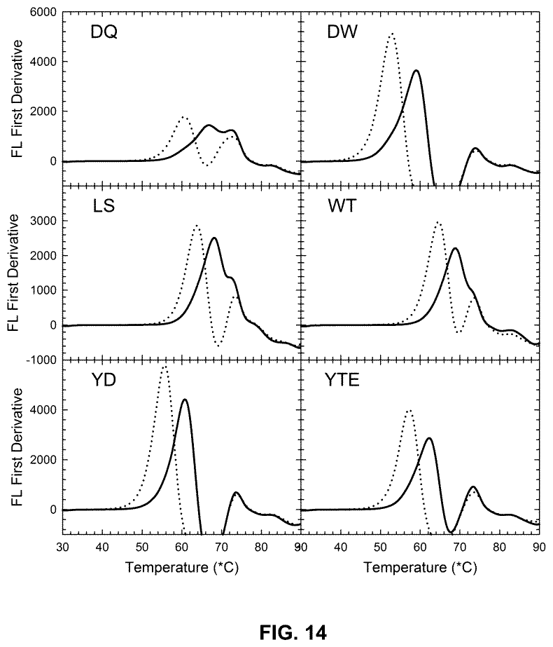

Thermal Stability: As shown in FIG. 14, thermal stability as determined by DSF for WT, LS, YTE, DQ, DW, and YD antibodies specific for protein 3 with and without kifunensine treatment was analyzed. The curves shown in solid black are without kifunensine treatment and the curves shown in the dotted line are with kifunensine treatment. Tm and change in Tm versus WT is shown in Table 11. Kifunensine treatment destabilizes every antibody variant a further 4-8° C. (versus the Fc mutation alone.) DW with kifunensine treatment shows a 16° C. decrease in thermal stability compared to the WT.

TABLE 11
Thermal Stability by DSF

| Variant | Tm | ΔTm vs WT |
|---|---|---|
| DQ | 67.0 | 2.0 |
| DQ + K | 60.5 | 8.5 |
| DW | 59.0 | 10.0 |
| DW + K | 53.0 | 16.0 |
| LS | 68.0 | 1.0 |
| LS + K | 64.0 | 5.0 |
| WT | 69.0 | — |
| WT + K | 64.5 | 4.5 |
| YD | 61.0 | 8.0 |
| YD + K | 55.5 | 13.5 |
| YTE | 62.0 | 7.0 |
| YTE + K | 57.0 | 12.0 |

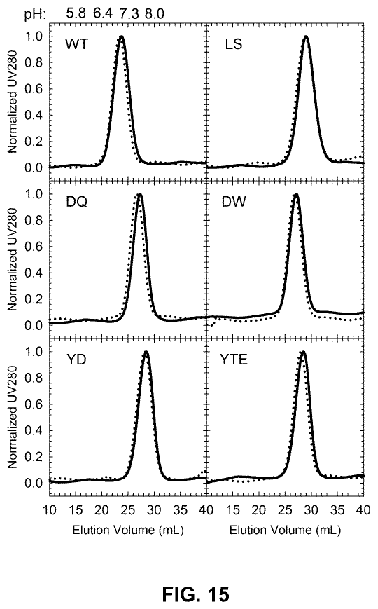

FcRn Affinity Chromatography: As shown in FIG. 15, FcRn affinity was determined by chromatography for WT, LS, YTE, DQ, DW, and YD antibodies specific for protein 3 with and without kifunensine treatment. The curves shown in solid black are without kifunensine treatment and the curves shown in the dotted line are with kifunensine treatment. The pH of elution for the antibody variants is provided in Table 12. The kifunensine treated samples show a similar pH elution profile as the untreated samples. This data supports the FcRn binding results showing little effect on overall binding affinity upon treatment with kifunensine.

TABLE 12
FcRn Affinity Chromatography, pH of elution

| Variant | pH |
|---|---|
| DQ | 7.58 |
| DQ + K | 7.50 |
| DW | 7.56 |
| DW + K | 7.51 |
| LS | 7.84 |
| LS + K | 7.81 |
| WT | 7.07 |
| WT + K | 7.01 |
| YD | 7.74 |
| YD + K | 7.70 |
| YTE | 7.74 |
| YTE + K | 7.68 |

### Conclusions

With regard to the analysis of N-glycan structures, it was determined that kifunensine treatment resulted in antibodies specific for protein 3 that have an oligomannose content of >97% Man₉(GlcNAc)₂. A significant improvement in FcγRIIIa binding was also observed, about 1.8 versus WT. There was little effect on FcRn binding affinity at pH 6.0. There was a slightly reduced binding response for FcRn binding at pH 7.4. Thermal stability of the antibodies was reduced by 4-8° C. with some Fc mutants being reduced by >12° C. versus WT. Finally, no effect on pH elution profile was observed.

### Example 5. FcγRIIIa Affinity of Stability Variants of Antibodies Specific for Protein 4 with and without Kifunensine

### Introduction

Experiments were done to compare human FcγRIIIa binding affinity of human IgG1 variants of antibodies specific for protein 4 with stability enhancing mutations and kifunensine treatment using SPR. The variants tested were S239D (D), S239D/S298A (DA), S239D/I332E (DE), R292C/V302C, T256D/T307Q (DQ) and combinations. M428L/N434S (LS) was included for comparison.

### Materials and Methods

Fcγ Receptor Binding Analysis: Analysis of antibody binding to recombinant human FcγRIIIa-V158 using surface plasmon resonance (SPR) was performed on a Carterra LSA instrument as described above in Example 3.

### Results and Conclusions

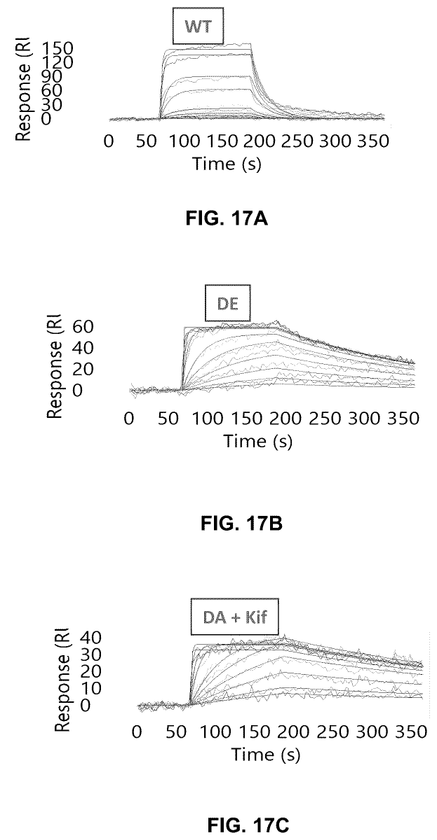

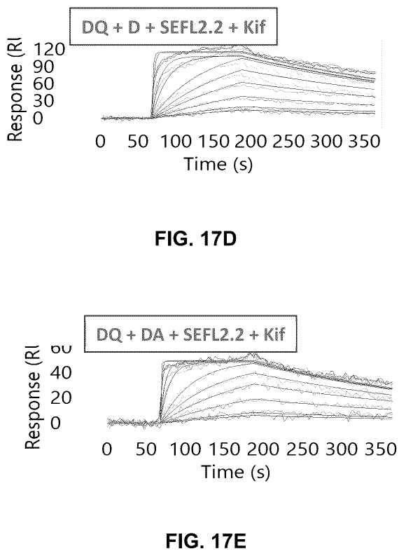

As shown in FIG. 16, the human FcγRIIIa binding affinity of various human IgG1 antibodies specific for protein 4 was tested with and without kifunensine treatment. The antibodies tested were as follows: WT, S239D (D), D+R292C/V302C (SEFL2.2), S239D/S298A (DA), DA+SEFL2.2, S239D/I332E (DE), DE+SEFL2.2, T256D/T307Q (DQ), DQ+D, DQ+D+SEFL2.2, DQ+DA, DQ+DA+SEFL2.2, DQ+DE, DQ+DE+SEFL2.2, DQ+SEFL2.2, LS, and SEFL2.2. FIG. 16 displays select binding affinity results including for the following antibodies: WT, D, DA, and DE. FIG. 17A-E depict select sensorgrams, those for WT (FIG. 17A), DE (FIG. 17B), DA with kifunensine treatment (FIG. 17C), DQ+D+R292C/V302C with kifunensine treatment (FIG. 17D), and DQ+DA+R292C/V302C with kifunensine treatment (FIG. 17E). These results show kifunensine treatment increases affinity to hFcγRIIIa 1.6- to 7.7-fold for all Fc variants tested. DE has the highest affinity with kifunensine treatment. R292C/V302C, DQ, DQ+R292C/V302C, and LS retained binding affinity similar to the WT antibody.

### Example 6. Human FcγRIIIa Affinity of Variants of Antibodies Specific for Protein 5

### Introduction

In order to test for enhanced effector function, human FcγRIIIa binding affinity of Fc stability mutants of antibodies specific for protein 5 treated with or without kifunensine was measured.

### Materials and Methods

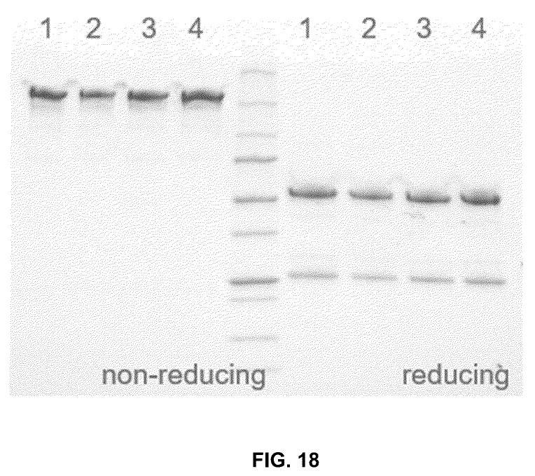

The afucosylated antibody specific for protein 5 was obtained from Creative Biolabs. To measure Fcγ receptor IIIa binding, antibodies were diluted to 1 μg/mL and captured to a protein A chip for 30 sec at 10 μL/min with HBS-EP+pH 7.4 running buffer (Biacore™ T200 instrument). Recombinant human FcγRIIIa-V158 was serially diluted 3-fold from 111.111 nM to 1.372 nM in HBS-EP+. The 5 concentrations of rhFcγRIIIa-were injected over the captured antibodies for 2 min, followed by 3 min of dissociation at 30 μL/min flow. The surface was regeneration with 10 mM glycine-HCl pH 1.5 for 30 sec at 20 μL/min with 1 min stabilization. The sensorgrams were processed and fit to a 1:1 kinetic binding model. SDS-PAGE was done with reducing and non-reducing conditions as described in Example 1 (FIG. 18.) Glycan analysis was done using MALDI-TOF mass spectra as described in Example 1. Binding affinity to protein 5 was determined using Octet (Forte Bio) with HIS2 biosensors. Thermal stability was determined by NanoDSF as described in Example 3 above.

### Results and Conclusions

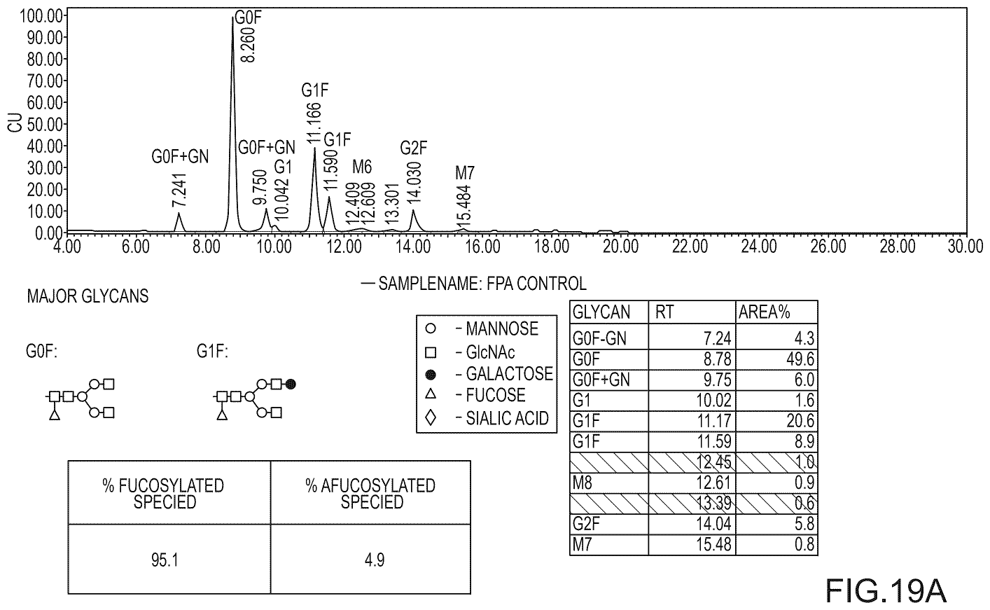

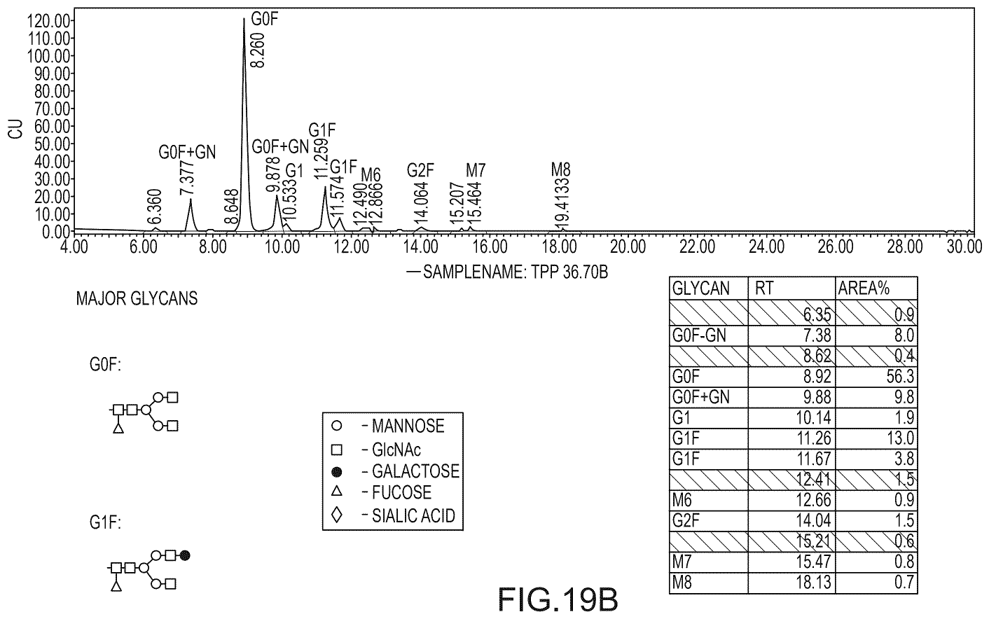

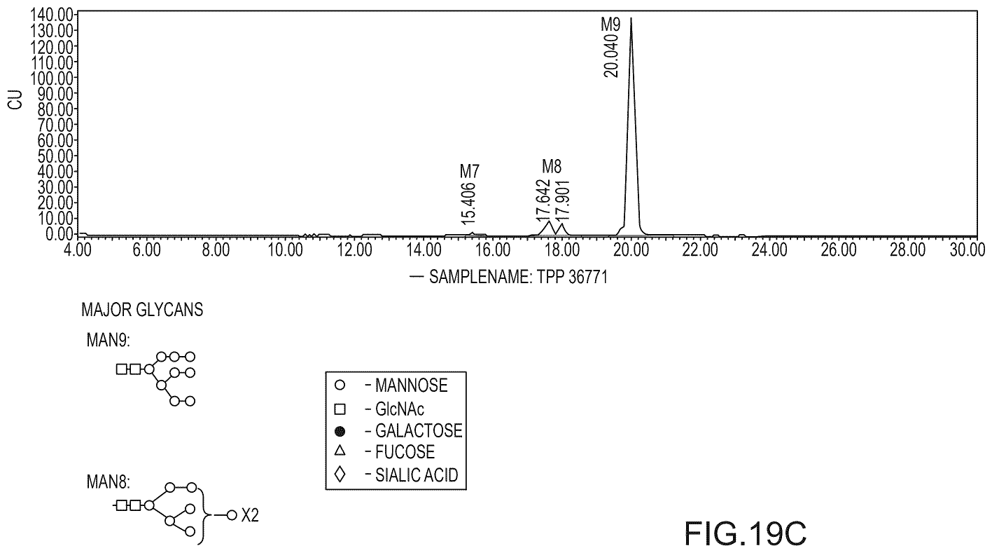

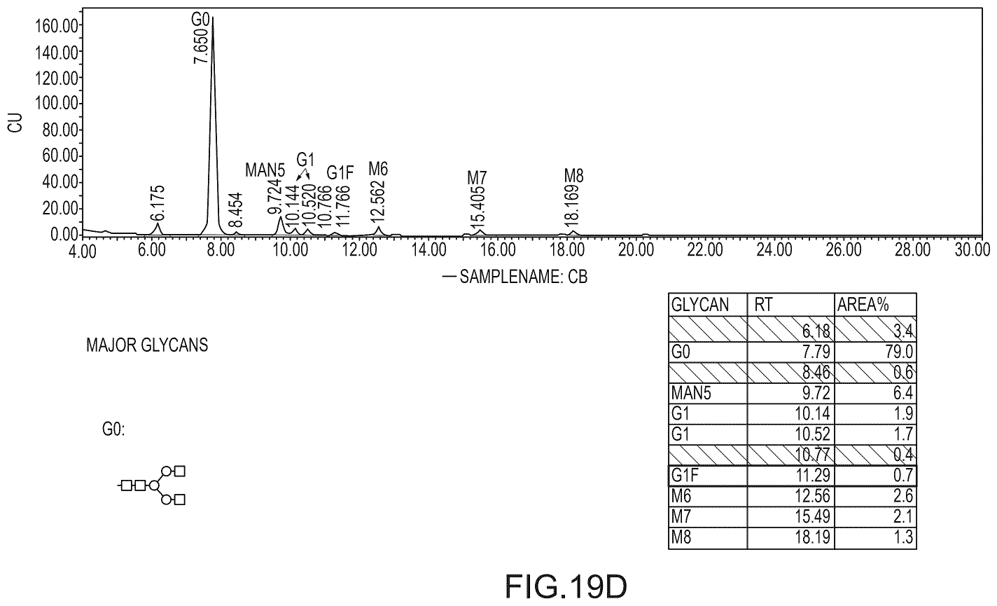

Glycan Analysis: As shown in FIG. 19A-D, MALDI-TOF glycan analysis of the antibodies specific for protein 5 was completed. For the WT antibody, the major glycans were determined to be G0F and G1F (FIG. 19A.) The WT antibodies were also determined to be 95.1% fucosylated and 4.9% afucosylated. For the DE+R292C/V302C antibody, the major glycans were determined to be G0F and G1F (FIG. 19B.) For the DA+kifunensine antibody, the major glycans were determined to be Man₉(GlcNAc)₂ and Man8 (GlcNAc)₂ (FIG. 19C.) For the afucosylated antibody, the major glycan was determined to be G0 (FIG. 19D.)

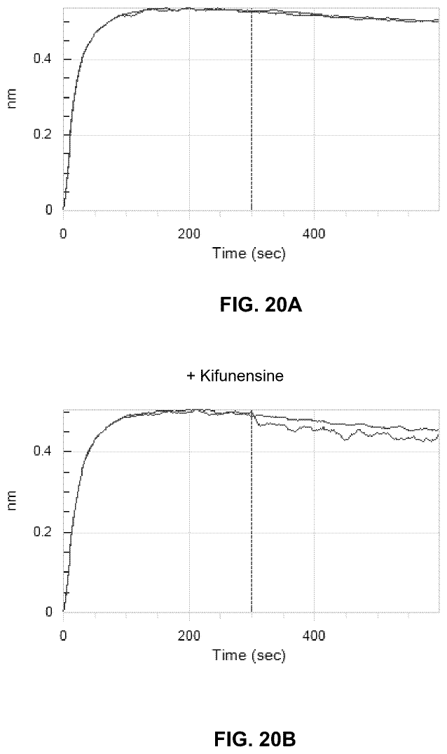

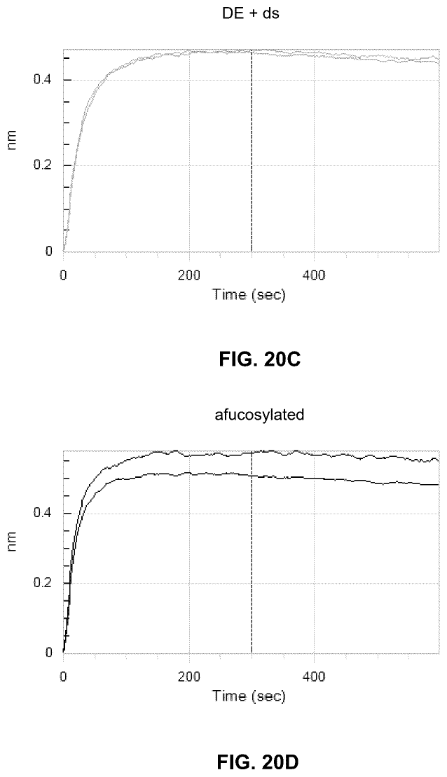

Binding Affinity to Protein 5: FIG. 20A-D depicts sensorgrams of binding analysis of various antibodies to protein 5 including WT (FIG. 20A), DA+kifunensine (FIG. 20B), DE+R292C/V302C (disulfide) (FIG. 20C), and afucosylated (FIG. 20D.)

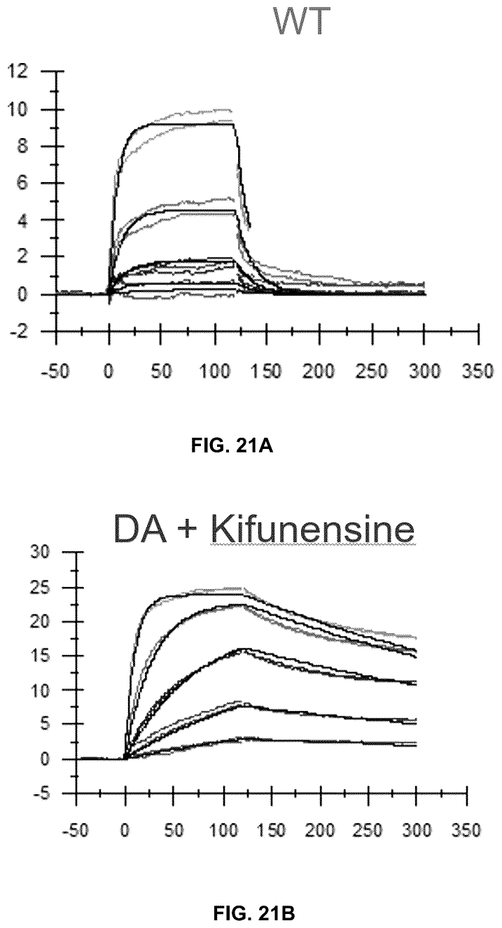

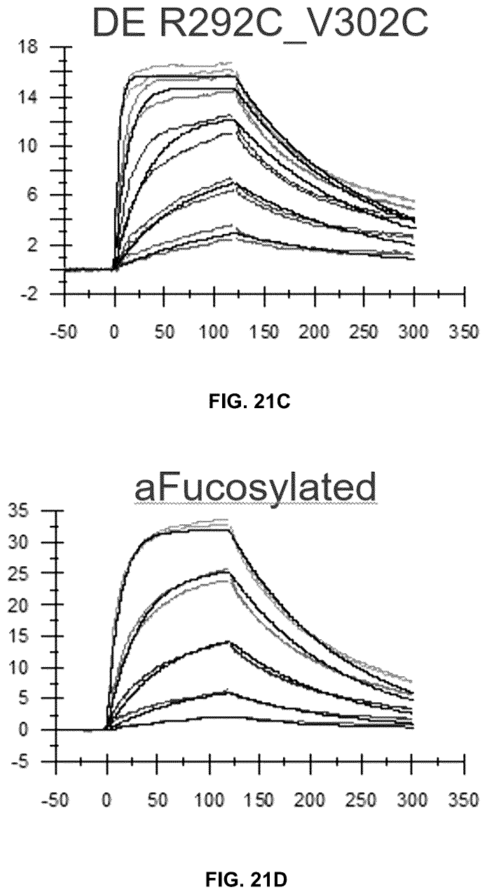

Human FcγRIIIa Affinity of Antibodies Specific for Protein 5: As shown in FIG. 21A-D the binding affinity to human FcγRIIIa of various antibodies specific for protein 5 was tested including WT (FIG. 21A), DA+kifunensine (FIG. 21B), DE+R292C/V302C (disulfide) (FIG. 21C), and afucosylated (FIG. 21D). The binding affinity metrics for all variants are provided in Table 13, below. All variants showed a higher binding affinity for human FcγRIIIa over WT.

TABLE 13
Binding Affinity to Human FcγRIIIa of Antibodies Specific to Protein 5

| | | | | | | Fold |
|---|---|---|---|---|---|---|
| Antibody | Info | kₐ (1/Ms) | k_{d} (1/s) | KD (M) | % Rₘₐₓ | KD WT |
| WT | TPP-23424 | 8.95E+05 | 1.06E−01 | 1.18E−07 | 85.1 | 1 |
| DA + Kifunensine | TPP-36771 | 9.00E+05 | 2.32E−03 | 2.57E−09 | 127.6 | 46 |
| DE (R292C/V302C) | TPP-35708 | 2.20E+06 | 8.32E−03 | 3.79E−09 | 133.9 | 31 |
| aFucosylated | Creative | 6.22E+05 | 9.25E−03 | 1.49E−08 | 124.9 | 8 |
| | Biolabs | | | | | |

Thermal Stability: The thermal stability of the various antibodies specific for protein 5 as determined by NanoDSF is provided in Table 14, below. Fc mutations DA and DE lower the thermal stability of the CH2 domain but not the Fab domain.

TABLE 14
Thermal Stability of Antibodies Specific for Protein 5

| | T_{M}1 (° C.), | T_{M}2 (° C.), |
|---|---|---|
| Sample Name | CH2 domain | Fab |
| WT IgG1 | 70.5 ± 0.0 | 77.6 ± 0.1 |
| DA + Kifunensine | 61.0 ± 0.4 | 77.6 ± 0.3 |
| DE (R292C/V302C) | 67.5 ± 0.2 | 77.9 ± 0.1 |
| aFucosylated | 70.7 ± 0.2 | 77.8 ± 0.1 |

### Example 7. Engineering Thermally Stable Fc Domains with Modulated Fc Function for Effective Biotherapeutics

### Introduction

285 single point Fc mutations were generated using saturation mutagenesis on 1) the CH2 loop residues without perturbing the hydrophobic core residues, like I332, and 2) CH2-CH3 interface residues. The saturation library at these residues was screened for thermal stability and hFcγRIIIa binding. Mutations were identified that had minimal impact on thermal stability and enhanced FcγR binding (in particular, hFcγRIIIa binding). The variants expressed were analyzed with and without the presence of kifunensine.

### Materials and Methods

Fc Fragments were captured to immobilized protein AG and flow with multiple concentrations of hFcγRIIIa-V158 was used to measure binding.

Sample preparation: PEPP samples were filtered at 0.22 M in a 96-well filter plate. A280 was measured on Stunner. Samples were normalized to 200 μg/mL in PBS at pH 7.2 on Hamilton. The sample was diluted to 20 μg/mL in 96 well plates, then to 2.5 μg/mL in 384 well plates with Benchsmart.

Procedure on Carterra LSA: Protein AG was immobilized to a HC30M sensor chip using amine chemistry. Load antibodies were diluted to 0.25 μg/mL or 5 μg/mL per 10 minutes in multiple prints. Multiple concentrations of hFcγRIIIa-V158 were injected for 2 minutes with 5 minutes of dissociation, including twelve 2-fold serial dilutions from 4000 nM to 1.9 nM in HBS-EP+ at pH 7.4. The chips were regenerated with 3×30 seconds of 10 mM glycine at pH 1.5 with 1 minute stabilization. Finally, the sensorgrams were fit using a 1:1 kinetic binding model.

Thermal Stability: The thermal stability of the single point Fc mutations with and without kifunensine was measured by nanoDSF. Buffer exchange into 10 mM histidine pH 6.0 was completed using the top 46 mutants with higher affinity than WT (with and without kifunensine).

### Results and Conclusions

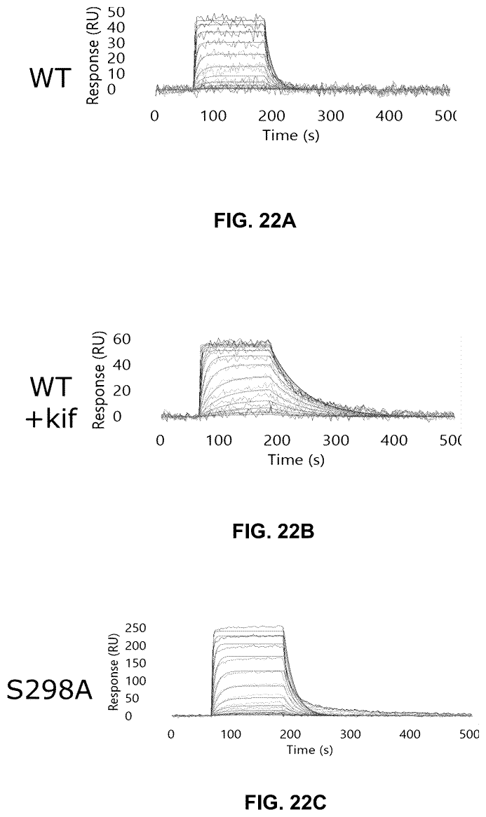

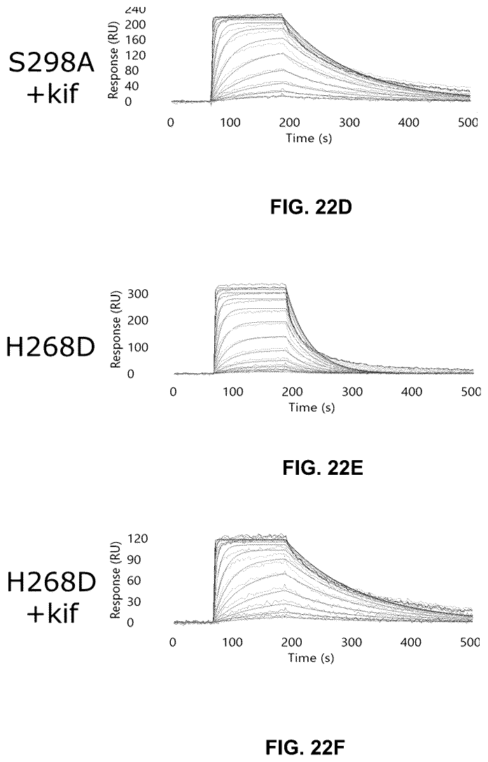

FIGS. 22A-F depict sensorgrams of human FcγRIIIa binding affinity for the following antibodies: WT (FIG. 22A), WT with kifunensine treatment (FIG. 22B), S298A (FIG. 22C), S298A with kifunensine treatment (FIG. 22D), H268D (FIG. 22E), and H268D with kifunesine treatment (FIG. 22F). Both variants showed a higher binding affinity for human FcγRIIIa over WT.

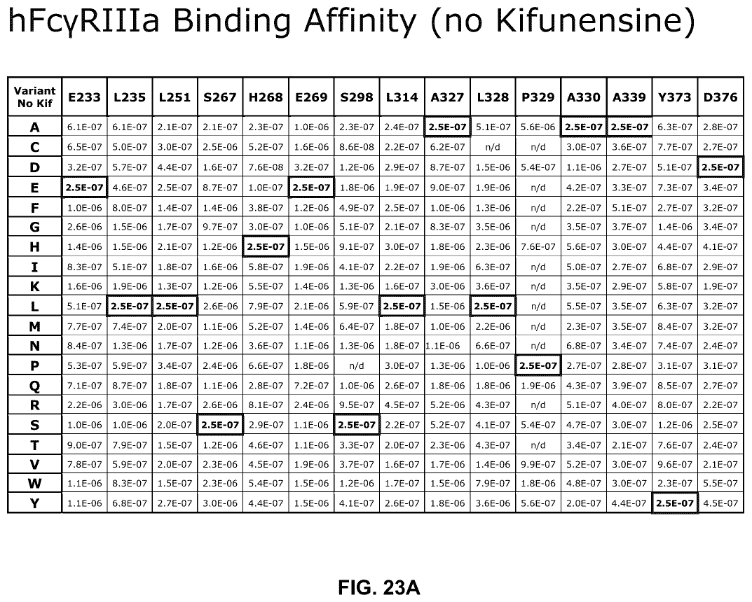

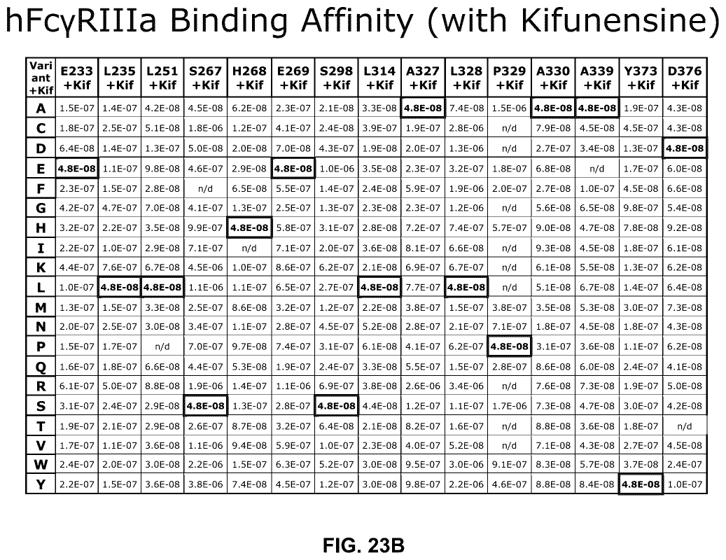

FIGS. 23A-B present the numerical values for human FcγRIIIa binding affinity without kifunesine (FIG. 23A) and with kifunesine (FIG. 23B). Values for WT in FIG. 23A and FIG. 23B are shown in bold. These figures show that for some variants tested, binding affinity for human FcγRIIIa is increased versus WT. For example, FIG. 23A shows that for WT not treated with kifunensine, KD (M) is 2.5E-07 and for S298C not treated with kifunensine, KD (M) is 8.6E-08. FIG. 23B shows the binding affinity is even further enhanced with kifunensine and the KD (M) value for S298C with kifunensine treatment is 2.4E-08.

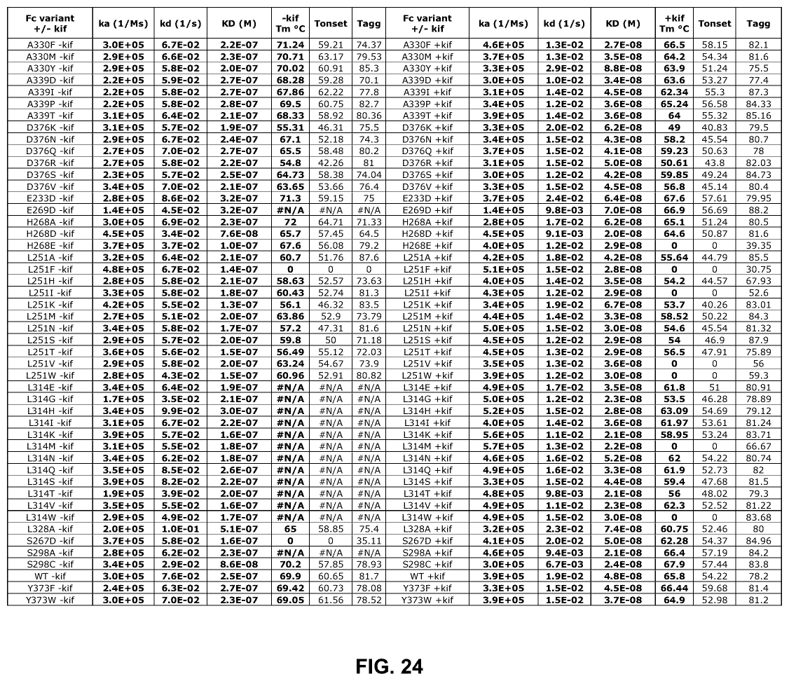

FIG. 24 presents the numerical values for human FcγRIIIa binding affinity with and without kifunesine, as well as the Tm values for the Fc variants tested and WT. This figure shows that for some variants tested, binding affinity for human FcγRIIIa is increased versus WT. For example, for A330A (WT) not treated with kifunensine, KD (M) is 2.5E-07 and for A330F not treated with kifunensive, KD (M) is 2.2E-07. Binding affinity is further enhanced with kifunensine and the KD (M) value for A330F with kifunensine treatment is 2.7E-08 and the Tm (about 66.5° C.) is within 5 degrees Celsius of the WT cultured in the absence of kifunensine (69.9° C.).

Table 15, below, shows the Fc variants selected for optimal hFcγRIIIa binding affinity and thermal stability. The Fc variants shown below (for which Tm data is available) had comparable or enhanced binding affinity versus WT, when both were cultured without kifunensine. When both were cultured with kifunensine, the Fc variants had higher binding affinity versus WT. Additionally, the Tm of these variants was within 5 degrees Celsius of WT. For example, Table 16 illustrates that the binding affinity of H268D is enhanced with kifunensine with a KD (M) of 2.0E-08 versus a KD (M) of 7.6E-08 with no kifunensine. Furthermore, for H268D the Tm is 64.6° C. with kifunensine which is about one degree Celsius less than the Tm without kifunensine, which is 65.7° C. Furthermore, the Tm of H268D with kifunensine is within 10 degrees Celsius of the Tm of a binding polypeptide with a WT Fc domain cultured in the absence of kifunensine, which is 69.9° C.

TABLE 15
Fc Variants Selected for Optimal hFcγRIIIa
Binding Affinity and Thermal Stability

| | No Kif | +Kif | No Kif | +Kif |
|---|---|---|---|---|
| Mutation | Tm ° C. | Tm ° C. | KD (M) | KD (M) |
| A330F | 71.2 | 66.5 | 2.2E−07 | 2.7E−08 |
| A330M | 70.7 | 64.2 | 2.3E−07 | 3.5E−08 |
| A339D | 68.3 | 63.6 | 2.7E−07 | 3.4E−08 |
| A339I | 67.9 | 62.3 | 2.7E−07 | 4.5E−08 |
| A339P | 69.5 | 65.2 | 2.8E−07 | 3.6E−08 |
| A339T | 68.3 | 64.0 | 2.1E−07 | 3.6E−08 |
| H268D | 65.7 | 64.6 | 7.6E−08 | 2.0E−08 |
| H268E | 67.6 | | 1.0E−07 | 2.9E−08 |
| L314I | 66.6 | 62.0 | 2.2E−07 | 3.6E−08 |
| L314M | 69.3 | | 1.8E−07 | 2.2E−08 |
| L314Q | 64.9 | 61.9 | 2.6E−07 | 3.3E−08 |
| L314W | | | 1.7E−07 | 3.0E−08 |
| S267D | | 62.3 | 1.6E−07 | 5.0E−08 |
| S298A | 69.0 | 66.4 | 2.3E−07 | 2.1E−08 |
| S298C | 70.2 | 67.9 | 8.6E−08 | 2.4E−08 |
| Y373F | 69.4 | 66.4 | 2.7E−07 | 4.5E−08 |
| Y373W | 69.1 | 64.9 | 2.3E−07 | 3.7E−08 |

1-44. (canceled)

145. A binding polypeptide comprising a Fc domain, wherein the Fc domain comprises an aspartic acid (D) at amino acid position 239 and a glutamic acid (E) at amino acid position 332, according to EU numbering, and
wherein the Fc domain is afucosylated.

146. The binding polypeptide of claim 145, wherein the Fc domain further comprises a cysteine (C) at amino acid position 292 or a C at amino acid position 302.

147. The binding polypeptide of claim 145, wherein the Fc domain further comprises a C at amino acid position 292 and a C at amino acid position 302.

148. The binding polypeptide of claim 145, comprising an increased antibody-dependent cellular cytotoxicity (ADCC) activity compared to a reference polypeptide.

149. The binding polypeptide of claim 148, wherein the reference polypeptide either comprises a wild-type Fc domain or a Fc domain comprising a D at amino acid position 239 and a E at amino acid position 332, according to EU numbering, wherein the Fc domain is not afucosylated.

150. The binding polypeptide of claim 148, wherein the ADCC activity of the binding polypeptide is at least 1, 1.5, 2, 2.5 or 3-fold higher in ADCC activity compared to a reference polypeptide.

151. The binding polypeptide of claim 145, wherein the binding polypeptide has an increased affinity for binding to an Fcγ receptor compared to a reference polypeptide.

152. The binding polypeptide of claim 151, wherein the Fcγ receptor is human FcγRIIIa (hFcγRIIIa).

153. The binding polypeptide of claim 151, wherein the reference polypeptide comprises either a wild-type Fc domain or a Fc domain comprising a D at amino acid position 239 and a E at amino acid position 332, according to EU numbering, wherein the Fc domain is not afucosylated.

154. The binding polypeptide of claim 153, wherein the binding polypeptide specifically binds to hFcγRIIIa with:
(a) a KD (nM) less than about 139-fold;
(b) a k_{d} of less than about 36-fold; or
(c) a kₐ of more than about 4-fold
difference when compared to a wild-type Fc domain.

155. The binding polypeptide of claim 145, wherein the Fc domain comprises an immunoglobulin G (IgG) Fc domain.

156. The binding polypeptide of claim 155, wherein the Fc domain comprises an IgG1 Fc domain.

157. A binding polypeptide comprising a Fc domain
wherein the Fc domain comprises a D at amino acid position 239 and an E at amino acid position 332, according to EU numbering,
wherein the Fc domain is afucosylated, and
wherein the binding polypeptide that specifically binds to FcγR with:
(a) a KD of about 4 nM;
(b) a k_{d} of about 0.3×10⁴ s⁻¹; or
(c) a kₐ of about 7.5×10⁵ M⁻¹s⁻¹; and
optionally wherein the Fc domain further comprises a C at amino acid position 292 or a C at amino acid position 302 according to EU numbering.

158. An isolated nucleic acid molecule comprising a nucleic acid encoding the binding polypeptide of claim 145.

159. A vector comprising the isolated nucleic acid molecule of claim 158.

160. A host cell comprising the vector of claim 159.

161. The binding polypeptide of claim 145, wherein the binding polypeptide is comprised in a pharmaceutical composition.

162. A method of treating a disease or disorder in a subject in need thereof, comprising administering to the subject an effective amount of the pharmaceutical composition comprising:
a binding polypeptide comprising a Fc domain, wherein the Fc domain comprises a D at amino acid position 239 and an E at amino acid position 332, according to EU numbering, and
wherein the Fc domain is afucosylated, and
optionally wherein the Fc domain further comprises a C at amino acid position 292 or a C at amino acid position 302, according to EU numbering.

163. A method of making the binding polypeptide of claim 145 comprising the steps of:
(a) expressing a nucleic acid comprising a nucleic acid encoding a Fc domain, wherein the Fc domain comprises a D at amino acid position 239 and a E at amino acid position 332, according to EU numbering in a cell line with altered glycosylation; and
(b) purifying the binding polypeptide;
optionally wherein the Fc domain further comprises a C at amino acid position 292 or a C at amino acid position 302 according to EU numbering.
